(19) United States
(12) Patent Application Publication
(10) Pub. No.: US 2009/0136954 A1
(43) Pub. Date: May 28, 2009

(54) GENETIC MARKERS FOR SCD OR SCA THERAPY SELECTION

(75) Inventors: Orhan SOYKAN, Shoreview, MN (US); Tara NAHEY, Minneapolis, MN (US); Jeffrey LANDE, Minneapolis, MN (US)

Correspondence Address: Hahn & Voight-Medtronic, Inc. patent applications, 1012 14th Street, NW Suite 620, Washington, DC, 20005, US

(73) Assignee: MEDTRONIC, INC., Minneapolis, MN (US)

(21) Appl. No.: 12/271,338

(22) Filed: Nov. 14, 2008

Related U.S. Application Data
(60) Provisional application No. 60/987,968, filed on Nov. 14, 2007.

(51) Int. Cl. C12Q 1/68; C07H 21/04

(52) U.S. Cl. 435/6; 536/23.1; 536/24.33

(57) ABSTRACT

Variations in certain genomic sequences useful as genetic markers of Sudden Cardiac Death (“SCD”), or Sudden Cardiac Arrest (“SCA”) risk, are described. Novel genetic markers useful in assessing the risk of SCD, or SCA, and kits containing the same are provided herein. Methods of distinguishing patients having an increased susceptibility to SCD, or SCA, through use of these markers, alone or in combination with other markers, are also provided. Further, methods of assessing the need for an Implantable Cardio Defibrillators (“ICD”) in a patient are taught.

### CROSS-REFERENCE TO RELATED APPLICATIONS

This application claims priority to U.S. Provisional Application Ser. No. 60/987,968, filed Nov. 14, 2007.

### REFERENCE TO SEQUENCE LISTING

This application contains a Sequence Listing submitted as an electronic text file named “Seq_List_ST25.txt”, having a size in bytes of 184 kb, and created on Nov. 13, 2008. Two compact discs are made part of the specification. The first compact disc is the “Sequence Listing”. The second disc is an exact duplicate of the first and is the Computer Readable File (“CRF”) required under Rule § 1.821(e). The information contained in the “Sequence Listing” is hereby incorporated by reference.

### BACKGROUND

Implantable Cardio Defibrillators (“ICD”) effectively terminate life threatening ventricular tachy-arrhythmias, such as ventricular tachycardias (“VT”) and ventricular fibrillation (“VF”). For many patients, ICDs are indicated for various cardiac related ailments including myocardial infarction, ischemic heart disease, coronary artery disease, and heart failure. The use of these devices, however, remains low due in part to lack of reliable markers to select patients who are in need of these devices. Hence, despite the effectiveness of ICDs in Sudden Cardiac Death (“SCD”) or Sudden Cardiac Arrest (“SCA”) prevention, many susceptible patients who might benefit from an ICD do not receive one due to a lack of reliable methods for the identification of SCD or SCA.

### SUMMARY OF THE INVENTION

Novel genetic markers useful in assessing the risk of Sudden Cardiac Death (“SCD”) and Sudden Cardiac Arrest (“SCA”) are provided herein. Methods of distinguishing patients having an increased susceptibility to SCD, or SCA, through use of these markers, alone or in combination with other markers, are also provided. Further, methods of assessing the need for an ICD in a patient are taught. Specifically, an isolated nucleic acid molecule is contemplated that is useful to predict SCD, or SCA risk, and Single Nucleotide Polymorphisms (“SNPs”) selected from the group of SEQ ID NO.'s 1-822 that can be used in the diagnosis, distinguishing, and detection thereof.

Also contemplated are isolated nucleotides useful to predict SCD, or SCA risk, complementary to any one of SEQ ID NO.'s 1-822 where the complement is between 3 to 101 nucleotides in length and overlaps a position 51 in any of the SEQ ID NO.'s 1-822, which represents a SNP. An amplified nucleotide is further contemplated containing a SNP embodied in any one of SEQ ID NO.'s 1-822, or a complement thereof, overlapping position 51, wherein the amplified nucleotide is between 3 and 101 base pairs in length. The lower limit of the number of nucleotides in the isolated nucleotides, and complements thereof, can range from about 3 base pairs from position 50 to 52 in any one of SEQ ID NO.'s 1-822 such that the SNP at position 51 is flanked on either the 5′ and 3′ side by a single base pair, to any number of base pairs flanking the 5′ and 3′ side of the SNP sufficient to adequately identify, or result in hybridization. This lower limit of nucleotides can be from about 3 to 99 base pairs, the optimal length being determinable by a person of ordinary skill in the art. For example, the isolated nucleotides or complements thereof, can be from about 5 to 101 nucleotides in length, or from about 7 to 101, or from about 9 to 101, or from about 15 to 101, or from about 20 to 101, or from about 25 to 101, or from about 30 to 101, or from about 40 to 101, or from about 50 to 101, or from about 60 to 101, or from about 70 to 101, or from about 80 to 101, or from about 90 to 101, or from about 99 to 101 nucleotides, so long as position 51 in any of SEQ ID NO.'s 1-822 is overlapped. Preferred primer lengths can be from 25 to 35, 18 to 30, and 17 to 24 nucleotides.

A method of distinguishing patients having an increased susceptibility to SCD or SCA from patients who do not is contemplated, by detecting at least one SNP at position 51 in any of SEQ ID NO.'s 1-822 in a nucleic acid sample from the patients wherein the presence or absence of the SNP can be used to assess increased susceptibility to SCD or SCA.

A method of determining SCA or SCD risk in a patient is also contemplated which requires identifying one or more SNP at position 51 in any of SEQ ID NO.'s 1-822 in a nucleic acid sample from the patient.

A method for determining whether a patient needs an Implantable Cardio Defibrillators (“ICD”) is contemplated by identifying one or more SNPs at position 51 in any of SEQ ID NO.'s 1-822 in a nucleic acid sample from the patient.

A method of detecting SCA or SCD-associated polymorphisms is further contemplated by extracting genetic material from a biological sample and screening the genetic material for at least one SNP in any of SEQ ID NO.'s 1-822, which is at position 51.

Those skilled in the art will recognize that the analysis of the nucleotides present in one or several of the SNP markers in an individual's nucleic acid can be done by any method or technique capable of determining nucleotides present at a polymorphic site. One of skill in the art would also know that the nucleotides present in SNP markers can be determined from either nucleic acid strand or from both strands.

### BRIEF DESCRIPTION OF THE DRAWINGS

The foregoing and other features and aspects of the present disclosure will be best understood with reference to the following detailed description of a specific embodiment of the disclosure, when read in conjunction with the accompanying drawings, wherein:

FIG. 1 depicts increase in the Number Needed to Treat (“NNT”) observed for the ICD therapy as devices are implanted in patients with lower risks.

FIG. 2 is a flow chart of a MAPP sub-study design. MAPP was a preliminary genetic association study conducted to search for markers of SCA. The study involved collection of blood samples from 240 ICD patients who were then followed for more than 2 years for their arrhythmic outcomes. Resulting data was used for the search of statistical associations between life threatening events and SNPs.

FIG. 3 is a statistical plot of Single Nucleotide Polymorphisms (“SNPs”).

FIG. 4 is a decision tree based on a recursive partitioning algorithm.

FIGS. 5A and 5B are genomic groupings of MAPP based on the recursive partitioning algorithm.

FIG. 6 is a chromosomal plot of 822 SNPs with p=0.1 for both MAPP and an IDEA-VF study. IDEA-VF was a pilot study to demonstrate the feasibility of collecting blood samples from post Myocardial Infarct (“MI”) patients to search for genetic markers that indicate the patient risk for SCA. Approximately 100 post-MI patients participated in the study and roughly half of them were ICD patients with life threatening arrhythmias and the rest were patients without ICDs.

FIG. 7A represents a listing of SNPs potentially useful as genetic markers based on logical criteria (CART tree).

FIG. 7B represents a listing of SNPs potentially useful as genetic markers based on biological criteria (clustering in genome).

FIG. 7C represents a listing of SNPs potentially useful as genetic markers based on statistical criteria (min radius).

FIG. 8 shows graphically the operation of a genetic screen in conjunction with existing medical tests.

FIG. 9 shows 25 SNPs identified as SCD or SCA-associated SNPs having p-values less than 0.0001 from the analysis of the MAPP data.

FIG. 10 shows the SNPs identified by the MAPP and IDEA-VF studies associated with risk at SCD.

FIG. 11 is a list of rs numbers and corresponding SEQ ID NO.'s.

### DETAILED DESCRIPTION OF THE INVENTION

The invention relates to an isolated nucleic acid molecule useful to predict Sudden Cardiac Death (“SCD”) or Sudden Cardiac Arrest (“SCA”) risk and Single Nucleotide Polymorphism (“SNP”) selected from SEQ ID NO.'s 1-822 that can be used in the diagnosis, distinguishing, and detecting thereof.

### DEFINITIONS

Unless defined otherwise, all technical and scientific terms used herein have the same meaning as commonly understood by one of ordinary skill in the art to which this invention belongs. For purposes of the present invention, the following terms are defined below.

The terms “a”, “an” and “the” include plural referents unless the context clearly dictates otherwise.

The term “isolated” refers to nucleic acid, or a fragment thereof, that has been removed from its natural cellular environment.

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides. The term “nucleic acid” encompasses the terms “oligonucleotide” and “polynucleotide”.

“Probes” or “primers” refer to single-stranded nucleic acid sequences that are complementary to a desired target nucleic acid. The 5′ and 3′ regions flanking the target complement sequence reversibly interact by means of either complementary nucleic acid sequences or by attached members of another affinity pair. Hybridization can occur in a base-specific manner where the primer or probe sequence is not required to be perfectly complementary to all of the sequences of a template. Hence, non-complementary bases or modified bases can be interspersed into the primer or probe, provided that base substitutions do not inhibit hybridization. The nucleic acid template may also include “nonspecific priming sequences” or “nonspecific sequences” to which the primers or probes have varying degrees of complementarity. In certain embodiments, a probe or primer comprises 101 or fewer nucleotides, from about 3 to 101 nucleotides, from about 5 to 85, from about 6 to 75, from about 7 to 60, from about 8 to 50, from about 10 to 45, from about 12 to 30, from about 12 to 25, from about 15 to 20, or from about any number of base pairs flanking the 5′ and 3′ side of a region of interest to sufficiently identify, or result in hybridization. Further, the ranges can be chosen from group A and B where for A: the probe or primer is greater than 5, greater than 10, greater than 15, greater than 20, greater than 25, greater than 30, greater than 40, greater than 50, greater than 60, greater than 70, greater than 80, greater than 90 and greater than 100 base pairs in length. For B, the probe or primer is less than 102, less than 95, less than 90, less than 85, less than 80, less than 75, less than 70, less than 65, less than 60, less than 55, less than 50, less than 45, less than 40, less than 35, less than 30, less than 25, less than 20, less than 15, or less than 10 base pairs in length. In other embodiments, the probe or primer is at least 70% identical to the contiguous nucleic acid sequence or to the complement of the contiguous nucleotide sequence, for example, at least 80% identical, at least 90% identical, at least 95% identical, and is capable of selectively hybridizing to the contiguous nucleic acid sequence or to the complement of the contiguous nucleotide sequence. Preferred primer lengths include 25 to 35, 18 to 30, and 17 to 24 nucleotides. Often, the probe or primer further comprises a label, e.g. radioisotope, fluorescent compound, enzyme, or enzyme co-factor.

To obtain high quality primers, primer length, melting temperature (Tₘ), GC content, specificity, and intra- or inter-primer homology are taken into account in the present invention. You et al., “BatchPrimer3: A high throughput web application for PCR and sequencing primer design”, BMC Bioinformatics 2008, 9:253; Yang X, Scheffler B E, Weston L A, “Recent developments in primer design for DNA polymorphism and mRNA profiling in higher plants”, Plant Methods 2006, 2(1):4. Primer specificity is related to primer length and the final 8 to 10 bases of the 3′ end sequence where a primer length of 18 to 30 bases is one possible embodiment. Abd-Elsalam K A: “Bioinformatics tools and guideline for PCR primer design”, Africa Journal of Biotechnology 2003, 2(5):91-95. Tₘ is closely correlated to primer length, GC content and primer base composition. One possible ideal primer Tₘ is in the range of 50 to 65° C. with GC content in the range of 40 to 60% for standard primer pairs. Dieffenbatch C W, Lowe T M J, Dveksler G S, “General concepts for PCR primer design”, In PCR primer, A Laboratory Manual. Edited by: Dieffenbatch C W, Dveksler G S. New York, Cold Spring Harbor Laboratory Press; 1995:133-155. However, the optimal primer length varies depending on different types of primers. For example, SNP genotyping primers may require a longer primer length of 25 to 35 bases to enhance their specificity, and thus the corresponding Tₘ might be higher than 65° C. Also, a suitable Tₘ can be obtained by setting a broader GC content range (20 to 80%).

The probes or primers can also be variously referred to as antisense nucleic acid molecules, polynucleotides or oligonucleotides, and can be constructed using chemical synthesis and enzymatic ligation reactions known in the art. For example, an antisense nucleic acid molecule (e.g. an antisense oligonucleotide) can be chemically synthesized using naturally occurring nucleotides or variously modified nucleotides designed to increase the biological stability of the molecules or to increase the physical stability of the duplex formed between the antisense and sense nucleic acids. The primers or probes can further be used in Polymerase Chain Reaction (PCR) amplification.

The term “genetic material” refers to a nucleic acid sequence that is sought to be obtained from any number of sources, including without limitation, whole blood, a tissue biopsy, lymph, bone marrow, hair, skin, saliva, buccal swabs, purified samples generally, cultured cells, and lysed cells, and can comprise any number of different compositional components (e.g. DNA, RNA, tRNA, siRNA, mRNA, or various non-coding RNAs). The nucleic acid can be isolated from samples using any of a variety of procedures known in the art. In general, the target nucleic acid will be single stranded, though in some embodiments the nucleic acid can be double stranded, and a single strand can result from denaturation. It will be appreciated that either strand of a double-stranded molecule can serve as a target nucleic acid to be obtained. The nucleic acid sequence can be methylated, non-methylated, or both, and can contain any number of modifications. Further, the nucleic acid sequence can refer to amplification products as well as to the native sequences.

Allele Specific Oligomer (“ASO”) refers to a primary oligonucleotide having a target specific portion and a target-identifying portion, which can query the identity of an allele at a SNP locus. The target specific portion of the ASO of a primary group can hybridize adjacent to the target specific portion and can be made by methods well known to those of ordinary skill.

Bi-allelic and multi-allelic refers to two, or more than two alternate forms of a SNP, respectively, occupying the same locus in a particular chromosome or linkage structure and differing from other alleles of the locus at a polymorphic site.

### Single Nucleotide Polymorphism (“SNP”)

Generally, genetic variations are associated with human phenotypic diversity and sometimes disease susceptibility. As a result, variations in genes may prove useful as markers for disease or other disorder or condition. Variation at a particular genomic location is due to a mutation event in the conserved human genome sequence, leading to two possible nucleotide variants at that genetic locus. If both nucleotide variants are found in at least 1% of the population, that location is defined as a Single Nucleotide Polymorphism (“SNP”). Moreover, SNPs in close proximity to one another are often inherited together in blocks called haplotypes. One phenomenon of SNPs is that they can undergo linkage disequilibrium, which refers to the tendency of specific alleles at different genomic locations to occur together more frequently than would be expected by random change. Alleles at given loci are said to be in complete equilibrium if the frequency of any particular set of alleles (or haplotype) is the product of their individual population frequencies. Several statistical measures can be used to quantify this relationship. Devlin and Risch 1995 Sep. 20; 29(2):311-22).

With respect to alleles, a more common nucleotide is known as the major allele and the less common nucleotide is known as the minor allele. An allele found to have a higher than expected prevalence among individuals positive for a given outcome is considered a risk allele for that outcome. An allele found to have a lower than expected prevalence among individuals positive for an outcome is considered a protective allele for that outcome. But while the human genome harbors 10 million “common” SNPs, minor alleles indicative of heart disease are often only shared by as little as one percent of a population.

Hence, as provided herein, certain SNPs found by one or a combination of these methods have been found useful as genetic markers for risk-stratification of SCD or SCA in individuals. Genome-wide association studies are used to identify disease susceptibility genes for common diseases and involve scanning thousands of samples, either as case-control cohorts or in family trios, utilizing hundreds of thousands of SNP markers located throughout the human genome. Algorithms can then be applied that compare the frequencies of single SNP alleles, genotypes, or multi-marker haplotypes between disease and control cohorts. Regions (loci) with statistically significant differences in allele or genotype frequencies between cases and controls, pointing to their role in disease, are then analyzed. For example, following the completion of a whole genome analysis of patient samples, SNPs for use as clinical markers can be identified by any, or combination, of the following three methods:

(1) Statistical SNP Selection Method: Univariate or multivariate analysis of the data is carried out to determine the correlation between the SNPs and the study outcome, life threatening arrhythmias for the present invention. SNPs that yield low-p values are considered as markers. These techniques can be expanded by the use of other statistical methods such as linear regression.

(2) Logical SNP Selection Method: Clustering algorithms are used to segregate the SNP markers into categories which would ultimately correlate with the patient outcomes. Classification and Regression Tree (“CART”) is one of the clustering algorithms that can be used. In that case, SNPs forming the branching nodes of the tree will be the markers of interest.

(3) Biological SNP Selection Method: SNP markers are chosen based on the biological effect of the SNP, as it might affect the function of various proteins. For example, a SNP located on a transcribed or a regulatory portion of a gene that is involved in ion channel formation would be good candidates. Similarly, a group of SNPs that are shown to be located closely on the genome would also hint the importance of the region and would constitute a set of markers.

Genetic markers are non-invasive, cost-effective and conducive to mass screening of individuals. The SNPs identified herein can be effectively used alone or in combination with other SNPs as well as with other clinical markers for risk-stratification/assessment and diagnosis of SCD, or SCA. Further, these genetic markers in combination with other clinical markers for SCA are effectively used for identification and implantation of ICDs in individuals at risk for SCA. The genetic markers taught herein provide greater specificity and sensitivity in identification of individuals at risk.

### Sudden Cardiac Arrest (“SCA”)

SCA, also known as, Sudden Cardiac Death (“SCD”) results from an abrupt loss of heart function. It is commonly brought on by an abnormal heart rhythm. Sudden cardiac death occurs, within a short time period, generally less than an hour from the onset of symptoms. Despite recent progress in the management of cardiovascular disorders generally, and cardiac arrhythmias in particular, SCA, remains both a problem for the practicing clinician and a major public health issue.

In the United States, SCA accounts for approximately 325,000 deaths per year. More deaths are attributable to SCA than to lung cancer, breast cancer, or AIDS. This represents an incidence of 0.1-0.2% per year in the adult population. Myerburg, R J et al., “Cardiac arrest and sudden cardiac death”, In Braunwald E, ed.: A Textbook of Cardiovascular Medicine. 6^{th} ed. Philadelphia: Saunders; W B., 2001: 890-931 and American Cancer Society. Cancer Facts and Figures 2003: 4, Center for Disease Control 2004.

In 60% to 80% of cases, SCA occurs in the setting of Coronary Artery Disease (“CAD”). Most instances involve Ventricular Tachycardias (“VT”) degenerating to Ventricular Fibrillation (“VF”) and subsequent asystole. Fibrillation occurs when transient neural triggers impinge upon an unstable heart causing normally organized electrical activity in the heart to become disorganized and chaotic. Complete cardiac dysfunction results. Non-ischemic cardiomyopathy and infiltrative, inflammatory, and acquired valvular diseases account for most other SCA, or SCD, events. A small percentage of SCAs occur in the setting of ion channel mutations responsible for inherited abnormalities such as the long/short QT syndromes, Brugada syndrome, and catecholaminergic ventricular tachycardia. These conditions account for a small number of SCAs. In addition, other genetic abnormalities such as hypertrophic cardiomyopathy and congenital heart defects such as anomalous coronary arteries are responsible for SCA.

Currently, five arrhythmia markers are often used for risk assessment in Myocardial Infarction (“MI”) patients: (1) Heart Rate (“HR”) Variability, (2) severe ventricular arrhythmia, (3) signal averaged Electro Cardio Gram (“ECG”), (4) left ventricular Ejection Fraction (“EF”) and (5) electrophysiology (“EP”) (studies). Table 1 illustrates the mean sensitivity and specificity values for each of these five arrhythmia markers. As shown, these markers have relatively high specificity values, but low sensitivity values.

TABLE 1

| | | Severe | | Left | |
|---|---|---|---|---|---|
| | HR | Ventricular | Signal | Ventricular | |
| | Variability | Arrhythmia | Averaged | Ejection | Electrophysiology |
| Test | on AECG | on AECG | ECG | Fraction (EF) | (EP) Studies |
| Sensitivity | 49.8% | 42.8% | 62.4% | 59.1% | 61.8% |
| Specificity | 85.8% | 81.2% | 77.4% | 77.8% | 84.1% |

The most commonly used marker, EF, has a sensitivity of 59%, meaning that 41% of the patients would be missed if EF were the only marker used. Although EP studies provide slightly better indications, they are not performed very frequently due to their rather invasive nature. Hence, the identification of patients who have a propensity toward SCA remains as an unmet medical need.

ECG parameters indicative of SCA, or SCD, are QRS duration, late potentials, QT dispersion, T-wave morphology, Heart rate variability and T-wave alternans. Electrical alternans is a pattern of variation in the shape of the ECG waveform that appears on an every-other-beat basis. In humans, alternation in ventricular repolarization, namely, repolarization alternans, has been associated with increased vulnerability to ventricular tachycardia/ventricular fibrillation and sudden cardiac death. Pham, Q., et al., “T-wave alternans: marker, mechanism, and methodology for predicting sudden cardiac death. Journal of Electrocardiology”, 36: 75-81. Analysis of the morphology of an ECG (i.e., T-wave alternans and QT interval dispersion) has been recognized as means for assessing cardiac vulnerability.

Certain biological factors are predictive of risk for SCA such as a previous clinical event, ambient arrhythmias, cardiac response to direct stimulations, and patient demographics. Similarly, analysis of heart rate variability has been proposed as a means for assessing autonomic nervous system activity, the neural basis for cardiac vulnerability. Heart rate variability, a measure of beat-to-beat variations of sinus-initiated RR intervals, with its Fourier transform-derived parameters, is blunted in patients at risk for SCD. Bigger, JT. “Heart rate variability and sudden cardiac death”, In: Zipes D P, Jalife J, eds. Cardiac Electrophysiology: From Cell to Bedside. Philadelphia, Pa.: WB Saunders; 1999.

Patient history is helpful to analyze the risk of SCA, or SCD. For example, in patients with ventricular tachycardia after myocardial infarction, on the basis of clinical history, the following four variables identify patients at increased risk of sudden cardiac death: (1) syncope at the time of the first documented episode of arrhythmia, (2) New York Heart Association (“NYHA”) Classification class III or IV, (3) ventricular tachycardia/fibrillation occurring early after myocardial infarction (3 days to 2 months), and (4) history of previous myocardial infarctions. Unfortunately, most of these clinical indicators lack sufficient sensitivity, specificity, and predictive accuracy to pinpoint the patient at risk for SCA, with a degree of accuracy that would permit using a specific therapeutic intervention before an actual event.

For example, the disadvantage of focusing solely on ejection fraction is that many patients whose ejection fractions exceed commonly used cut offs still experience sudden death or cardiac arrest. Since EF is not specific in predicting mode of death, decision making for the implantation of an ICD solely on ejection fraction will not be optimal. Buxton, A E et al., “Risk stratification for sudden death: do we need anything more than ejection fraction?” Card. Electrophysiology Rev. 2003; 7: 434-7. Although, electrophysiological (“EP”) studies provide slightly better indication, they are not performed very frequently due to their invasive nature and high cost.

Conventional methods for assessing vulnerability to SCA, or SCD, often rely on power spectral analysis (Fourier analysis) of the cardiac electrogram. However, the power spectrum lacks the ability to track many of the rapid arrhythmogenic changes which characterize T-wave alternans, dispersions and heart rate variability. As a result, a non-invasive diagnostic method of predicting vulnerability to SCA, or SCD, by the analysis of ECG has not achieved wide spread clinical acceptance.

Similarly, both, baroflex sensitivity and heart rate variability, judge autonomic modulation at the sinus node, which is taken as a surrogate for autonomic actions at the ventricular level. Autonomic effects at the sinus node and ventricle can easily be dissociated experimentally and may possibly be a cause of false-positive or false-negative test results. Zipes, D P et al., “Sudden Cardiac Death”; Circulation. 1998; 98:2334-2351.

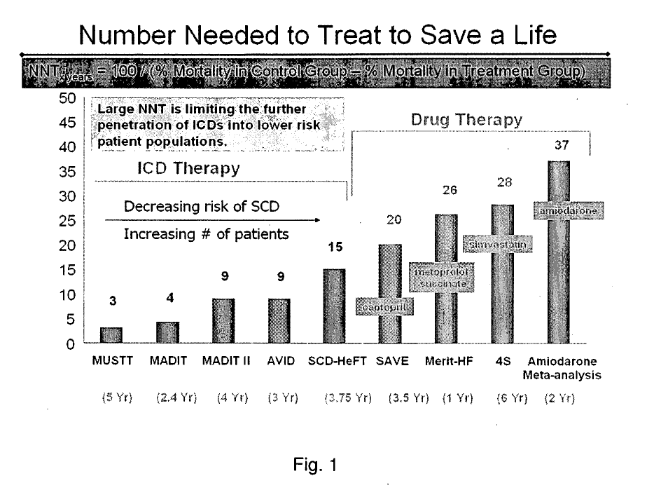

Moreover, as shown in FIG. 1, an increase in the Number Needed to Treat (“NNT”) has been observed for the ICD therapy as the devices are implanted in patients with lower risks. NNT is an epidemiological measure used in assessing the effectiveness of a health-care intervention. The NNT is the number of patients who need to be treated in order to prevent a single negative outcome. In the case of ICDs, currently, devices must be implanted in approximately 17 patients to prevent one death. The other 16 patients may not experience a life threatening arrhythmia and may not receive a treatment. Reduction of the NNT for ICDs would yield to better patient identification methods and allow delivery of therapies to individuals who need them. As a result, it is believed that the need for risk stratification of patients might increase over time as the ICDs are implanted in patients who are generally considered to be at lower risk categories. The net result of the lack of more specific markers for life threatening arrhythmias is the presence of a population of patients who would benefit from ICD therapy, but are not currently indicated, and a subgroup of patients who receive ICD implants, but may not benefit from them.

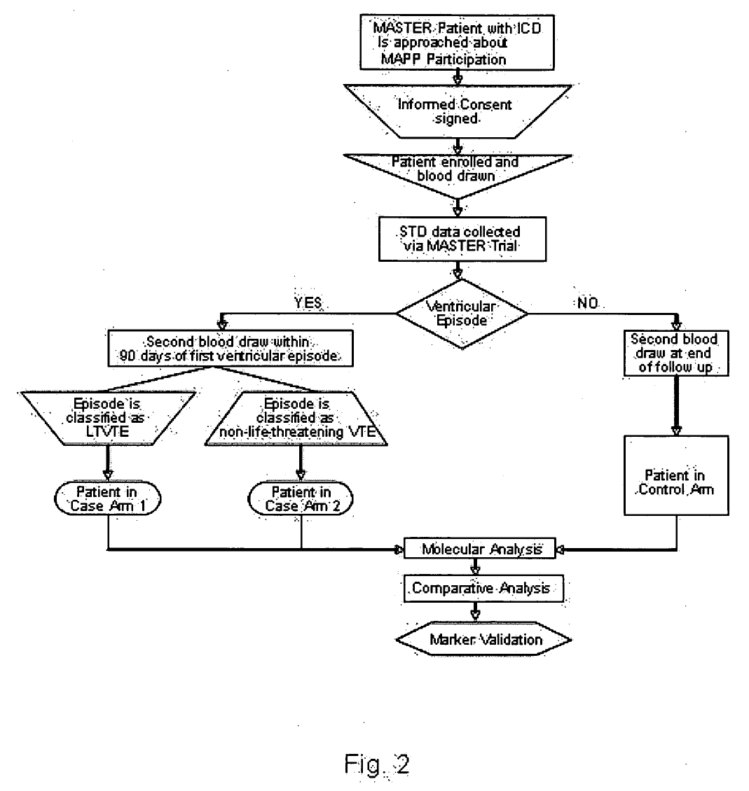

Therefore, in order to identify genetic markers associated with SCA, or SCD, a sub-study (also referred to herein as “MAPP”) to an ongoing clinical trial (also referred to herein as “MASTER”) was designed and implemented. The MASTER study was undertaken to determine the utility of T-wave-alternans test for the prediction of SCA in patients who have had a heart attack and are in heart failure. The overall aim of the study was to assist in identification of patients most likely to benefit from receiving an ICD. Resulting data was used for the search of statistical associations between life threatening events and SNPs. FIG. 2 is a graphical representation of the study design. All patients participating in the MAPP study had defibrillators (ICD) implanted at enrollment and they were followed up for an average of 2.6 years following the ICD implantation. Based on the arrhythmic events that the patients had during this follow-up, they were categorized in three groups as shown in Table 2.

TABLE 2
Outcome of MAPP Patients

| Patient Category | Number |
|---|---|
| CASE 1 - Life Threatening Left Ventricular Event | 33 |
| CASE 2 - Non-life Threatening Left Ventricular Events | 2 |
| CONTROL - No Events | 205 |
| Total | 240 |

Table 3 provides a brief summary of the demographic and physiologic variables that were recorded at the time of enrollment. Except for the Ejection Fraction (“EF”), none of the variables were found to be predictive of the patient outcome, as shown by the large p-values in Table 3. Although the EF gave a p-value less than 0.05, indicating a correlation with the presence of arrhythmic events, it did not provide a sufficient separation of the two groups to act as a prognostic predictor for individual patients, which in turn further confirmed the initial assessment that there is no strong predictor for SCA.

TABLE 3
Demographic and Physiologic Variable Summary For the MAPP
Patient Population

| Variable | Entire MAPP | Case 1 | Control | |
|---|---|---|---|---|
| Name | N = 240 | N = 33 | N = 205 | p-value |
| | Mean (SD) | | | |
| Age (years) | 63.2 (11.0) | 61.6 (8.5) | 63.5 (11.3) | 0.3694 |
| EF (%) | 27.1 (6.5) | 25.0 (6.3) | 27.5 (6.4) | 0.0449 |
| NYHA Class | 2.7 (1.4) | 2.9 (1.4) | 2.7 (1.4) | 0.4015 |
| QRS Width | 115.4 (29.8) | 115.0 (23.8) | 115.5 (30.7) | 0.9443 |
| (msec) | | | | |
| | N (%) | | | |
| Sex (Male) | 209 (87.1) | 26 (78.8) | 183 (88.4) | 0.1582 |
| MTWA | 77 (32.2) | 13 (39.4) | 64 (31.0) | 0.4223 |
| (Negative) | | | | |
| Race | 224 (93.3) | 31 (93.9) | 193 (93.2) | 1 |
| (Caucasian) | | | | |

(EF: Ejection fraction; NYHC: New York Heart Class; MTWA: Microvolt T-Wave Alternans test)

Association of genetic variation and disease can be a function of many factors, including, but not limited to, the frequency of the risk allele or genotype, the relative risk conferred by the disease-associated allele or genotype, the correlation between the genotyped marker and the risk allele, sample size, disease prevalence, and genetic heterogeneity of the sample population. In order to search for associations between SNPs and patient outcomes, genomic DNA was isolated from the blood samples collected from the 240 patients who participated in this study. Following the DNA isolation, a whole genome scan consisting of 317,503 SNPs was conducted using Illumina 300K HapMap gene chips. For each locus, two nucleic acid reads were done from each patient, representing the nucleotide variants on two chromosomes, except for the loci chromosomes on male patients. Four letter symbols were used to represent the nucleotides that were read: cytosine (C), guanine (G), adenine (A), and thymine (T). The structure of the various alleles is described by any one of the nucleotide symbols of Table 4.

TABLE 4
Allele Key used in Sequence Listings

| Nucleotide symbol | Full Name |
|---|---|
| R | Guanine/Adenine (purine) |
| Y | Cytosine/Thymine (pyrimidine) |
| K | Guanine/Thymine |
| M | Adenine/Cytosine |
| S | Guanine/Cytosine |
| W | Adenine/Thymine |
| B | Guanine/Thymine/Cytosine |
| D | Guanine/Adenine/Thymine |
| H | Adenine/Cytosine/Thymine |
| V | Guanine/Cytosine/Adenine |
| N | Adenine/Guanine/Cytosine/Thymine |

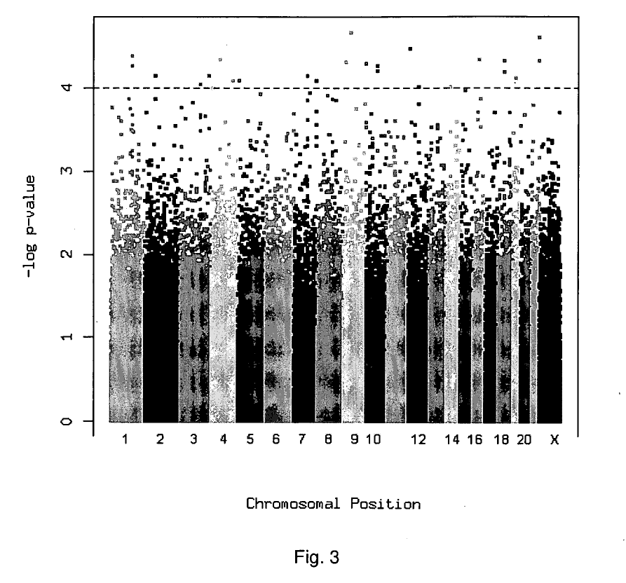

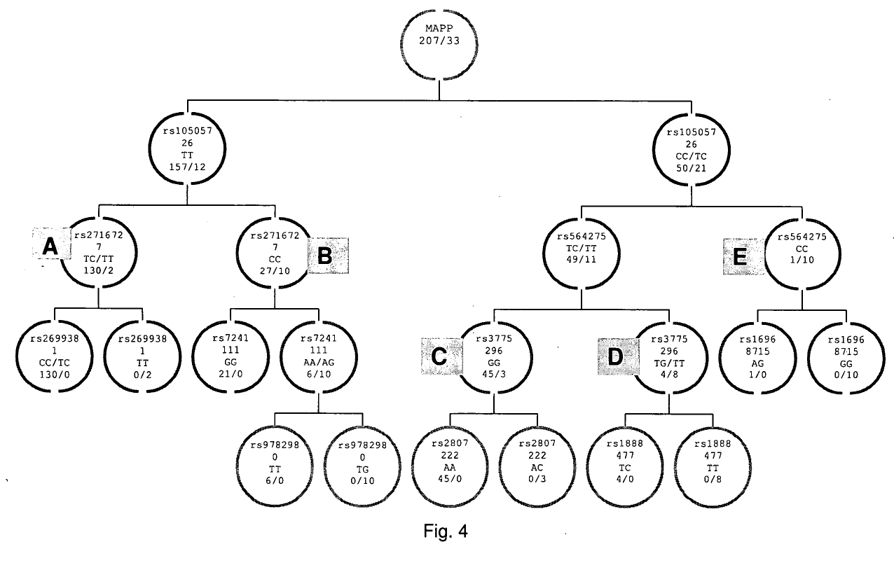

Following the compilation of the genetic data into an electronic database, statistical analysis was carried out. Results from this analysis are provided in FIG. 3. As shown in FIG. 3, a statistical plot of SNPs: p-values graphed as a function of chromosomal position. The dotted line corresponds to a p-value of 0.0001. There were 25 SNPs found in this analysis with a p-value at or less than 0.0001. The y-axis is the negative base 10 logarithm of the p-value. The x-axis is the chromosome and chromosomal position of each SNP on the Illumina gene chip for which a chromosomal location could be determined (N=314,635).

For each SNP, Fisher exact test p-value was calculated. Fisher's exact test is a statistical significance test used in the analysis of categorical data where sample sizes are small. For 2 by 2 tables, the null of conditional independence is equivalent to the hypothesis that the odds ratio equals one. ‘Exact’ inference can be based on observing that in general, given all marginal totals are fixed, the first element of the contingency table has a non-central hypergeometric distribution with non-centrality parameter given by the odds ratio (Fisher, 1935). The alternative for a one-sided test is based on the odds ratio, so alternative=“greater” is a test of the odds ratio being bigger than one.

For a 2×2 contingency table

| | |
|---|---|
| a | C |
| b | D |

the probability of the observed table is calculated by the hypergeometric distribution formula

$$p = ( \begin{matrix}a + b \\ a\end{matrix} ) ( \begin{matrix}c + d \\ c\end{matrix} ) / ( \begin{matrix}n \\ a + c\end{matrix} ) = \frac{( a + b ) ! ( c + d ) ! ( a + c ) ! ( b + d ) !}{n ! a ! b ! c ! d !}$$

Two-sided tests are based on the probabilities of the tables, and take as ‘more extreme’ all tables with probabilities less than or equal to that of the observed table, the p-value being the sum of all such probabilities. Simulation is done conditional on the row and column marginals, and works only if the marginals are strictly positive. Fisher, R. A. (1935) “The Logic of Inductive Inference”, Journal of the Royal Statistical Society Series A 98, 39-54.

Statistical analysis of the data continued with the use of a recursive partitioning algorithm. Recursive partitioning is a nonparametric technique that recursively partitions the data up into homogeneous subsets (with regard to the response). A multi-level “tree” is formed by bisecting each subset of patients based on their value of a given predictor variable. This point of bisection is called a “node”. In this analysis, SNPs were the predictors and the three potential genotypes for each SNP (major allele homozygotes, heterozygotes and minor allele homozygotes) were split into two groups, where the heterozygotes were compacted with one of the two homozygote groups. For a prospectively defined response (in this case, whether a patient is a case or control patient) and set of predictors (SNPs), this method recursively splits the data at each node until either the patients at the resulting end nodes are homogeneous with respect to the response or the end nodes contain too few observations. The decision tree is a visual diagram of the results of recursive partitioning, with the topmost nodes indicating the most discriminatory SNP and each node further split into subnodes accordingly. When this algorithm was applied to 317,498 SNPs, at least a subset of the patients in the analysis cohort was successfully genotyped, and the decision tree shown in FIG. 4 resulted. FIG. 4 provides the decision tree resulting from the application of the recursive partitioning algorithm to the SNPs that were found to be correlated with the patient outcomes in the MAPP study. The two numbers shown in each node correspond to the case and the control patients grouped in that node.

Using only the non-shaded decision nodes on the tree shown in FIG. 4, patients can be categorized in five groups as illustrated in Table 5.

TABLE 5
Genomic Grouping of MAPP Patients Based on
the Results of the Recursive Partitioning Algorithm

| Group | Genome | SCD Risk | ICD Recommendation |
|---|---|---|---|
| A | rs10505726 = TT rs2716727 = TC/TT | $\frac{2}{132} = 1.5 \%$ | Do not implant |
| B | rs10505726 = TT rs2716727 = CC | $\frac{10}{37} = 27 \%$ | Implant |
| C | rs10505726 = CC/TC rs564275 = TC/TT rs3775296 = GG | $\frac{3}{48} = 6.3 \%$ | Do not implant |
| D | rs10505726 = CC/TC rs564275 = TC/TT rs3775296 = TG/TT | $\frac{8}{12} = 66.7 \%$ | Implant |
| E | rs10505726 = CC/TC rs564275 = CC | $\frac{10}{11} = 90.1 \%$ | Implant |

The overall specificity and sensitivity of the combined tests described by Groups A through E in Table 5 can be determined by examining the contingency table (Table 6) of the combined test and MAPP patients in Case 1 patients, who experienced a life threatening VT/VF event versus Case 2 and Control patients who did not. It is desirable that the given test should have a high sensitivity and specificity value. Furthermore, it is not acceptable to sacrifice either one of these features to enhance the other. Therefore, values that are high enough to improve the clinical patient selection process, but low enough to be achievable with current research capabilities were chosen as indicative of SCA. The goal is to have 80% sensitivity and 80% specificity, which is met by 84.8% and 84.5%, respectively, based on calculations from the data in Table 6.

TABLE 6
Sensitivity and Specificity of the Combined Tests Enumerated in Table 5,
Based on the Results of the Recursive Partitioning Algorithm

| | | Experienced VT/VF | | |
|---|---|---|---|---|
| | | Yes | No | Total |
| Combined Tests | Implant | A = 28 | B = 32 | 60 |
| | Do not Implant | C = 5 | D = 175 | 180 |
| | Total | 33 | 207 | 240 |

$$\begin{matrix}Sensitivity\_of \_combined \_test = \frac{A}{A + C} = \frac{28}{28 + 5} = 84.8 \% \\ Specificity\_of \_combined \_test = \frac{D}{B + D} = \frac{175}{175 + 32} = 84.5 \%\end{matrix}$$

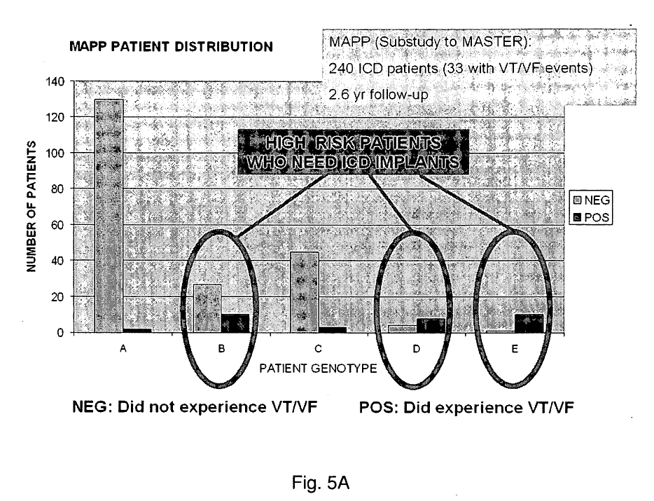

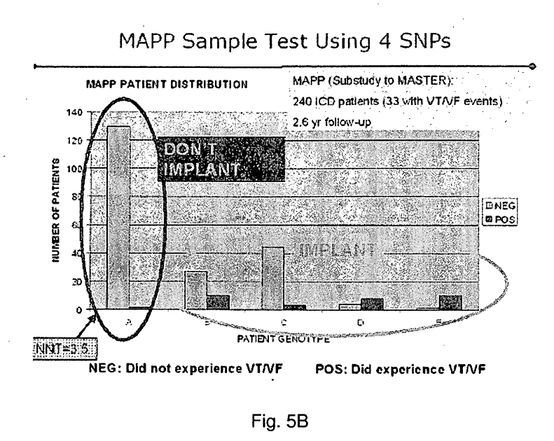

The same results are also shown in the graphical format provided in FIGS. 5A and 5B.

FIGS. 5A and 5B indicates how 4 SNP markers could potentially be used to differentiate patients into high risk and low risk groups. The five SNPs indicated in Table 7 are shown visually among the SNPs in the decision tree in FIG. 4. Group A consists of patients with the TT genotype for rs10505726 and the TC or TT genotype for rs2716727. As indicated by FIG. 5B, these patients would not be considered to be at relatively high risk for a life threatening VT/VF based solely on the genetic diagnostic test. Alternatively, Group B consists of patients with the TT genotype for rs10505726, but with the CC genotype for rs2716727. As indicated by FIG. 5A, these patients would be considered to be at relatively high risk for a life threatening VT/VF based solely on the genetic test and would be considered to be candidates for ICD implantation. Similar logic dictates that Groups D and E are relatively high risk and Group C is relatively low risk for life threatening VT/VF based on the genotypes of rs10505726, rs564275 and rs3775296. Rs7241111 from Table 7 is not used in FIG. 5A, but could be used to further risk stratify the patients.

Additional investigations were conducted using references to determine the nature of the five polymorphisms that were identified by the recursive partitioning algorithm. Results of this work are summarized in Table 7.

TABLE 7
SNPs That Were Found to Be Statistically Significant
Using the Recursive Partitioning Analysis

| | Fisher | | | | | |
|---|---|---|---|---|---|---|
| | Exact Test | Chromosome | Gene | Entrez | Functional | Chromosome |
| SNP | p-value | number | Name | ID | Class | Position |
| rs10505726 | 3.46 × 10⁻⁵ | 12 | PARP11 | 57097 | Intron | 12:3848218 |
| rs2716727 | 3.67 × 10⁻³ | 2 | — | — | — | 2:39807249 |
| rs564275 | 3.72 × 10⁻³ | 9 | GLIS3 | 169792 | Intron | 9:4084320 |
| rs7241111 | 7.33 × 10⁻³ | 18 | — | — | — | 18:63002332 |
| rs3775296 | 6.01 × 10⁻² | 4 | TLR3 | 7098 | Mrna-utr | 4:187234760 |

Persons skilled in the art of medical diagnosis will appreciate that there are multiple methods for the combination of measurements from a patient contemplated by the present invention. For example, a triple test given during pregnancy utilizes the three factors measured from a female subject, and a medical decision is made by further addition of the age of the subject. Similarly, SNPs described in this invention can be combined with other patient information, such as co-morbidities (e.g. diabetes, obesity, cholesterol, family history), parameters derived from electrophysiological measurements such as T-wave alternans, heart rate variability and heart rate turbulence, hemodynamic variables such as ejection fraction and end diastolic left ventricular volume, to yield a superior diagnostic technique. Furthermore, such a combination of a set markers can be achieved by multiple methods, including logical, linear, or non-linear combination of these markers, or by the use of clustering algorithms known in the art.

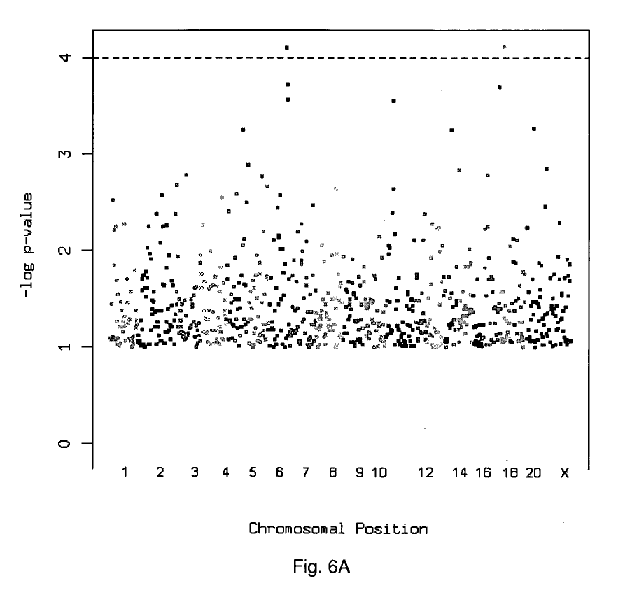

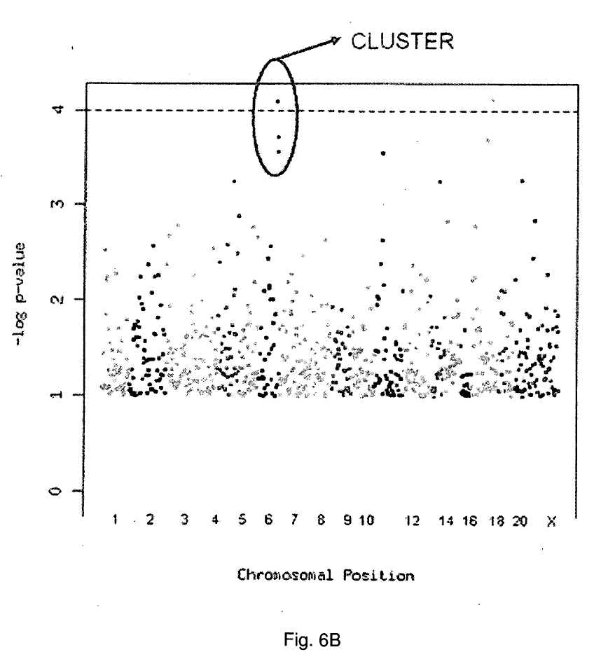

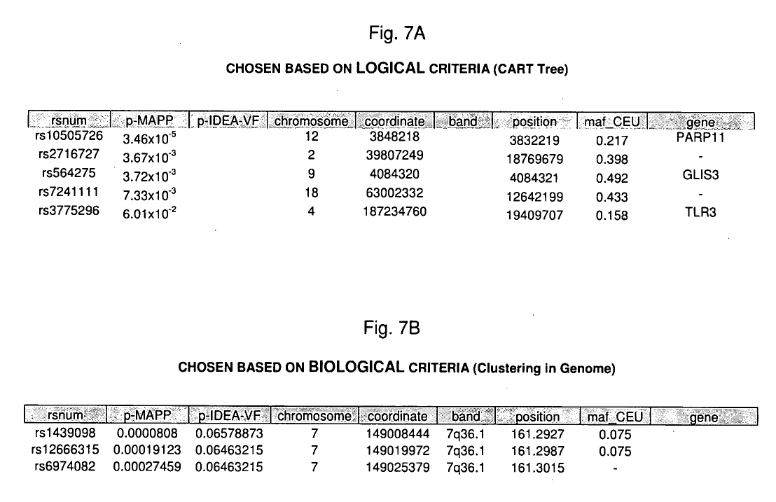

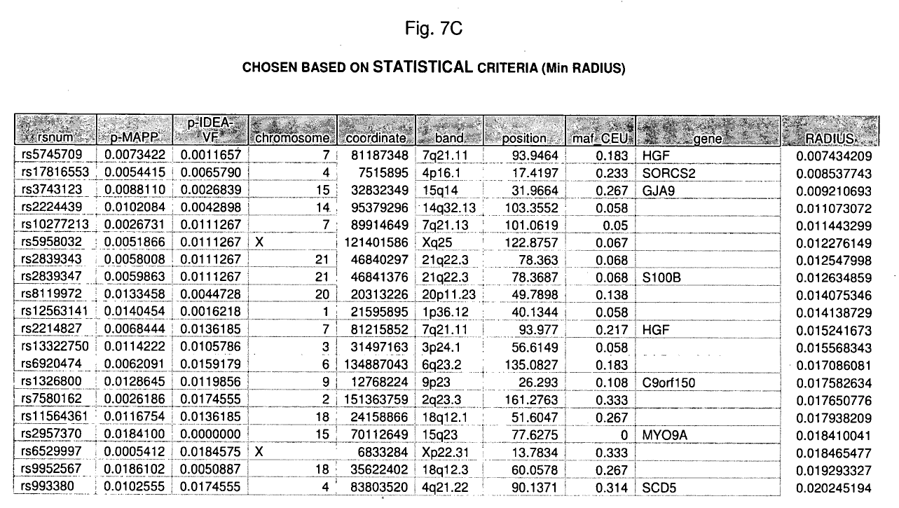

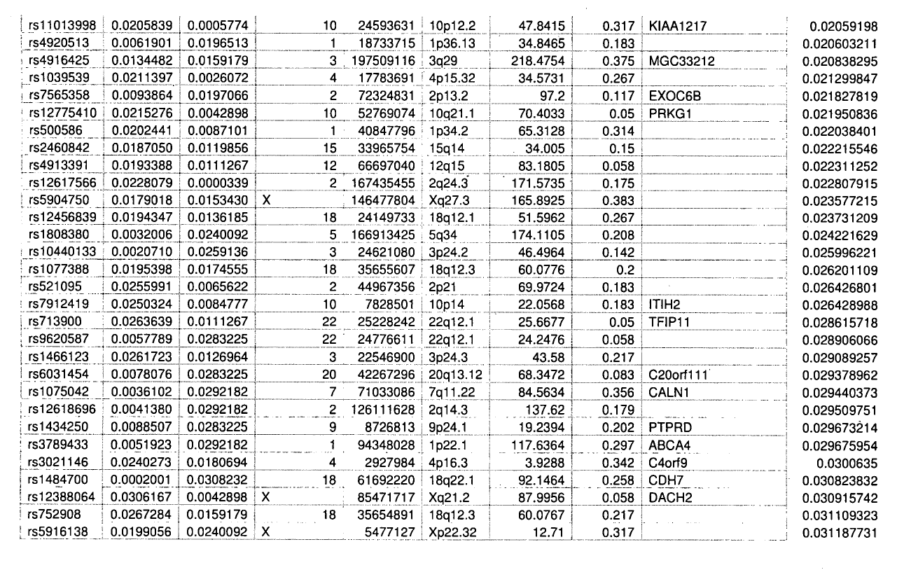

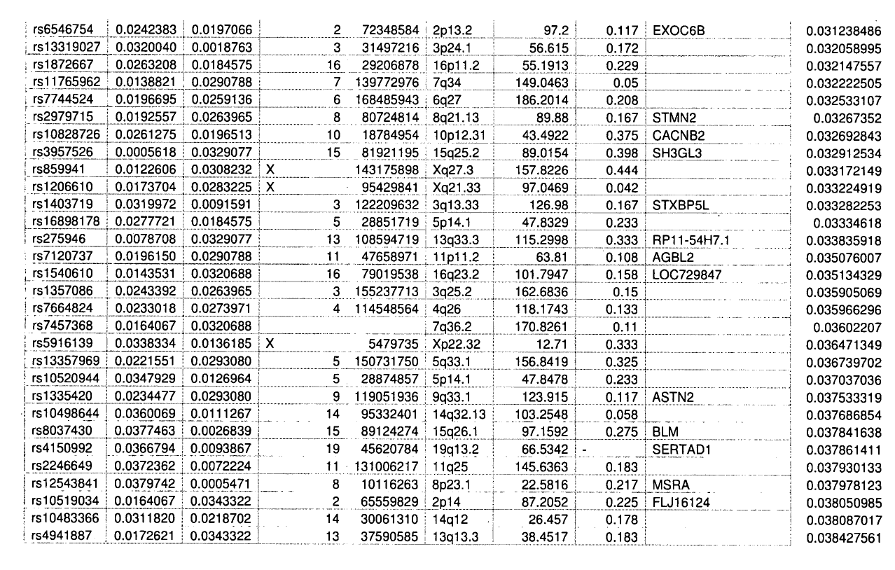

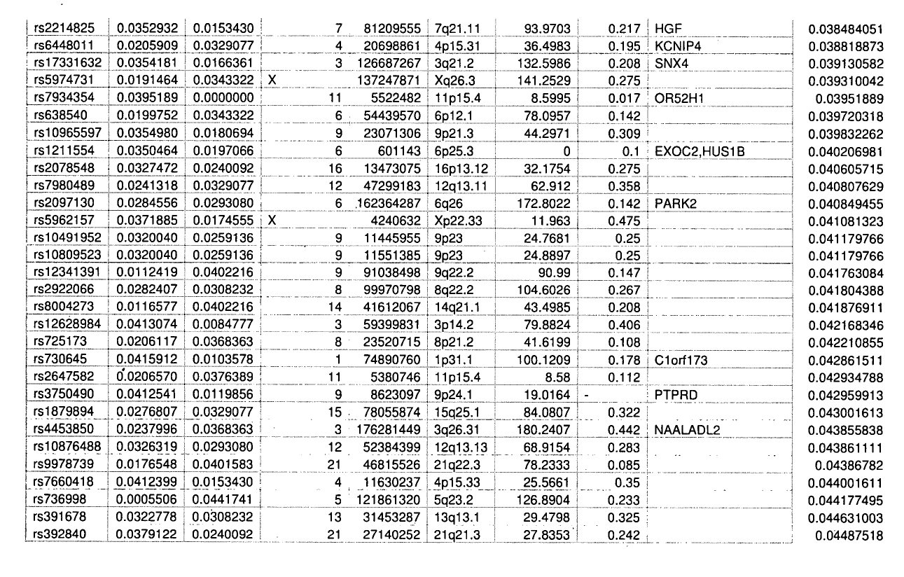

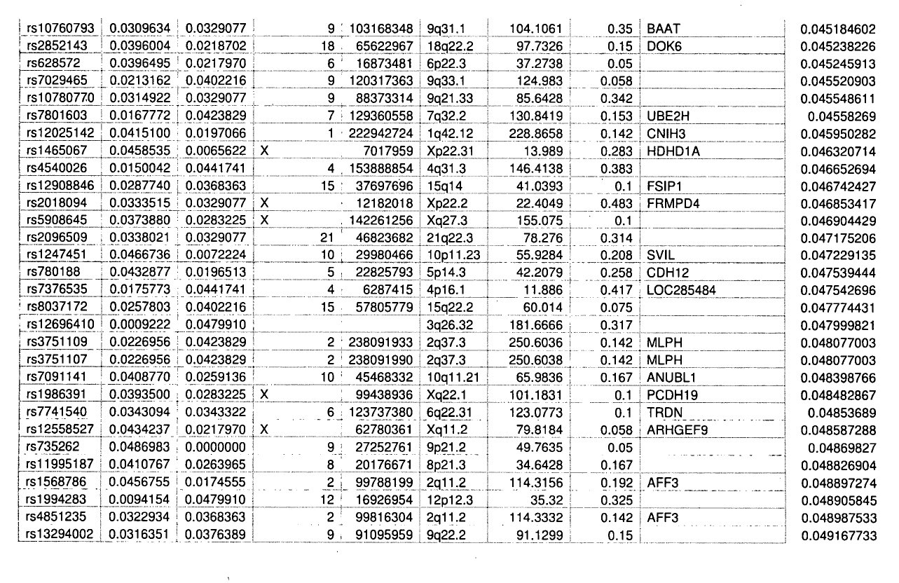

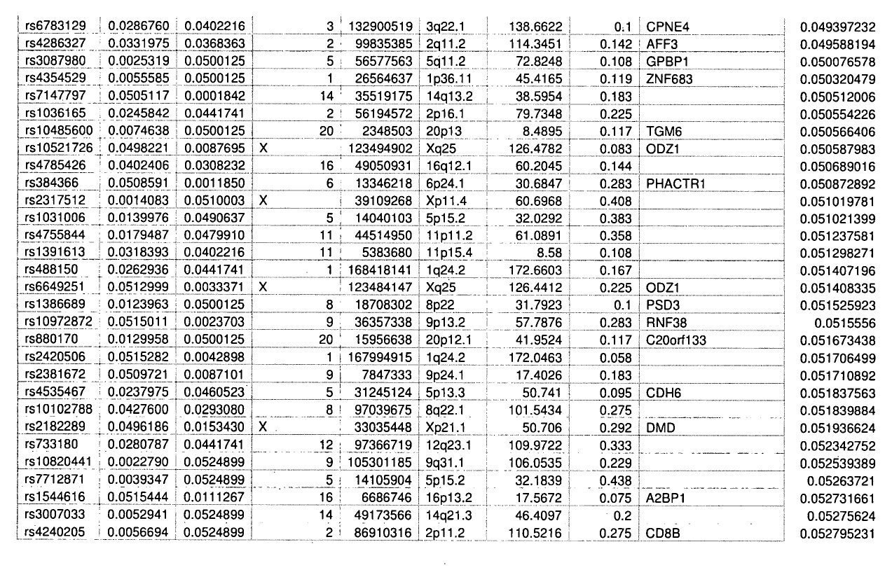

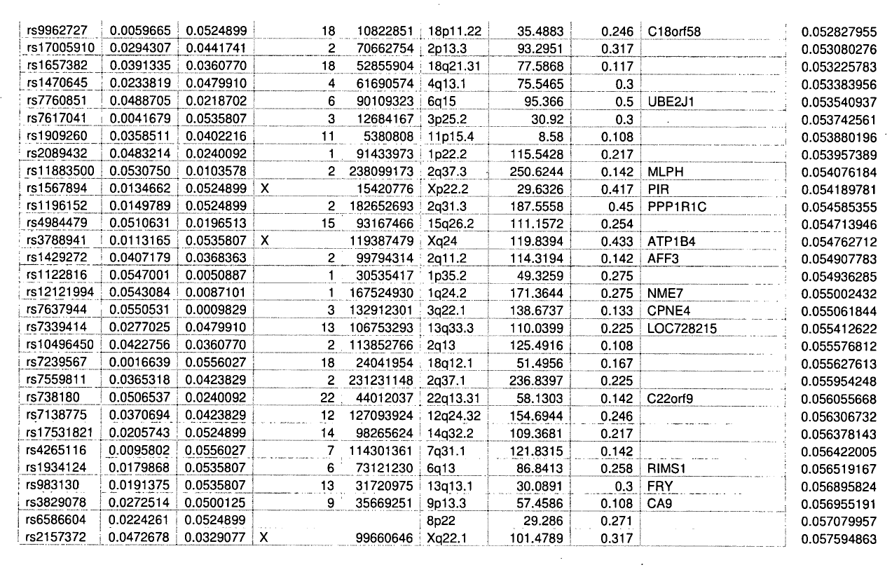

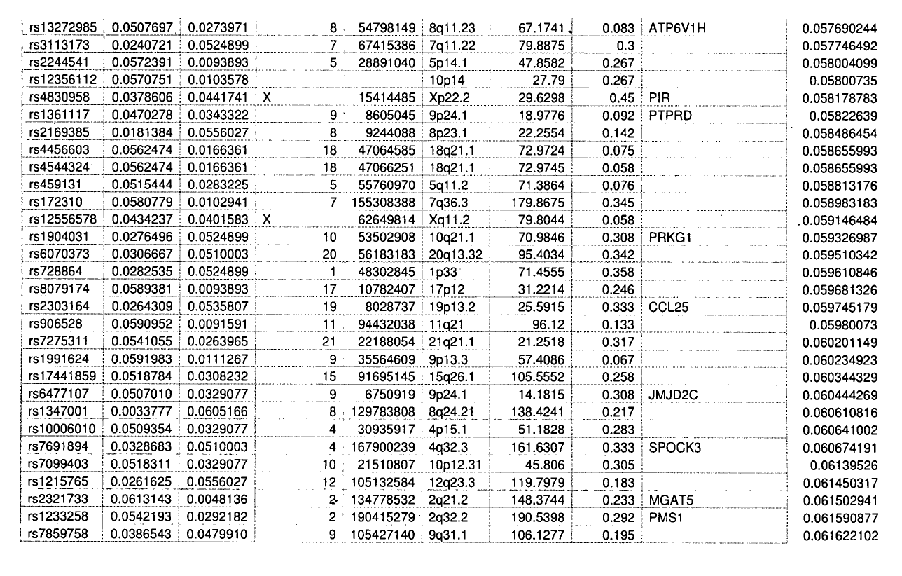

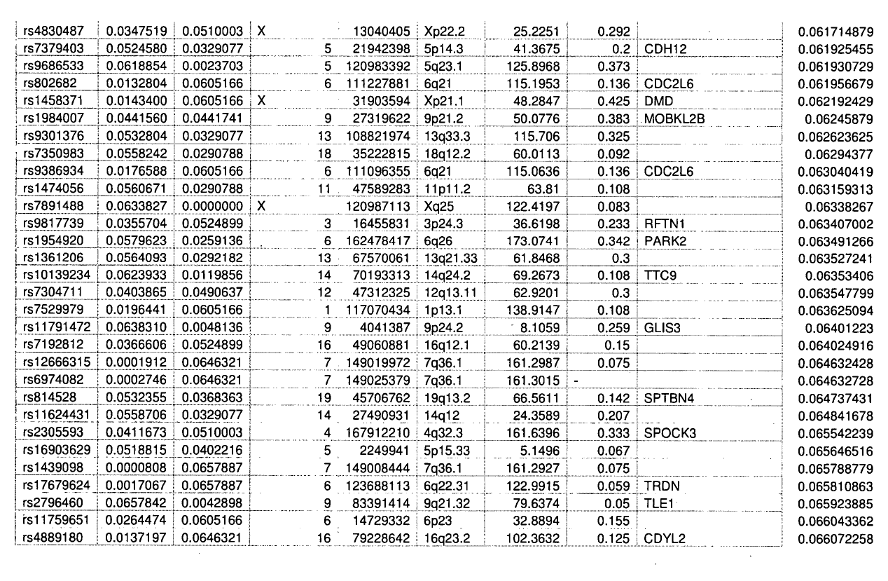

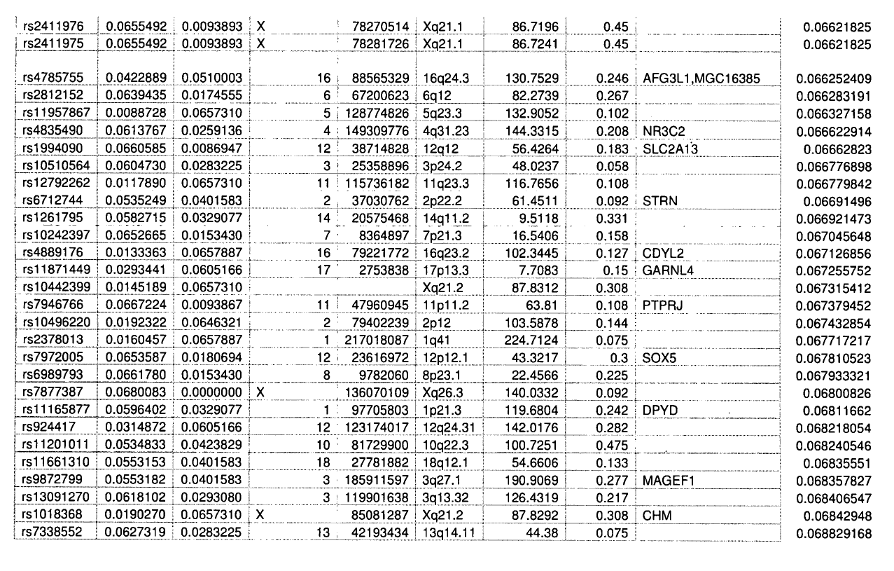

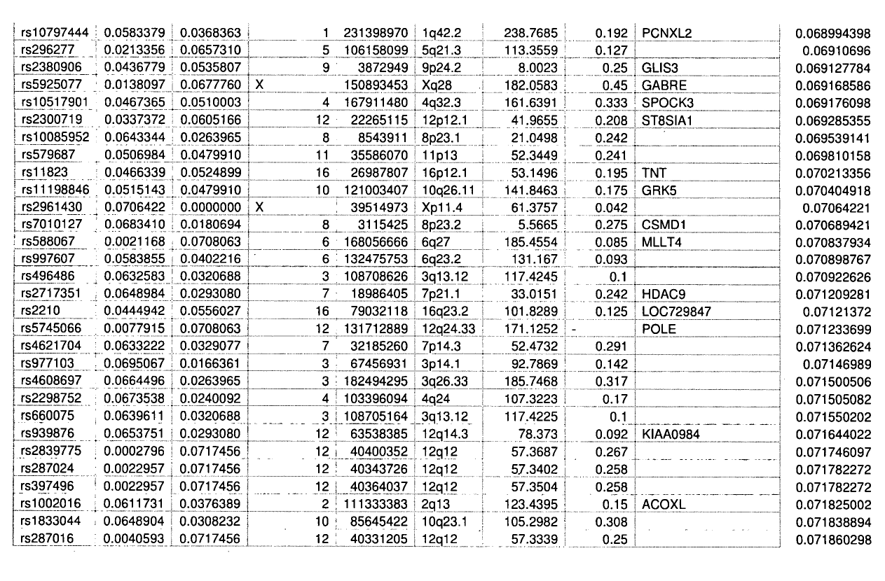

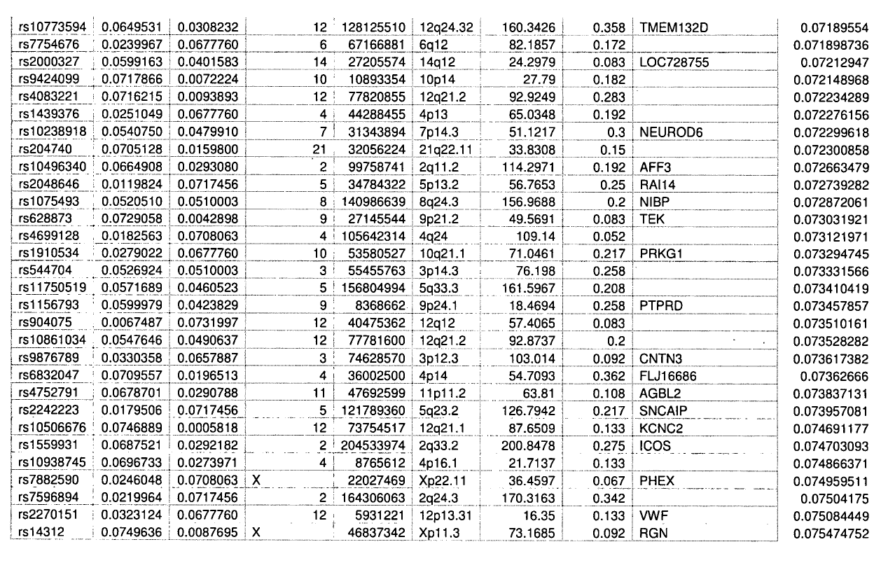

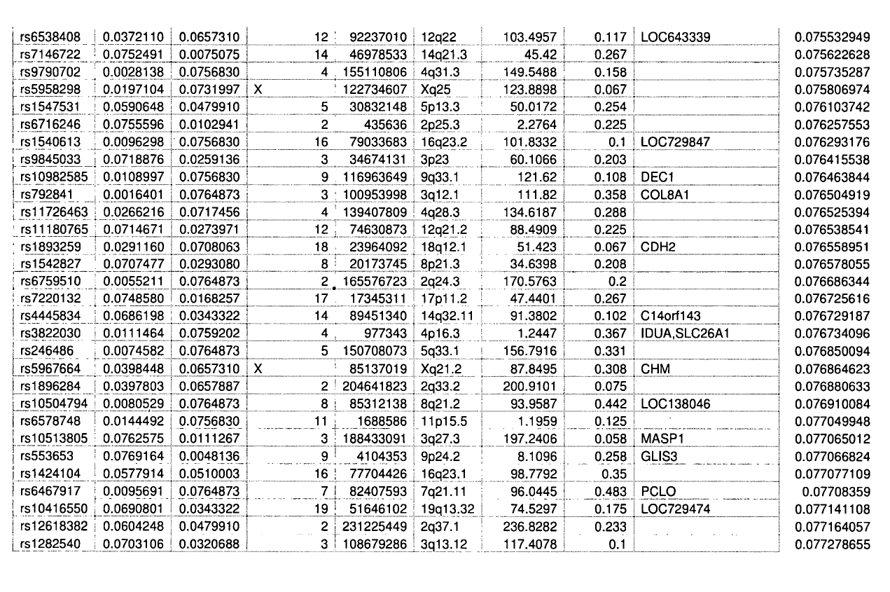

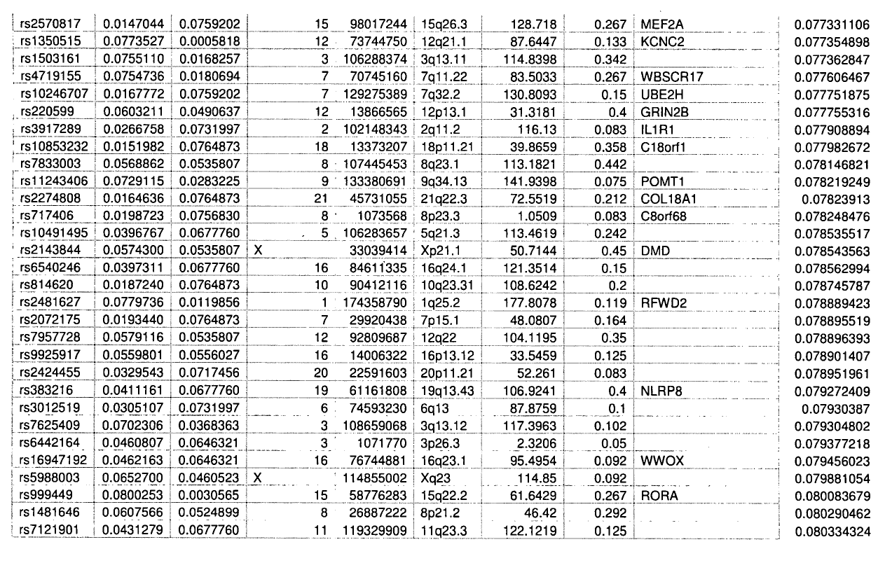

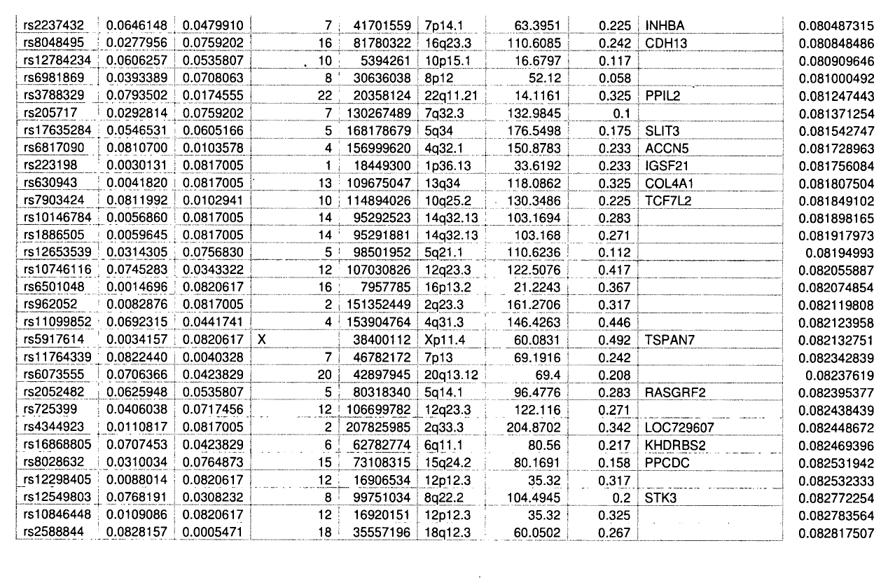

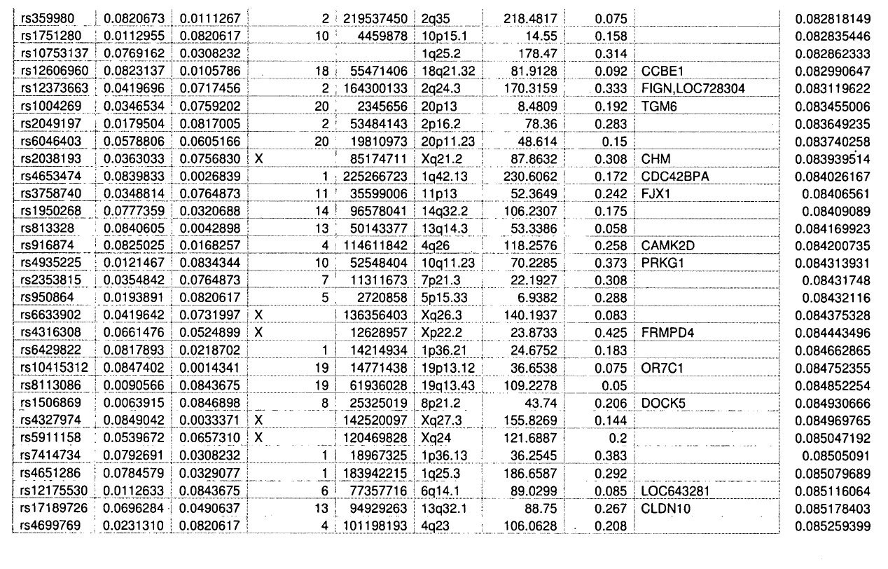

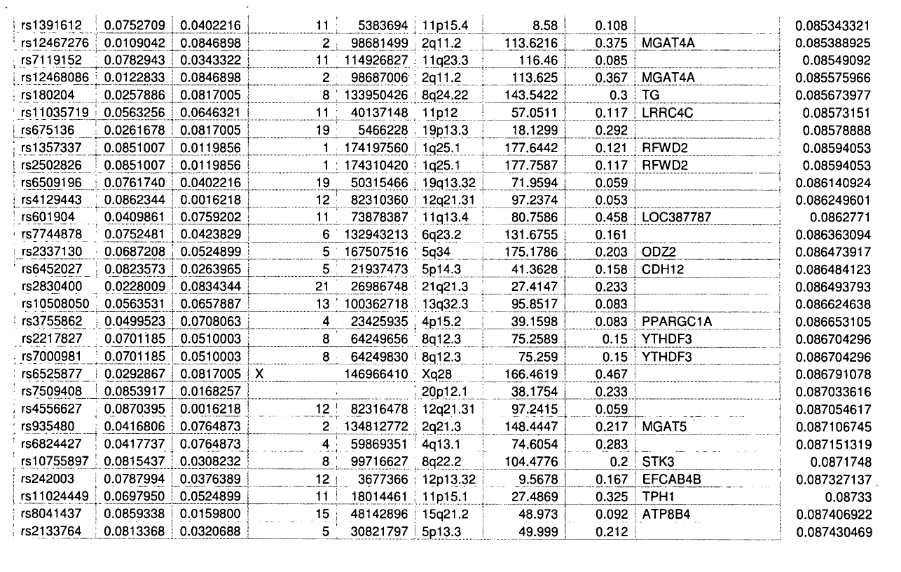

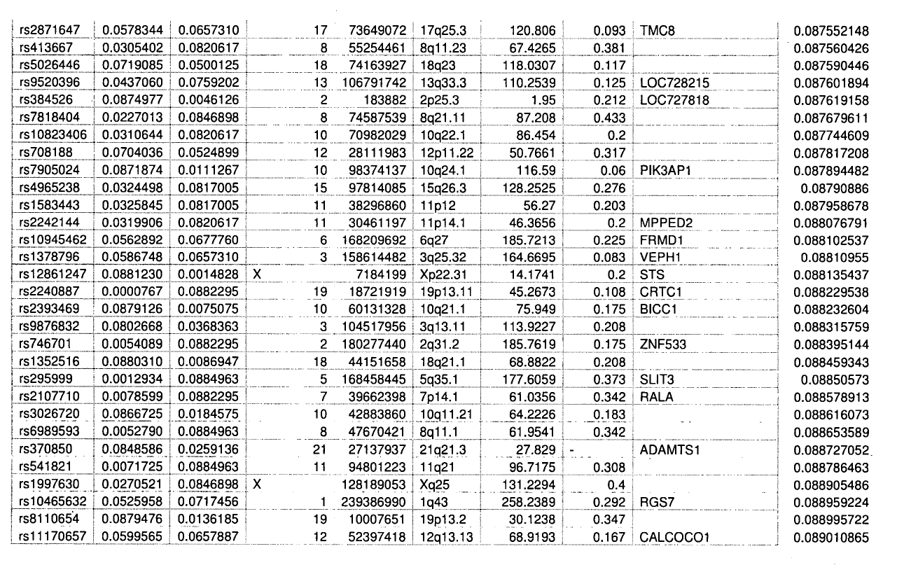

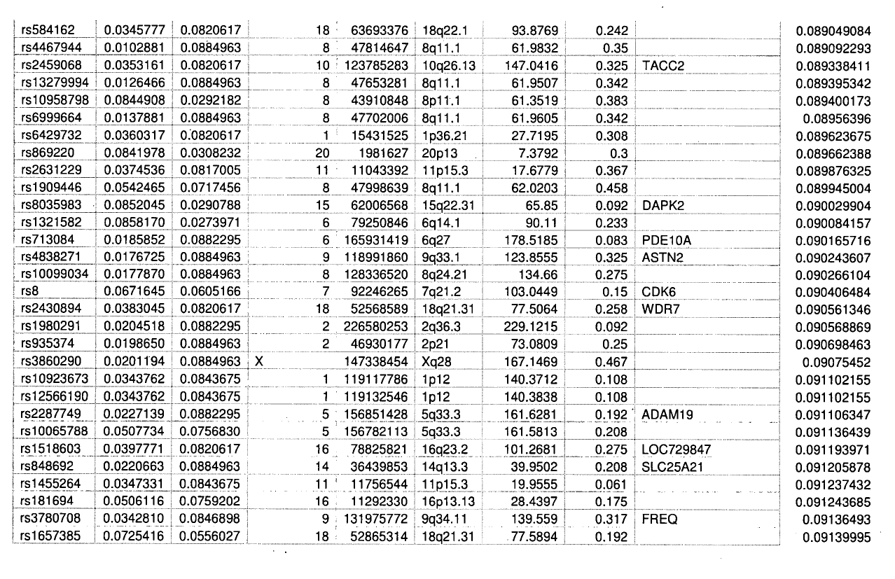

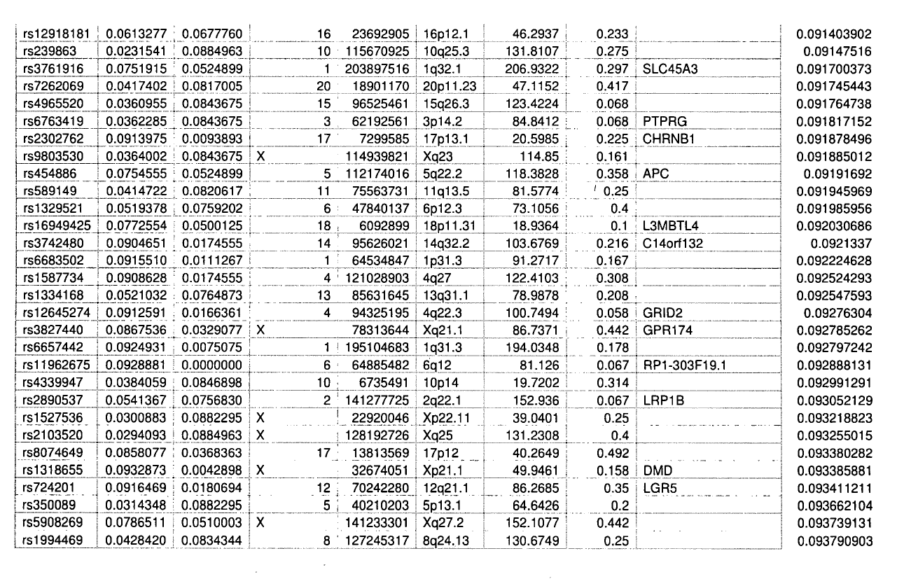

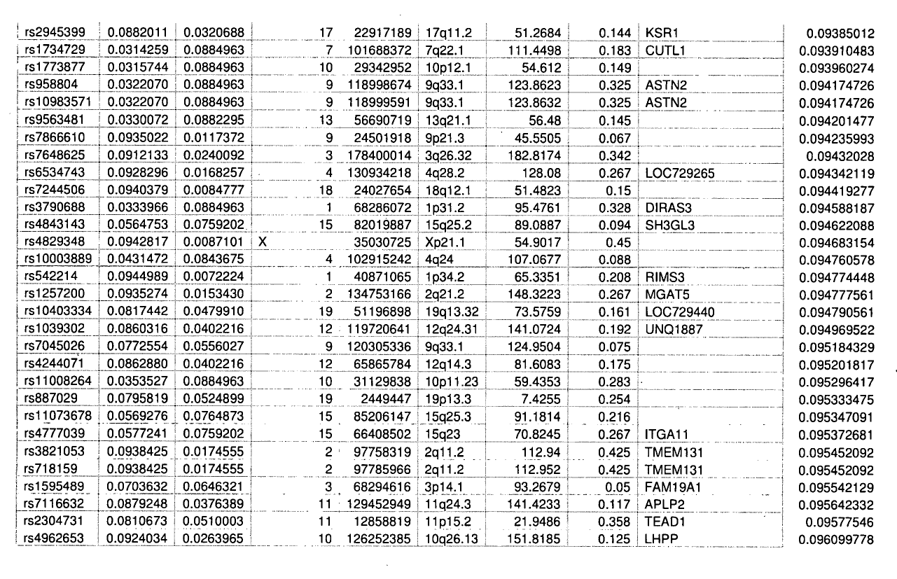

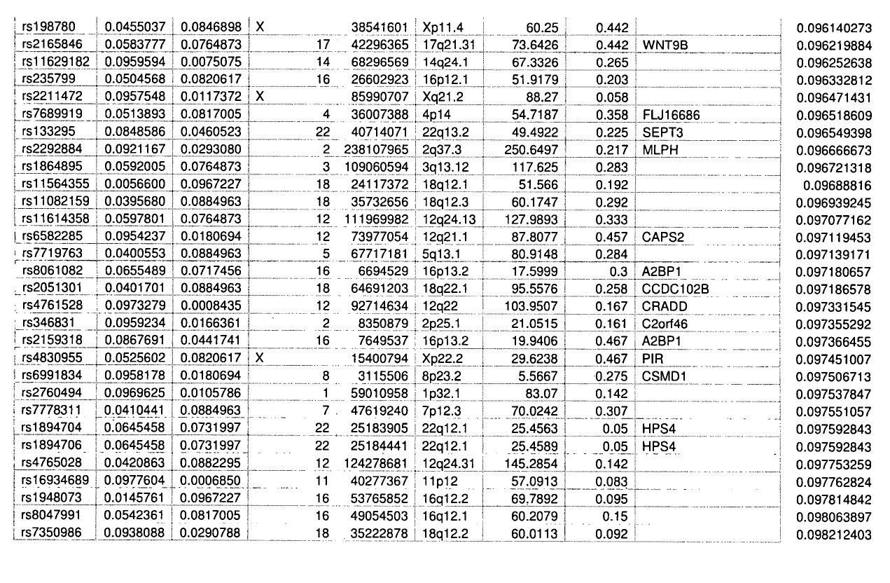

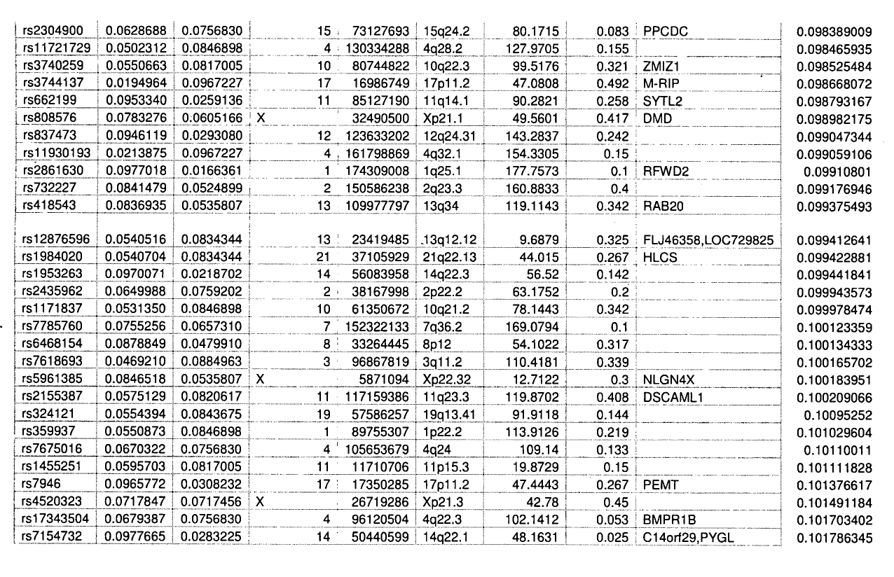

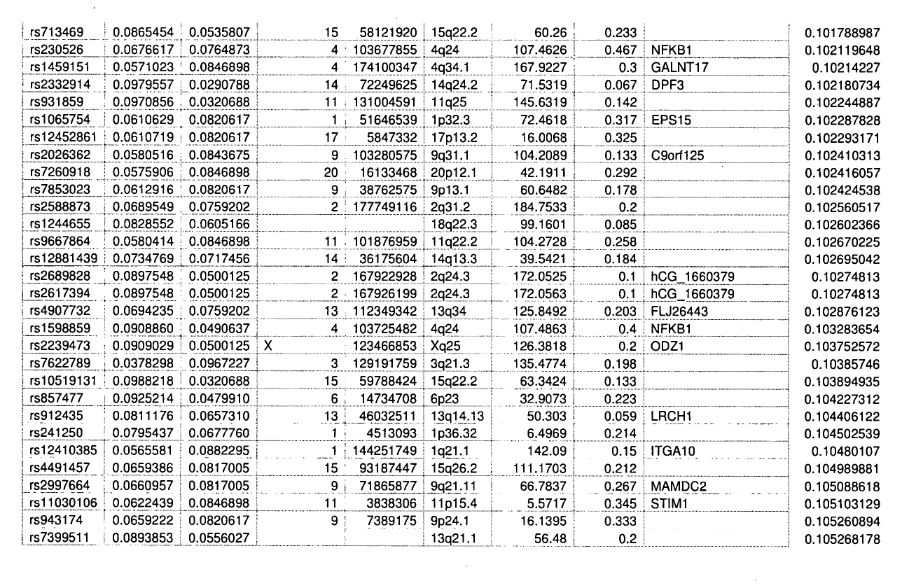

Furthermore, analysis was done using the data obtained from another study, namely the IDEA-VF, where SNP data from 37 ICD and 51 control patients was available. Again, the 317,503 SNPs in the MAPP study were scanned to identify the SNPs with p≦0.1, and 31,008 SNPs were found. These SNPs were tested in the IDEA-VF set, and only 822 of them were found to have p≦0.1, meaning that all 822 SNPs showed p values that were less than 0.1 in two independent studies. The chromosomal plot for these 822 SNPs with p≦0.1 for both MAPP and IDEA-VF are shown in FIG. 6. FIGS. 7A, 7B and 7B contain a detailed table of all the 822 SNPs (SEQ ID NO.'s: 1 to 822) chosen based on logical, biological and statistical criteria. For SEQ ID NO.'s 1-822 of the Sequence Listing of the invention, the SNP is located at position 51.

To determine the presence or absence of an SNP in an individual or patient, an array having nucleotide probes from each of the sequences listed in SEQ ID NO.'s: 1 to 822 can be constructed where each probe is a different nucleotide sequence from 3 to 101 base pairs overlapping the SNP at position 51. In a further embodiment, the sequences of SEQ ID NO.'s: 1 to 822 can be individually used to monitor loss of heterozygosity, identify imprinted genes; genotype polymorphisms, determine allele frequencies in a population, characterize bi-allelic or multi-allelic markers, produce genetic maps, detect linkage disequilibrium, determine allele frequencies, do association studies, analyze genetic variation, or to identify markers linked to a phenotype or, compare genotypes between different individuals or populations.

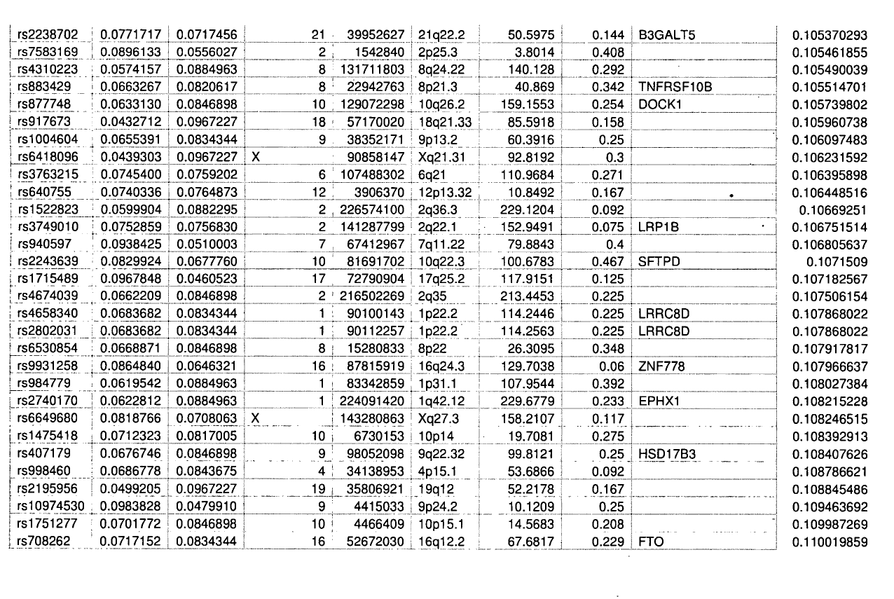

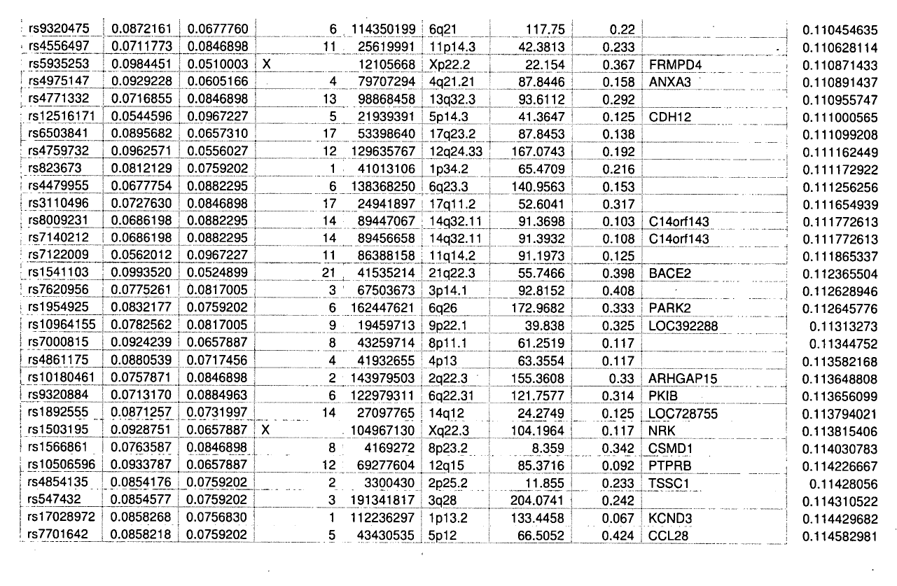

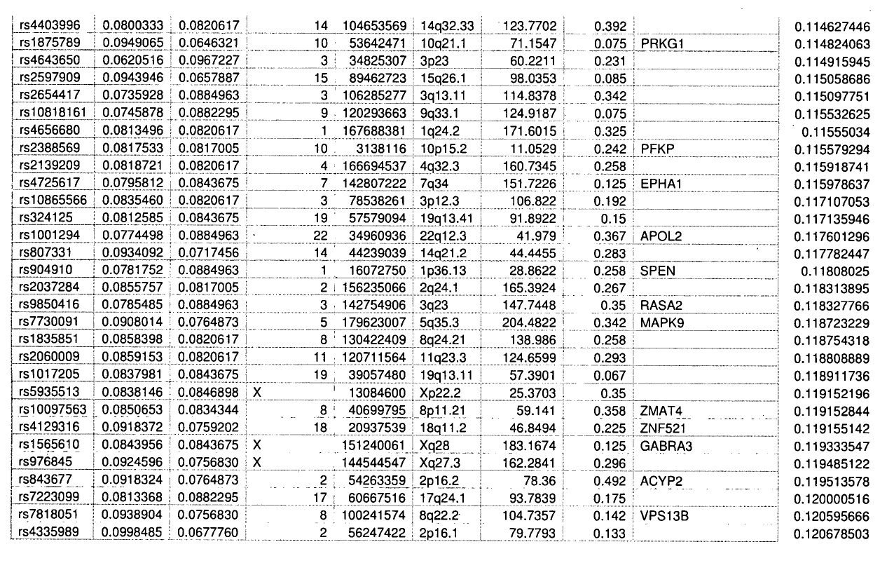

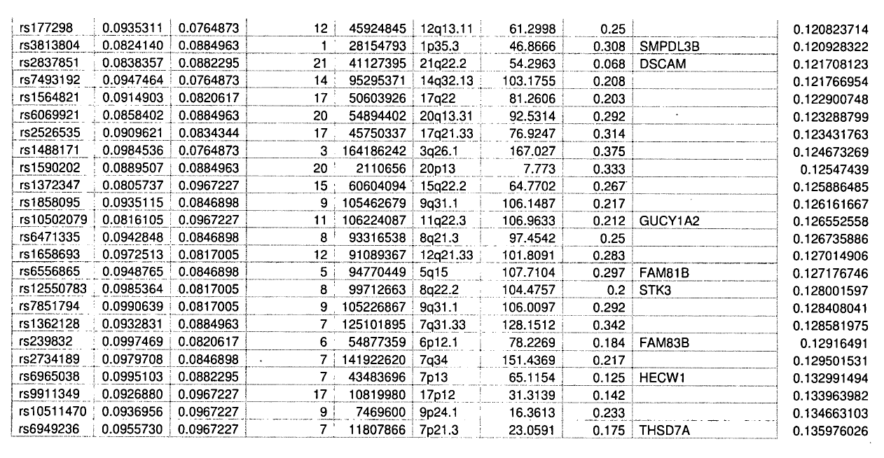

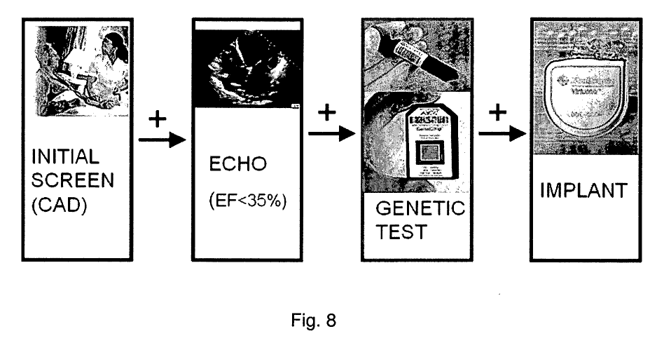

FIG. 8 depicts one embodiment of a clinical utilization of the genetic test created for screening of patients for susceptibility to life threatening arrhythmias. In this embodiment, patients already testing positively for CAD and a low EF would undergo the test for genetic susceptibility using any of the methods described herein. Positive genetic test results would then be used in conjunction with the other test, such as the ones based on the analysis of ECG, and be used to make the ultimate decision of whether or not to implant an ICD.

Patients who are presenting a cardiac condition such as MI are usually subjected to echocardiographic examination to determine the need for an ICD. Based on the present invention, blood samples could also be taken from the patients who have low left ventricular EF. If the genetic tests in combination with the hemodynamic and demographic parameters indicate a high risk for sudden cardiac arrest, then a recommendation is made for an ICD implant. A schematic of this overall process is shown in FIG. 8. A similar recommendation can be made for individuals with no previous history of cardiovascular disease based on a positive genetic screen for one or more of the SNPs taught herein in combination with one or more biological factors including markers, clinical parameters and/or like.

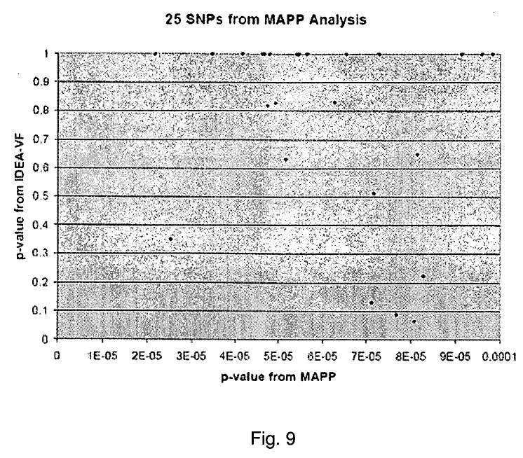

FIG. 9 shows the performance of the genetic markers obtained from the MAPP Study when they were applied to the IDEA-VF patient population. Only the markers with MAPP p values that are less then 0.0001 were tested. As it can be seen from this graph, not all the markers identified as highly significant in MAPP did not give low p-values when they are applied to the IDEA-VF population. A total of 25 SNPs are represented in FIG. 9: rs4878412, rs2839372, rs10505726, rs10919336, rs6828580, rs16952330, rs2060117, rs9983892, rs1500325, rs1679414, rs486427, rs6480311, rs11610690, rs10823151, rs1346964, rs6790359, rs7591633, rs10487115, rs2240887, rs1439098, rs248670, rs4691391, rs2270801, rs12891099, and rs17694397.

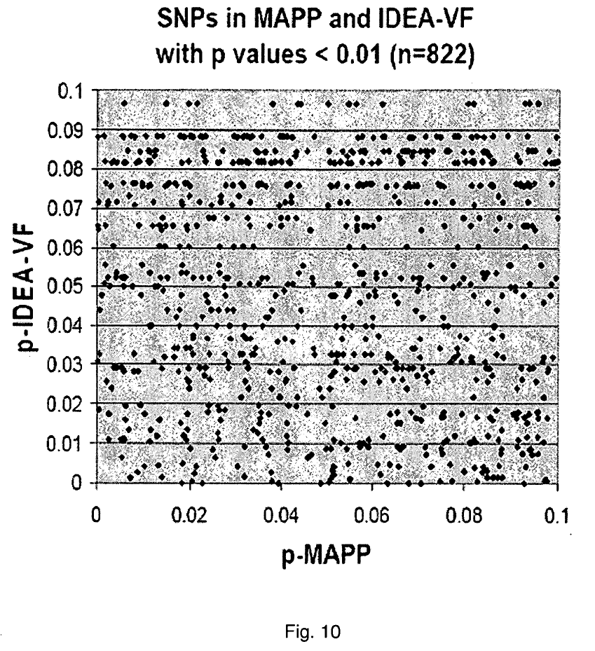

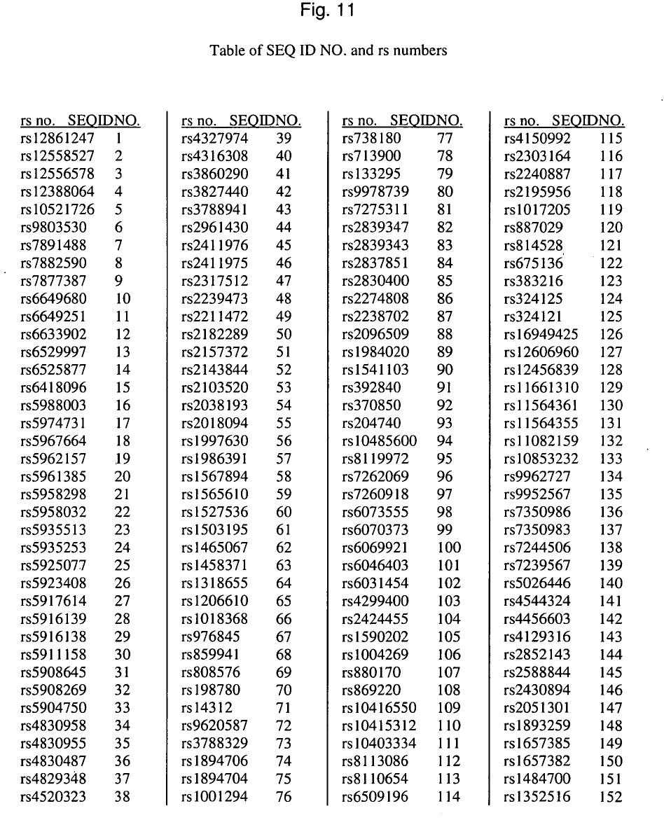

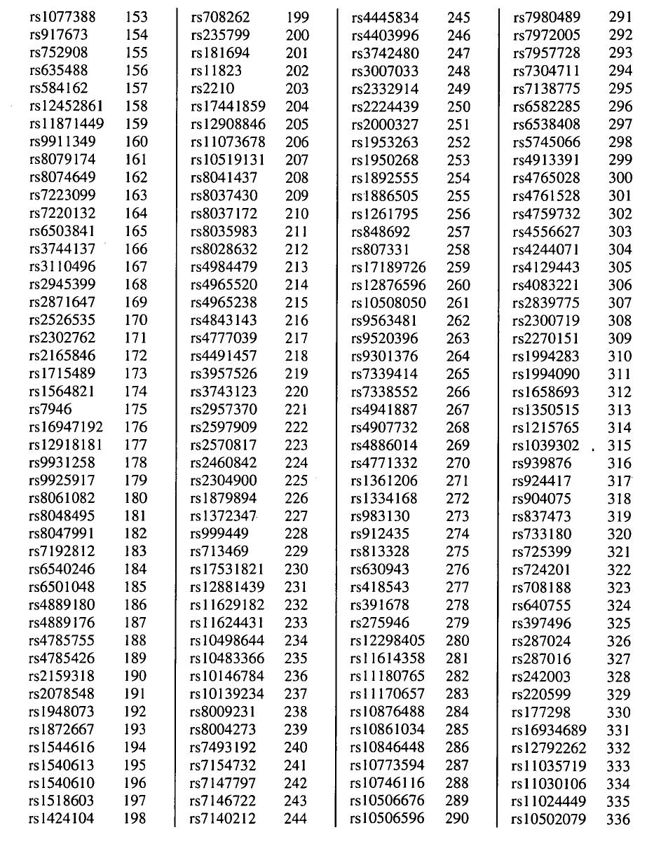

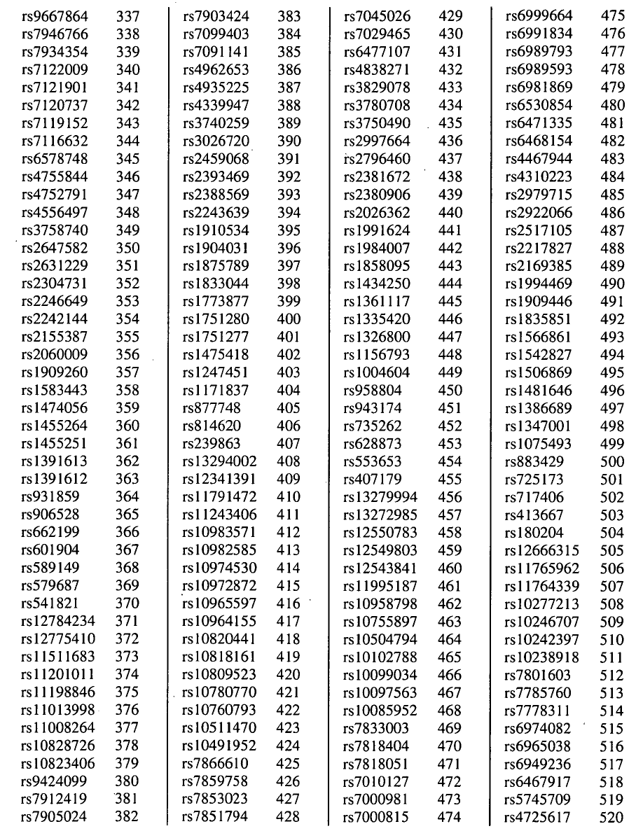

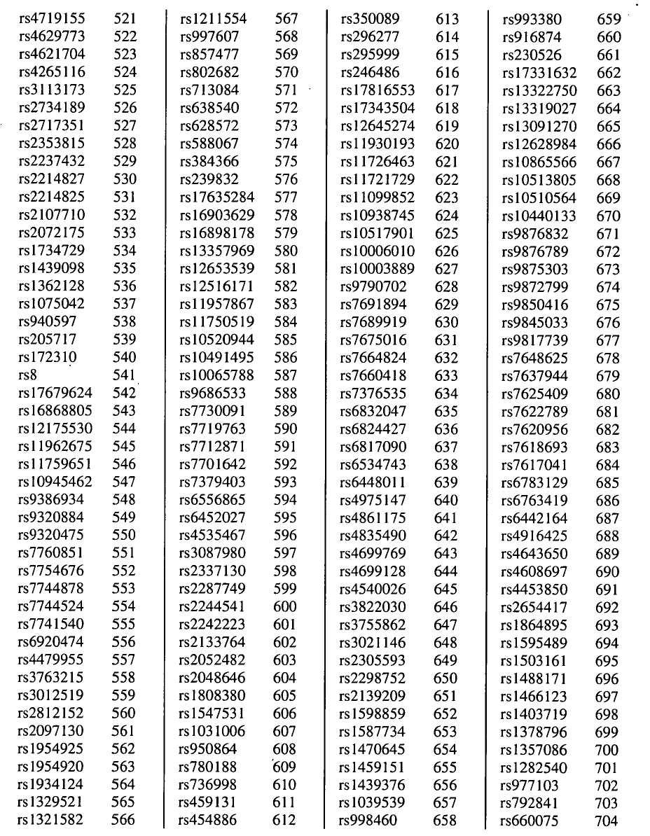

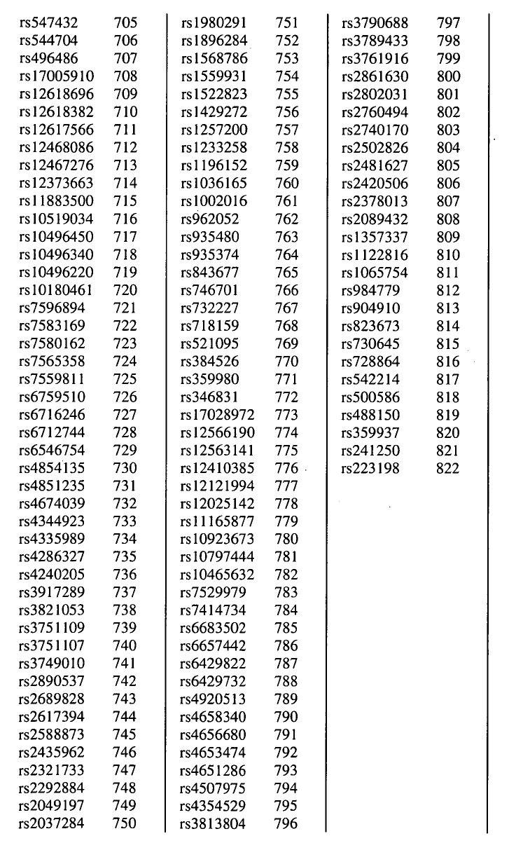

FIG. 10 shows 822 SNPs identified by the MAPP and IDEA-VF studies that are associated with risk of SCA, and is a subset of the total number of 317,503 SNPs scanned from the whole genome using the Illumina 300K HapMap gene chips described herein. FIG. 11 is a list of rs numbers and corresponding SEQ ID NO.'s. Both the rs numbers and the SEQ ID NO.'s can be used interchangeably to identify a particular SNP.

Specific SNPs, either alone or in combination, can be used to predict SCA, or SCD, risk and to select to which drugs or device therapies a patients may be more or less likely to respond. Identification of therapies to which a subject is unlikely to respond allows for quicker access to a more appropriate drug or device therapy. The genetic information can be taken from a biological specimen containing the patient DNA to be used for SNP detection, or from a previously obtained genetic sequence specific to the given patient. Once it is determined that the given patient has a high risk for SCA, then evaluation of possible therapies can be performed. Specific anti-arryhthymic drugs and device therapies including ICD, cardiac resynchronization therapy, anti-tachycardiac pacing therapy and Subcutaneous ICD, or similar therapies can be assessed for their likely effect on the individual patient.

We claim:

1. An isolated nucleic acid molecule useful to predict Sudden Cardiac Arrest (SCA) risk, comprising a nucleotide sequence having a Single Nucleotide Polymorphism (SNP) selected from the group of SEQ ID NO.'s 1-822.

2. The isolated nucleic acid of claim 1, said isolated nucleic acid ranging from about 3 base pairs at positions 50 to 52 in any one of SEQ ID NO.'s 1-822 where position 51 is flanked on either the 5′ and 3′ side by a single base pair, to any number of base pairs flanking the 5′ and 3′ side of position 51.

3. The isolated nucleic acid of claim 2, said isolated nucleic acid being from about 3 to 101 nucleotides in length.

4. The isolated nucleic acid of claim 3, said isolated nucleic acid being a length selected from the group of from about 5 to 101, from about 7 to 101, from about 9 to 101, from about 15 to 101, from about 20 to 101, from about 25 to 101, from about 30 to 101, from about 40 to 101, from about 50 to 101, from about 60 to 101, from about 70 to 101, from about 80 to 101, from about 90 to 101, and from about 99 to 101 nucleotides in length.

5. The isolated nucleic acid molecule of claim 2, being a length selected from the group of 25 to 35, 18 to 30, and 17 to 24 nucleotides

6. The isolated nucleic acid molecule of claim 1, wherein the SNP is selected from the group of rs10505726, rs2716727, rs564275, rs7241111 and rs3775296.

7. The isolated nucleic acid molecule of claim 1, wherein the SNP is selected from the group of rs1439098, rs12666315 and rs6974082.

8. The isolated nucleic acid molecule of claim 1, wherein the SNP is selected from the group of rs4878412, rs2839372, rs10505726, rs10919336, rs6828580, rs16952330, rs2060117, rs9983892, rs1500325, rs1679414, rs486427, rs6480311, rs11610690, rs10823151, rs1346964, rs6790359, rs7591633, rs10487115, rs2240887, rs1439098, rs248670, rs4691391, rs2270801, rs12891099, and rs17694397.

9. The isolated nucleic acid molecule of claim 1, wherein the SNP is bi-allelic.

10. The isolated nucleic acid molecule of claim 1, wherein the SNP is multi-allelic.

11. A polynucleotide useful to predict Sudden Cardiac Arrest (SCA) risk, comprising a complement to a sequence selected from the group of SEQ ID NO.'s 1-822.

12. The polynucleotide of claim 11, said complement ranging from about 3 base pairs at positions 50 to 52 in any one of SEQ ID NO.'s 1-822 where position 51 is flanked on either the 5′ and 3′ side by a single base pair, to any number of base pairs flanking the 5′ and 3′ side of position 51.

13. The polynucleotide of claim 12, said complement being from about 3 to 101 nucleotides in length.

14. The polynucleotide of claim 13, said complement being a length selected from the group of from about 5 to 101, from about 7 to 101, from about 9 to 101, from about 15 to 101, from about 20 to 101, from about 25 to 101, from about 30 to 101, from about 40 to 101, from about 50 to 101, from about 60 to 101, from about 70 to 101, from about 80 to 101, from about 90 to 101, and from about 99 to 101 nucleotides in length.

15. The polynucleotide of claim 12, said complement being a length selected from the group of 25 to 35, 18 to 30, and 17 to 24 nucleotides

16. The polynucleotide of claim 11, having a Single Nucleotide Polymorphism (SNP) selected from the group of rs10505726, rs2716727, rs564275, rs7241111 and rs3775296.

17. The polynucleotide of claim 11, having a Single Nucleotide Polymorphism (SNP) selected from the group of rs1439098, rs12666315 and rs6974082.

18. The polynucleotide of claim 11, wherein the Single Nucleotide Polymorphism (SNP) is selected from the group of rs4878412, rs2839372, rs10505726, rs10919336, rs6828580, rs16952330, rs2060117, rs9983892, rs1500325, rs1679414, rs486427, rs6480311, rs11610690, rs10823151, rs1346964, rs6790359, rs7591633, rs10487115, rs2240887, rs1439098, rs248670, rs4691391, rs2270801, rs12891099, and rs17694397.

19. The polynucleotide of claim 11, having a Single Nucleotide Polymorphism (SNP) wherein the SNP is bi-allelic.

20. The polynucleotide of claim 11, having a Single Nucleotide Polymorphism (SNP) wherein the SNP is multi-allelic.

21. The polynucleotide of claim 11, wherein said complement is an allele-specific probe or primer.

22. An amplified polynucleotide containing a Single Nucleotide Polymorphism (SNP) selected from SEQ ID NO.'s 1-822, or a complement thereof.

23. The amplified polynucleotide of claim 22, said complement ranging from about 3 base pairs at positions 50 to 52 in any one of SEQ ID NO.'s 1-822 where position 51 is flanked on either the 5′ and 3′ side by a single base pair, to any number of base pairs flanking the 5′ and 3′ side of position 51.

24. The amplified polynucleotide of claim 22, said complement being from about 3 to 101 nucleotides in length.

25. A method of distinguishing patients having an increased susceptibility to Sudden Cardiac Arrest (SCA) from patients who do not, comprising the step of detecting at least one Single Nucleotide Polymorphism (SNP) at position 51 in any of SEQ ID NO.'s 1-822 in a nucleic acid sample from said patients, wherein the presence or absence of the SNP can be used to assess increased susceptibility to SCA.

26. The method of distinguishing patients of claim 25, wherein the presence of the SNP is an indication that patients have an increased susceptibility to SCA.

27. The method of distinguishing patients of claim 25, wherein the presence of the SNP is an indication that patients have a decreased susceptibility to SCA.

28. The method of distinguishing patients of claim 25, wherein the SNP is bi-allelic.

29. The method of distinguishing patients of claim 25, wherein the SNP is multi-allelic.

30. The method of distinguishing patients of claim 25, wherein the SNP is selected from the group of rs10505726, rs2716727, rs564275, rs7241111 and rs3775296.

31. The method of distinguishing patients of claim 25, wherein the SNP is selected from the group of rs1439098, rs12666315 and rs6974082.

32. The method of distinguishing patients of claim 25, wherein the SNP is selected from the group of rs4878412, rs2839372, rs10505726, rs10919336, rs6828580, rs16952330, rs2060117, rs9983892, rs1500325, rs1679414, rs486427, rs6480311, rs11610690, rs10823151, rs1346964, rs6790359, rs7591633, rs10487115, rs2240887, rs1439098, rs248670, rs4691391, rs2270801, rs12891099, and rs17694397.

33. The method of distinguishing patients of claim 30, wherein patients having a TT genotype for rs10505726 and a TC or a TT genotype for rs2716727 does not indicate an increased susceptibility to SCA.

34. The method of distinguishing patients of claim 30, wherein patients having a TT genotype for rs10505726 and a CC genotype for rs2716727 indicates an increased susceptibility to SCA.

35. The method of distinguishing patients of claim 30, wherein patients having a CC or TC genotype for rs10505726 and a TC or a TT genotype for rs564275 and a GG genotype for rs3775296 does not indicate an increased susceptibility to SCA.

36. The method of distinguishing patients of claim 30, wherein patients having a CC or TC genotype for rs10505726 and a TC or a TT genotype for rs564275 and a TG and a TT genotype for rs3775296 indicates an increased susceptibility to SCA.

37. The method of distinguishing patients of claim 30, wherein patients having a CC or TC genotype for rs10505726 and a CC genotype for rs564275 indicates an increased susceptibility to SCA.

38. A method of determining Sudden Cardiac Arrest (SCA) risk in a patient, comprising the step of identifying one or more Single Nucleotide Polymorphism (SNP) at position 51 in any of SEQ ID NO.'s 1-822 in a nucleic acid sample from said patient.

39. The method of determining SCA risk of claim 38, wherein the presence of the SNP is an indication that the patient has a risk of SCA.

40. The method of determining SCA risk of claim 38, wherein the presence of the SNP is an indication that the patient does not have a risk of SCA.

41. The method of determining SCA risk of claim 38, wherein the SNP is bi-allelic.

42. The method of determining SCA risk of claim 38, wherein the SNP is multi-allelic.

43. The method of determining SCA risk of claim 38, wherein the SNP is selected from the group of rs10505726, rs2716727, rs564275, rs7241111 and rs3775296.

44. The method of determining SCA risk of claim 38, wherein the SNP is selected from the group of rs1439098, rs12666315 and rs6974082.

45. The method of determining SCA risk of claim 38, wherein the SNP is selected from the group of rs4878412, rs2839372, rs10505726, rs10919336, rs6828580, rs16952330, rs2060117, rs9983892, rs1500325, rs1679414, rs486427, rs6480311, rs11610690, rs10823151, rs1346964, rs6790359, rs7591633, rs10487115, rs2240887, rs1439098, rs248670, rs4691391, rs2270801, rs12891099, and rs17694397.

46. The method of determining SCA risk of claim 43, wherein a patient having a TT genotype for rs10505726 and a TC or a TT genotype for rs2716727 does not indicate a risk of SCA.

47. The method of determining SCA risk of claim 43, wherein a patient having a TT genotype for rs10505726 and a CC genotype for rs2716727 indicates a risk of SCA.

48. The method of determining SCA risk of claim 43, wherein a patient having a CC or TC genotype for rs10505726 and a TC or a TT genotype for rs564275 and a GG genotype for rs3775296 does not indicate a risk of SCA.

49. The method of determining SCA risk of claim 43, wherein a patient having a CC or TC genotype for rs10505726 and a TC or a TT genotype for rs564275 and a TG and a TT genotype for rs3775296 indicates a risk of SCA.

50. The method of determining SCA risk of claim 43, wherein a patient having a CC or TC genotype for rs10505726 and a CC genotype for rs564275 indicates a risk of SCA.

51. A method of determining the need for an Implantable Cardio Defibrillators (ICD), comprising the step of identifying one or more Single Nucleotide Polymorphism (SNP) at position 51 in any of SEQ ID NO.'s 1-822 in a nucleic acid sample from a patient.

52. The method of determining the need for an ICD of claim 51, wherein the presence of the SNP is an indication that the patient has a need for the ICD.

53. The method of determining the need for an ICD of claim 51, wherein the presence of the SNP is an indication that the patient does not have a need for the ICD.

54. The method of determining the need for an ICD of claim 51, wherein the SNP is bi-allelic.

55. The method of determining the need for an ICD of claim 51, wherein the SNP is multi-allelic.

56. The method of determining the need for an ICD of claim 51, wherein the SNP is selected from the group of rs10505726, rs2716727, rs564275, rs7241111 and rs3775296.

57. The method of determining the need for an ICD of claim 51, wherein the SNP is selected from the group of rs1439098, rs12666315 and rs6974082.

58. The method of determining the need for an ICD of claim 51, wherein the SNP is selected from the group of rs4878412, rs2839372, rs10505726, rs10919336, rs6828580, rs16952330, rs2060117, rs9983892, rs1500325, rs1679414, rs486427, rs6480311, rs11610690, rs10823151, rs1346964, rs6790359, rs7591633, rs10487115, rs2240887, rs1439098, rs248670, rs4691391, rs2270801, rs12891099, and rs17694397.

59. The method of determining the need for an ICD of claim 56, wherein a patient having a TT genotype for rs10505726 and a TC or a TT genotype for rs2716727 does not indicate a need for the ICD.

60. The method of determining the need for an ICD of claim 56, wherein a patient having a TT genotype for rs10505726 and a CC genotype for rs2716727 indicates a need for the ICD.

61. The method of determining the need for an ICD of claim 56, wherein a patient having a CC or TC genotype for rs10505726 and a TC or a TT genotype for rs564275 and a GG genotype for rs3775296 does not indicate a need for the ICD.

62. The method of determining the need for an ICD of claim 56, wherein a patient having a CC or TC genotype for rs10505726 and a TC or a TT genotype for rs564275 and a TG and a TT genotype for rs3775296 indicates a need for the ICD.

63. The method of determining the need for an ICD of claim 56, wherein a patient having a CC or TC genotype for rs10505726 and a CC genotype for rs564275 indicates a need for the ICD.

64. The method of determining the need for an ICD of claim 51, further comprising the step of testing for indicators selected from the group consisting of a screen for Coronary Arterial Disease (CAD), Echocardiogram, Ejection Fraction (EF), and electrocardiogram (ECG) analysis.

65. The method of determining the need for an ICD of claim 51, further comprising the step of testing for genetic susceptibility to SCA.

66. A method of detecting Sudden Cardiac Arrest (SCA)-associated polymorphisms comprising the steps of extracting genetic material from a biological sample and screening said genetic material for at least one Single Nucleotide Polymorphism (SNP) at position 51 in any of SEQ ID NO.'s 1-822.

67. The method of detecting SCA of claim 66, wherein the genetic material is combined with one or more polynucleotide probes capable of hybridizing selectively to a SNP at position 51 in any of SEQ ID NO.'s 1-822.

68. The method of detecting SCA of claim 67, further comprising the step of determining an allele at position 51.

69. The method of detecting SCA of claim 67, wherein the probes are oligonucleotides capable of priming polynucleotide synthesis in a polymerase chain reaction.

70. The method of detecting SCA of claim 66, wherein the genetic material comprises DNA.

71. The method of detecting SCA of claim 66, wherein the genetic material comprises RNA.

72. The method of detecting SCA of claim 66, wherein the genetic material is amplified.


SEQUENCE LISTING (822 sequences)

SEQ ID NO: 1 - DNA, 101 bp, Homo sapiens
tgtacctggaactgaaggatggcagatgacaagccaggcagggaggaatgracctggattcctggtgaaggacgtggatatatcttgtggggtataacttg

SEQ ID NO: 2 - DNA, 101 bp, Homo sapiens
ttatttttaagtaaaaaaaaaaaaaagaggaacagaggttatattttttgytatactcaatgagctatctttggataccaataccctatactattcactcc

SEQ ID NO: 3 - DNA, 101 bp, Homo sapiens
gatgttttagtttgctccatgaggaaatagtttccctttttctatttggcrtataaattttcttgctgattataattcttatgtacattacatttttatta

SEQ ID NO: 4 - DNA, 101 bp, Homo sapiens
atagtggtcactggtgacgccaataagagcaacaaaattggaacttttctragcacacaagtgccattgtattaaagagaatatttgctttggatgacaga

SEQ ID NO: 5 - DNA, 101 bp, Homo sapiens
ttatataaactattttgattatgcttcttatatctaaggcagcctgtatarttcatttttttccaaagttagatcctagctgagtagtgaggtatgtacgg

SEQ ID NO: 6 - DNA, 101 bp, Homo sapiens
aaacaatacataacatttcacctttttctaaaacctccaacttttgtctgmtctttgaaaataccaaagtctacctagtctgtgagtacaccccaaattgt

SEQ ID NO: 7 - DNA, 101 bp, Homo sapiens
atgctgccttactctttaagaagatactcatcttcaatagaagaaactgtrtctgtggaacagtaaaaaggccagacactgttcaagatatgtaacacaaa

SEQ ID NO: 8 - DNA, 101 bp, Homo sapiens
atgctgttctctttgagatcctaaattgtttatgagcttggggagctagtyatataatgatgttattttctaaaatgtctgtgtgaacatctttttttttc

SEQ ID NO: 9 - DNA, 101 bp, Homo sapiens
tgattatttttggaaaatgaagtcacatacatatagttctgcatcttgctyttcttacttttcaatatcctgtggaaccctctccaagtcaactggtatag

SEQ ID NO: 10 - DNA, 101 bp, Homo sapiens
atattcaaaataaaaaatttgtaataatcaaagaatatcttctcccatatkagattaatttggcccccaatcaaccacaccacagcctagtaaggtcttaa

SEQ ID NO: 11 - DNA, 101 bp, Homo sapiens
aaaaaaaaactgcacaggcttgaaaactacatttactttacacagctagaygagagaacttgtcatgtcttctaagaacaatgacaatgatacaaactaga

SEQ ID NO: 12 - DNA, 101 bp, Homo sapiens
tacatgtgaggcagacctgaaagacataaatgccagtttccctcttccccmtttggagtgtgtgcttttgatttccctggaggctacatttcctgatctgc

SEQ ID NO: 13 - DNA, 101 bp, Homo sapiens
tctgaagtcagaggttttggatatgaggcagtgctctgacattcactggaytctgaggctcatatgtgtcccagttgagtaatcacaggttccttaaccct

SEQ ID NO: 14 - DNA, 101 bp, Homo sapiens
ctttatggtatggggaaaaggaggatgatgacagcctctgctgcagtgtayaccatccactatggttgctgttcactctcacctccatggagcatcctctg

SEQ ID NO: 15 - DNA, 101 bp, Homo sapiens
gatccaaaagccatgtttgctgtctcagtggggtagatcacggcctagggyatggtctgagtggggcactgcagaagtgagactgacctcaacaactgcat

SEQ ID NO: 16 - DNA, 101 bp, Homo sapiens
tcttattgatcctattttatcagtctcctctactagaatgtgacctccacrtgaatcgggattttttttggtctatttactggctttactttctacattaa

SEQ ID NO: 17 - DNA, 101 bp, Homo sapiens
caagactccgtctcaagcaaacaaacaaaagagcagtgacagcttggttayggttctgtgaattgaaatgctaggcttcccttagggttagttcctccata

SEQ ID NO: 18 - DNA, 101 bp, Homo sapiens
ttttattatcaggccttggcagccacacttcaactttttacaggtaactcrctggaccctcacagtgactcttcaaatgaggttttgcactagacccattt

SEQ ID NO: 19 - DNA, 101 bp, Homo sapiens
tgctggtggaactcactggtcaatattccttttacccatatatagacatcytgtgtcagtgaacttcaaagctgctgattagttttttcctccataatatt

SEQ ID NO: 20 - DNA, 101 bp, Homo sapiens
atgtcaagataagctgattatcctagaatatccaagtgggtccatgatacygagaagcaggaaaatggtaatggaacaatcagtccagacaagccatgcaa

SEQ ID NO: 21 - DNA, 101 bp, Homo sapiens
tgctcaaatctgctctccatacccactagaaaatcctaaaagaaatatagycttaaatacagtttttaggctccatcaccttacctatcctggctgttgtg

SEQ ID NO: 22 - DNA, 101 bp, Homo sapiens
ttccccaggggaaaagtggactgcagaaagacactcactcaccctctctcrtagtggggattcactctcagttcctggtctatcatggtcatataagctgc

SEQ ID NO: 23 - DNA, 101 bp, Homo sapiens
gggcctgatgtatcacaagggtccttataagaaagaagtggggattgaaakatgttatgctttgcagatggaggaaggggccacaaaccaagaaatgccag

SEQ ID NO: 24 - DNA, 101 bp, Homo sapiens
gttgttggtgtaatgaacgtatttaaccttttcctgatagtcaagttcttyctcaatttaggcatcaatctcatctgtgctgtctatggtgattgccttca

SEQ ID NO: 25 - DNA, 101 bp, Homo sapiens
cgcagtgcttctgagagcgggaatccgcgaactggagtcccgtcttccttytggcgtcctgtcttccttttggagctccccctcaaggaccccgggagccc

SEQ ID NO: 26 - DNA, 101 bp, Homo sapiens
aaatgacaggaacacatggacacatagagggaaaaaatagatgctgggacrtacctgaaggtgcaggatgggagaagggtgaggactgagaaactagaaaa

SEQ ID NO: 27 - DNA, 101 bp, Homo sapiens
aaagaagattctcccttttgaaaataatggaactccaggaaagccaaatakgttcaacataattatgagaaagaagtgtgccactgtcagattggcattta

SEQ ID NO: 28 - DNA, 101 bp, Homo sapiens
ggtagatcacaattccatgaagagcaagcaaatatgaatggagttggatgmtaaacagcaaagtgatatttaagtgatcagactacatcacactttttttc

SEQ ID NO: 29 - DNA, 101 bp, Homo sapiens
ccagttgtctcactttttttttttttaccacgtctgtgttcctcatctcayagcaacctggctttaacttccatcccttcacaaaaattacagaagccacc

SEQ ID NO: 30 - DNA, 101 bp, Homo sapiens
ccaagttgtacactttaaatacaatttttgggttaatctaattcctttgcygtttcatgtaaatttttgaattagattgtttacatctataaaaaataagc

SEQ ID NO: 31 - DNA, 101 bp, Homo sapiens
tgcatacagacctaaaatatcgtagttttgaaatgtgcattgagggaaagmtaaggattagcctggtggcataaaatatgggcagcagctggaggtgaagt

SEQ ID NO: 32 - DNA, 101 bp, Homo sapiens
cctctgctcattggcatgccccctgacatctgtttcccctgtctttcactrttggaagtctcagagcctagaaacaattggacacagacatttccaattct

SEQ ID NO: 33 - DNA, 101 bp, Homo sapiens
actgtctctacaaggaaaactataaaacaacaatgaaagttactgaagagracattaaataatagaaagttattccatgctcatgctttgaaaaaattatt

SEQ ID NO: 34 - DNA, 101 bp, Homo sapiens
caggaagcactggaagtagctagcaagaataatagttccttgaggatgggrccgatgctatgcttttttatgatgctccactgaacttacaataattctgt

SEQ ID NO: 35 - DNA, 101 bp, Homo sapiens
ttactttctgggctgatgaaagtgttctggaatcagcagtgatggttgtgyaatcctataagtacataaaccactactttttaaaaagctttgtaaataca

SEQ ID NO: 36 - DNA, 101 bp, Homo sapiens
acctcagttttaccatctttaaattgggtgtaataatgagatctatcttayagctctgtgaagattaaaggagttgaaattggaaatggtaggtgctcaac

SEQ ID NO: 37 - DNA, 101 bp, Homo sapiens
gtttctacattctgaccttgttctgtgctctgcggggctgatctcaatggmaagtgtctctggatttcccttatcccttattggctttgactaactggggg

SEQ ID NO: 38 - DNA, 101 bp, Homo sapiens
tgggttgacatatgagaacatggaagggccatgtaacaggtttagtctagkcagccaagccttttcaaagtgatttctaaattgggtaatggagggtgggt

SEQ ID NO: 39 - DNA, 101 bp, Homo sapiens
aagaaaacaaaaacagactctttcttacagagtaagaggaaaaacagaaartgaaggcaaaaaacaaatgaaaagatgccctttctattttctgaagccag

SEQ ID NO: 40 - DNA, 101 bp, Homo sapiens
aaggcagaacgccagaacaggtggatatgagtcccaagccactgtgctcamtcaccgtgctaattctgcctccctgcagctgctgtggctgataaggagga

SEQ ID NO: 41 - DNA, 101 bp, Homo sapiens
ctaccaacctgccttccttcctgttaacttaatgagctgttagtgctcaayctaatggtgagttcattgtccttatcttatttgacccagcaacagcttgt

SEQ ID NO: 42 - DNA, 101 bp, Homo sapiens
gttgacattcctggtaggaagatccacaaagcatttggtcctattgccagrggtatcatcactggttctgaggagtggaaagagtacacaggcaaggcaga

SEQ ID NO: 43 - DNA, 101 bp, Homo sapiens
caacatcatgtcatttctgtgagcagagccaaacattgttgcctgagagarccccaaggagggcttgaaaagagtttctcatcagcaatctcatactcatt

SEQ ID NO: 44 - DNA, 101 bp, Homo sapiens
ttccccactagagtggaagcatcctgaggacagggacctttgctgctttgytcaccactcaatcatcttgcccagaactgagcttggtacattgtaagatg

SEQ ID NO: 45 - DNA, 101 bp, Homo sapiens
tgactgtatatacaaaggtggaattgctggatcctatgtgttaaacctttracctcttgaagaattggcagactgtttgccaaagtagctgcactaataaa

SEQ ID NO: 46 - DNA, 101 bp, Homo sapiens
tctttttggctattccccttctgtgccctttttgcagaagtaaactctgtkgggaagggtaaatgtgtagcctcaaattcctcattaaggttttattttat

SEQ ID NO: 47 - DNA, 101 bp, Homo sapiens
gctcagtaaatatttgttaaatgaatgaagtgtatgtttctgcaaatgctrtcagaatctcattttatctctctgacaagactgcacctttagtgcaggga

SEQ ID NO: 48 - DNA, 101 bp, Homo sapiens
agagaacagcattgacagagacgattagtttcctcccccgcccccagtccrttggcctctgttgctaataacgcttggttgaggattatattaaaatgagt

SEQ ID NO: 49 - DNA, 101 bp, Homo sapiens
atgcttccagctctgttatttttcttaaaattgcttgggccattcgagctyttttttttgtttaatatgaattttagggtttgagtacattgaagcttttt

SEQ ID NO: 50 - DNA, 101 bp, Homo sapiens
acaaagtttgtgtataatacatgccaagagggtaggaataaaataccattygctgtcaagatatatttctaaacaagtttattaggaaggcagtagcagat

SEQ ID NO: 51 - DNA, 101 bp, Homo sapiens
agtgtttattaatgaactagccatagtaaaattacagcccatttaaacatycctctttgactaacactagtgtctatcccttgccattgcagcaatgatct

SEQ ID NO: 52 - DNA, 101 bp, Homo sapiens
gaggaaaacaatttctcaatccacggttatttctttgttatactaagaacmgtgcccaatacttatggaacaaaataagcctatcatttggacgtctccta

SEQ ID NO: 53 - DNA, 101 bp, Homo sapiens
agaggcaagtgtcagaaattaagcaagtaaacaacagaacactgtgagccrttggtttgtaacatgacagctgcctgtctgtgcctcttactgtgtctgtg

SEQ ID NO: 54 - DNA, 101 bp, Homo sapiens
gagctaggctaaaatcaggacccaagaacctcacctaagatattttacagrgataaaaccattatctattcatttttcaaaatccccctttaatccaaatt

SEQ ID NO: 55 - DNA, 101 bp, Homo sapiens
cctttttcctctctctagaaagggaggatcaccaggaagaaataagtccarattccccatcagttcagtggtatggagtccagagtcagaatataattttt

SEQ ID NO: 56 - DNA, 101 bp, Homo sapiens
cttatatgagctatgaattagcccgaccaccatcactgctactgctactaygccccagactctctgtgctgctgccttgccagcctgctgtgccctgctga

SEQ ID NO: 57 - DNA, 101 bp, Homo sapiens
ggtgtttggcagtgctgttgttcaaaaatatggccaaggcttcttaaatayactgactgttggattcccttccctgcctccactccctcatctgctgaatc

SEQ ID NO: 58 - DNA, 101 bp, Homo sapiens
cttgactaagtggagggtattgtggagtagagcccttctgaataacagcarctaacattctcatagcactaactgcacccctttgaggtaggcggtcttat

SEQ ID NO: 59 - DNA, 101 bp, Homo sapiens
gcaacagagaaaaaaatgttttttgtttattttagcatgtttatttttggyccaagcctttatcaggttggagttggaggctggggaggaagaataacaaa

SEQ ID NO: 60 - DNA, 101 bp, Homo sapiens
ttttaaaaatacaaattaaaaattatctattggacagagccatgtgtagaycttagcctttgcacttgcaaatcaaagctttacaagagatgctctccaaa

SEQ ID NO: 61 - DNA, 101 bp, Homo sapiens
ttaaaaaaacttcatttacaccagaatgatttccgtctgtcactcattgaytttacctctttttttctacctctaattactataaaaatatttgggatggt

SEQ ID NO: 62 - DNA, 101 bp, Homo sapiens
ggcaaaggggttaggtgtcaatgcctggctgatttcctgcattacaaaatktacctcttacttttctgtcttcctgatgttaccccctcttttctttcacc

SEQ ID NO: 63 - DNA, 101 bp, Homo sapiens
tttccctgataaaaaggcatcttgtccacagctgtacttgttttcttattragtgatcctggttatagaacatgtgacttcaggcataaaattctttctac

SEQ ID NO: 64 - DNA, 101 bp, Homo sapiens
aggaaacacaaacttctagaacttttaaattgttaaacatctttgtggaaktaactaccattttcaccaaatctgcaaatcatattccaacaagttgtaaa

SEQ ID NO: 65 - DNA, 101 bp, Homo sapiens
tgtggctgttaagtggtgactgaagtagaatggaggtgaaaataattcaaratggaaagctaaaacaaccgagaggcttggaagctgaagaattccttcat

SEQ ID NO: 66 - DNA, 101 bp, Homo sapiens
cacatacgcatatcctcctcaattttataaagaaatagaagcaccattccrcaccttcatattccacccttaatcattgttaagttggttgcatgtcttcc

SEQ ID NO: 67 - DNA, 101 bp, Homo sapiens
gcaaagagggccagtagttacactgcaccattgtggtgacatcaccctatrtatgtattttttaaataacttgttaatgcatatttccctagctagactaa

SEQ ID NO: 68 - DNA, 101 bp, Homo sapiens
ttttggctgttaggctgtagagactttatgagggtgccaaacttggaagamatattgaaggtagactcaacagaattttcacaatatgaaccctgtgagac

SEQ ID NO: 69 - DNA, 101 bp, Homo sapiens
ctattgtgaggcagggtgtggaaatcgtgattgagatgacaaggcacccarttgtactcatataaagaacactgcttgcgcgtatgattgctgttcaggtc

SEQ ID NO: 70 - DNA, 101 bp, Homo sapiens
tagtatgcttattaaatctgcagatgaatgcatcttgtcaaggaaaatttyctatgttacaactgaatttcttctatttcacatgttgaggtctctttgga

SEQ ID NO: 71 - DNA, 101 bp, Homo sapiens
gacaggtcttctttcctgccagagggagctctgaagacaactagagaattytgggcctgaaatttcaatctagttagaaagaaaaatgaggcaatgatttt

SEQ ID NO: 72 - DNA, 101 bp, Homo sapiens
gacagggcacgtaggaatatggaagtcagaaggacaacacagctctgctaygtcccggttcttggtaactttcttaaccccactatgctttatctttagtt

SEQ ID NO: 73 - DNA, 101 bp, Homo sapiens
tgaggagagttcctgggccaagggctggctggcccatgtgacttttggggkctcaggaggagcctgttgtgttggggagtctctctgctcaggtcctgtgt

SEQ ID NO: 74 - DNA, 101 bp, Homo sapiens
gccccttggctggttcttacccatcagcaagctctgaatgcggtcgtaatrtgtgaagttgtaggtgctgctcgtggaggctgcctcatccctgggcagcg

SEQ ID NO: 75 - DNA, 101 bp, Homo sapiens
tgggcaaattcgctatgcatcaggctgacggcctggaggaagcggcgatcmtgcggggtggccacctgcggcaggtttgcttccagaagaggacacagagt

SEQ ID NO: 76 - DNA, 101 bp, Homo sapiens
gggttcccacccagacagacggactcaagaactcacgcactgcctctgcaycctctgctgccaatgaaaatttaaatgagggcaacaggagatcagagatg

SEQ ID NO: 77 - DNA, 101 bp, Homo sapiens
tgaaatctacaaggtgcctttcatcacgagagctgagcgatgacccctgartgaggagggccaggagcttagtcccatctcagagacagacactgactcag

SEQ ID NO: 78 - DNA, 101 bp, Homo sapiens
tccttgaccccattcgccctcttacaaataatgaggttcagaaggcaggtrcaccagatgggagggagaaacaaaaataaagataaacgaaacaacattta

SEQ ID NO: 79 - DNA, 101 bp, Homo sapiens
gcacttcatttattcaccaaatacctgctttggaaaataattggagtcggrgggagcagcaagaagggtgaaatagggcagtgcagggctcctggattggg

SEQ ID NO: 80 - DNA, 101 bp, Homo sapiens
ttcataggcatgcaagccttcttatgaactaactgcacgtgccagggatcraggttgcacactccttataagaatctaatgcctgatgatctgaggtggga

SEQ ID NO: 81 - DNA, 101 bp, Homo sapiens
atcatggcagaaggcaaaggagaagcaggaaccttcttcataagggggcargacaatgtgagtgccagcagggaaaatgccagattcttataaagccatca

SEQ ID NO: 82 - DNA, 101 bp, Homo sapiens
gctgaactggccatggaaatggcagcctgggcaacaggttcatgaaaacaracttttcacacctggtcctgctctccaggcctgagcgaactccatgtgtt

SEQ ID NO: 83 - DNA, 101 bp, Homo sapiens
ggctcttgtgggacagggctagtggaacctacttgggtgtctccattgcgrgcagaacgtaatagctgtgtgtagaaggtcccactggatgaagggccagt

SEQ ID NO: 84 - DNA, 101 bp, Homo sapiens
tggctggaggaacccaggaacaccctgagcatccatgttcttaatgacaaragagggaacacagatttggcttccctttcttcataagaaaagaaagaaaa

SEQ ID NO: 85 - DNA, 101 bp, Homo sapiens
catgcatatccagaaactacagtaatttacaggggcaaactctgcaactargaaaaggagacagaactgtttccactcaatgcattcctccatcaaagaac

SEQ ID NO: 86 - DNA, 101 bp, Homo sapiens
ttgtgtttctgtgtggctgaaatcgtgtcgtaaagttagaagaaaggctgytgtggggcctgcgttgcttggcagaatgttccttaccttttgatttgcag

SEQ ID NO: 87 - DNA, 101 bp, Homo sapiens
gtgccaagcagagcaggtagttggctaagtttgcctccaggaaagaagtcyctggagagcgagctggttctagaaagctccattattatattcctattgct

SEQ ID NO: 88 - DNA, 101 bp, Homo sapiens
gtcagtggtgatattctctttatcattttcattgtgtccatttgattcttytcacttttctttgtctagctagcagtctatctattttattaatttttttc

SEQ ID NO: 89 - DNA, 101 bp, Homo sapiens
cccatgtaagacacccatgaaacaatgctctggtcataattagtctctaamctttcaaaatgcctgcttcagtgacctcacctgctattgaacacgatgcc

SEQ ID NO: 90 - DNA, 101 bp, Homo sapiens
agccacctctcatttgcatggtggacagctgcggctgacaggcaaacaaaratgtctgcggccatggcagctcctagagaaactcttctctccttactctc

SEQ ID NO: 91 - DNA, 101 bp, Homo sapiens
ctgcgcttcccccagaaagcatgcctgggtgaggggccaggtgacacttcytacgatctggattttaaaatatgtttgcttatgccttcaccctccaccaa

SEQ ID NO: 92 - DNA, 101 bp, Homo sapiens
gcgctcacgggagggcggatgtggagagggcagaggagcaatggtgacctrggaaggtaccctgagcggctacgctaggatctctgttctgcagacttctg

SEQ ID NO: 93 - DNA, 101 bp, Homo sapiens
agggaagcatcagatgtcactggcttgggaaagatattccagaaggaaggracaggttgtacaaagtaaggtaattttgtttggggaagctccagcaggtc

SEQ ID NO: 94 - DNA, 101 bp, Homo sapiens
agttatcagcttattgctattaaaaataacactaaacttttgtttatctamagagtgtcaggtaagcaagtgaacattttgatgcaaaaagaaatcacttt

SEQ ID NO: 95 - DNA, 101 bp, Homo sapiens
ggctgagtaaattaaggtacatctgtattaaggaataaaatgcaactacgraaaatgataaactagatggaggggtgcctatgacactgtaaagtttaaca

SEQ ID NO: 96 - DNA, 101 bp, Homo sapiens
tggctgtgttctgagtgggagtgtcctaagagtgagagttcctagtgaccyaggcagaagttgggttgacacttcttgcaagatttctgatgacctagcct

SEQ ID NO: 97 - DNA, 101 bp, Homo sapiens
ggtctctgtggattcccaaaggaggtttcaaatggagtcattgtaaagacrattcatgatcttagaagtgtctcatgcagtttcctcgtgatggtcttgtt

SEQ ID NO: 98 - DNA, 101 bp, Homo sapiens
caggaatcccaattatggggaaagaagatgagcttctgagactattccgakccacaagatttttcaaattcttcacaatctctgtctcatggatcagagag

SEQ ID NO: 99 - DNA, 101 bp, Homo sapiens
cactgtaccttcgcagcacgaggagaggagagttcgaaaccacaaagctcyttcctttctttcaggagaaagaaaatggaggatgggaacgtcatcagccc

SEQ ID NO: 100 - DNA, 101 bp, Homo sapiens
gggcctcaattttctcagctataatatgggctgacaagagtaaacgacaakagcaaatgagttaatatgtgttgcccctgatgttacagtggataacgatg

SEQ ID NO: 101 - DNA, 101 bp, Homo sapiens
aatcttaaacagtaaagtttcacgaagacaaaaatctttttgatcaatcaygtctcttttacaaagtttacaaggaaagtattcatccctaaaactatttt

SEQ ID NO: 102 - DNA, 101 bp, Homo sapiens
gagttacttatacaaaattacacactaagagatttgtatgtataattgtgkgtacacattcctagtattttcctgatataaaaaaattattcctatataag

SEQ ID NO: 103 - DNA, 101 bp, Homo sapiens
gaaggagtttggatatattccctcttctttaatttttttgaagaatttgartagaattagtgttagttctttacatgtttgttagaattcagctgtgaagc

SEQ ID NO: 104 - DNA, 101 bp, Homo sapiens
agttagtacaggagcggggccaggagagtgctgtcccctcagctccagtgrgtggctgcccatccagagcaagcctgcagcccccacccgcctcctccttt

SEQ ID NO: 105 - DNA, 101 bp, Homo sapiens
tcttgaatgcaggaactattatataaaagcattgcagctcttggtggttgyggcagagacgcagagaaagccagtttgcattgaaggaagggtacagcaga

SEQ ID NO: 106 - DNA, 101 bp, Homo sapiens
tgctatagtacacatagcaaatctgcaaaagtgctagctatcattattatmtgaggcttttgacccagctctcagagaagctggaaatttgcatttttatg

SEQ ID NO: 107 - DNA, 101 bp, Homo sapiens
ggagaatgcataatgaggctgaatgagaattagatgcttaattgaggcctrgaaaagggaaagaaaaagccagacatgtggaatgtgatcagaatgcagct

SEQ ID NO: 108 - DNA, 101 bp, Homo sapiens
acagactgtccttggaatgttggaaagttatttggaaagtccttatgagcytggggcacattcttctgaagagctttcttgattaggaaaatcctgtgctt

SEQ ID NO: 109 - DNA, 101 bp, Homo sapiens
tacacacaaattcatgcccacacccatagacacacatatacatatatacaygcatgtatatgtccgtatagagagctctatgctggaatatacaaaaacat

SEQ ID NO: 110 - DNA, 101 bp, Homo sapiens
gagcttcaggacttcaagtagatcacaaaaaaagtgtggaatttccatttyggtgcagaaggacagcctcaaaacagtcaaggtctcgagcagggaaccca

SEQ ID NO: 111 - DNA, 101 bp, Homo sapiens
gcctggggggtggtaatttgggagccactgaaatgaacttgcaaaaggttktgggactattcatttatctgcagaaggctcagaaatttcattagattctc

SEQ ID NO: 112 - DNA, 101 bp, Homo sapiens
tttgtttttttgtattttcacaataaatatgaaaacagttttaatttaatkattatgaacaaaaaaggatgaaaaccaatagtcagtttctttgtaaaatt

SEQ ID NO: 113 - DNA, 101 bp, Homo sapiens
caccacacaggaagggattttgtctgtcatgttcactgctgtgtccccagyatgctaagtaggggccagggtcaaagtaaatgcttgatgaatctttgccg

SEQ ID NO: 114 - DNA, 101 bp, Homo sapiens
tccccacttcttgcataaagggtagcattcatgagcataccgttctgcacyttgcttttttcatttgtgtcttgaaacctgttccctgttggctaagagag

SEQ ID NO: 115 - DNA, 101 bp, Homo sapiens
gccttggacctgctgggcccagccactggctgtctactggacgatgggctygagggcctgtttgaggatattgacacctctatgtatgacaatgaactttg

SEQ ID NO: 116 - DNA, 101 bp, Homo sapiens
ggccctcatgctgtaaagaagttgagttctggaaactccaagttatcatcrtccaagtttagcaatcccatcagcagcagtaagaggaatgtctccctcct

SEQ ID NO: 117 - DNA, 101 bp, Homo sapiens
aagagtgcataggagttttctaggcagagaaaacaaccctgcaggcgcacrttggctcccattcctggattgagggcgtggccatgaagtctgggtgctgc

SEQ ID NO: 118 - DNA, 101 bp, Homo sapiens
caggaggggtcaacttggagggccaagcaaccaggggtcacatgggcatayggctgagcctggacccatccacctgactactatgctattatagggctccc

SEQ ID NO: 119 - DNA, 101 bp, Homo sapiens
agaagtttctttattgagaatgatattcattagtaggcattcaatgataargacacagcctgattttaaagatttcctttttttttttttttttgcacatg

SEQ ID NO: 120 - DNA, 101 bp, Homo sapiens
ctccaagggcggatggcctgaccgggataagacccgtgaacagatagtaartgtgggtttggcatttggcaggaaatgcttgtggaattcaggaggcaact

SEQ ID NO: 121 - DNA, 101 bp, Homo sapiens
tgtgctcaggcaagattatggagcgagcttggttttgtcctactccatcgyggtcagagtggccccatctgatatgagcgttctgtgagttttttttatta

SEQ ID NO: 122 - DNA, 101 bp, Homo sapiens
gattacaagcgtgagccaccacacctggccttgaggtcacctttgcatgcraaggctgtatactgctaacacctgtgacatctcctgtctgatggtgtcct

SEQ ID NO: 123 - DNA, 101 bp, Homo sapiens
aaatttttcctgtaattgaccaagtagcaaatatattcagctttgctggcygtaaatttcctggcaatgactcagtcctgccgcggcagtgtggttaacag

SEQ ID NO: 124 - DNA, 101 bp, Homo sapiens
tgtcgaaaaacctatcaacaattccttagtttcaccacttcaaaaaatttrttctagtgtcaaatcccacattttaaataaatacagaaatgattttgatg

SEQ ID NO: 125 - DNA, 101 bp, Homo sapiens
gaaggagggatttggagccagggcagacagagcagcatggtgctgggagarcaagaggggcagccagtgataaggagagcacagggagaaccacagcctgg

SEQ ID NO: 126 - DNA, 101 bp, Homo sapiens
gcacattatctatgctgtttgttataggtaatagtttcagcaaactagacmggaaggaaaaaatgcattaagagtgaaggtgaaagagagagcgagagtgt

SEQ ID NO: 127 - DNA, 101 bp, Homo sapiens
acaagatattccctctgatctctggccctctcctccagccctctccaagarggacattgtccttgcctcctatcccagagagctggcaaatattcccctac

SEQ ID NO: 128 - DNA, 101 bp, Homo sapiens
gatttctcctgtgtgggcaagtcacacacaaaactccagaaatacatattyaaaatgctcctagcttccctctgcattagtcacaataacactaaatgctg

SEQ ID NO: 129 - DNA, 101 bp, Homo sapiens
agcaagactccatctcaaaaacaaaaaaggcaaattaaatttatactaacrtcagcaaactagagaatttaatggctcatgtaactacaggtagagatggg

SEQ ID NO: 130 - DNA, 101 bp, Homo sapiens
atagctcctcttttattactcggtcctggggttaacctcaattgtatccayttactcaactagtgtttaatgagttgccatggtgtgcctcgtacttgtga

SEQ ID NO: 131 - DNA, 101 bp, Homo sapiens
tcatagcttcctttgtacctcaaactaagtagcttcatattcctttgctcrtgcaacccaatcatatttgggaagctgcagatgaaaagcatactgacttt

SEQ ID NO: 132 - DNA, 101 bp, Homo sapiens
gggtcatctgacaataaggccacctaaggtccgccagtagtagttgtagaygaactggtgacttctggcatggtcattagggcaattgttaaaacttttat

SEQ ID NO: 133 - DNA, 101 bp, Homo sapiens
tgtttgctgagccttctctgcgctgtgtatagtactcagggaagcttcacrtaagtgtcttccttcactcatgtgttcgctcaggaaatacgtatttactg

SEQ ID NO: 134 - DNA, 101 bp, Homo sapiens
gccatggacattccgggttcccaagtcaggtggggcccagggataagcatytatttttgatcagcacctcaggtaactcctgtcttcaccatagtttgaaa

SEQ ID NO: 135 - DNA, 101 bp, Homo sapiens
tatcttatttattttcaagtcacaccaaaggaaaggcaaggctcagagaartggattaatttgctggaggctacatagtaagcagagggggtgggatatga

SEQ ID NO: 136 - DNA, 101 bp, Homo sapiens
tataagtgtatatgtagaagaaaatgtccggagtctggagacagaaccaakagagagaattagaggttagatttccagtgcttacacagagccagtgttat

SEQ ID NO: 137 - DNA, 101 bp, Homo sapiens
ctgtacaaagtctgaattttgggggaatctgaagagtctcatttaaatatycagctgattaattataagtgtatatgtagaagaaaatgtccggagtctgg

SEQ ID NO: 138 - DNA, 101 bp, Homo sapiens
tcttctcattacttcagaatacagacatccagtgtttaattctgtttgtgrttatctcataattattaagatatattcataactatttgtttattaatcaa

SEQ ID NO: 139 - DNA, 101 bp, Homo sapiens
agaacaaaagtaggtgattgatatagtttggatatttgtcccctcttaatyttatgttggaatgtggttcccaatgttggacatggagcctggtgggagat

SEQ ID NO: 140 - DNA, 101 bp, Homo sapiens
acaggacatgctcaatgtgggctttttttaaattttttttccttctcttgyttttcttttatttctgtgcgattacctgctcctctgtggtttctttattg

SEQ ID NO: 141 - DNA, 101 bp, Homo sapiens
ctgacaggcagaaatatatgccaccccaaaatatgtcagcctaaaagatgycttctcaattgaaggcaattgagaagaagcagatacaagaaaagctctct

SEQ ID NO: 142 - DNA, 101 bp, Homo sapiens
gaggttgataaacatgatggtgaagatgttgagcagttttccttaaaactrgttctcaattcactgctgatttgtggaaatctggcactgtctataccagg

SEQ ID NO: 143 - DNA, 101 bp, Homo sapiens
tacagtgtctagatgtgctagtgtatccagaatggtgcccaagagagaaamgtaggttaggaatatattgagctgacctattttccatacgtaagtatggg

SEQ ID NO: 144 - DNA, 101 bp, Homo sapiens
aatataaaaacatttgacttaagattttctgaggaagcttaagtagtttcrttgaaggctgaactggtttggtcctgaatctcatcctctatggcataatt

SEQ ID NO: 145 - DNA, 101 bp, Homo sapiens
cccaaactctcctttcgatcctttaatctcccttaatcatctcttgaatcygcctcttcctgtctattctcacacactctgttctaacctagaaccacttt

SEQ ID NO: 146 - DNA, 101 bp, Homo sapiens
gaaaagacctcaaatttgctagtaagattcaacgataaatgcaaaatacayacatctacacacacttacttagaagggtagtaagatagacatatttgaca

SEQ ID NO: 147 - DNA, 101 bp, Homo sapiens
atgcccccgtttaacctctgaaaccttgtcattaaactacagggaattaartccaataataaacccttccattgtcaacagaactctcaatgaactgtacc

SEQ ID NO: 148 - DNA, 101 bp, Homo sapiens
gatgattgtagagcataaagaaactaattcacgtaaaacattttcatgtcyaggatacaggtttcaataaatattagtcagaagcatcgtgatcattttgt

SEQ ID NO: 149 - DNA, 101 bp, Homo sapiens
catcgtcactgggttaggtctcaatgtcggcagggctggctgaggctctcrggaggattatctttccttgcctttttccagcttctagaagccaccttcaa

SEQ ID NO: 150 - DNA, 101 bp, Homo sapiens
actgcccgctctccttgccttcatggggccacaactttctgacttctcccrtttgcttttgcagacacctcctcttcctctagatattcttctccagagag

SEQ ID NO: 151 - DNA, 101 bp, Homo sapiens
ggcaagtccagcaagtctacatatttctagtcacatttccttgcctataayttattaatccatttatcaaatatttattgagcacatacttactatcatgt

SEQ ID NO: 152 - DNA, 101 bp, Homo sapiens
cacaggatggaaacaaaatatcatgagggtccagcagtcttcagagcagtrttttttcagctggggacagaaacaccaggaggcttatgaggagtttctag

SEQ ID NO: 153 - DNA, 101 bp, Homo sapiens
ttgatgtcatttgggacaatggcagaaccgtctccttctccaagttctaamaatgaacttagatgactggcaaaacccccagagtgtgaaggcttgtagct

SEQ ID NO: 154 - DNA, 101 bp, Homo sapiens
catgtgacaggaatatactagatgtatctacaagttttcttatgacacagrtattcatgacatcaatctcatgacacaggtagtaggaatatattttaaag

SEQ ID NO: 155 - DNA, 101 bp, Homo sapiens
aactggaactgctggttaatcttgaatcagacaaagagcaccatggacacytcgaggaagtgcccacagcccagcaacaaaagtttctgcagagatttctt

SEQ ID NO: 156 - DNA, 101 bp, Homo sapiens
aagtcaaactatccgtgtttgcagatgacatgatcctatatctagaaaacyccctaatcttagcccagagcttcttaggctcataaacaacttcagcaaag

SEQ ID NO: 157 - DNA, 101 bp, Homo sapiens
ggtggcattatttaaaatgtactaaggtatgactcagtcatcatgctaaarcattattgtaccttatataaacatgactgtaattcgatgttttaaattct

SEQ ID NO: 158 - DNA, 101 bp, Homo sapiens
aaggaaaagtccttctaacttctacagggccaaagcatgcatgtatcataytaatgtcaatcctgtgccagaccctttgtaaaattaagtacttcaaactt

SEQ ID NO: 159 - DNA, 101 bp, Homo sapiens
cctagttggccacagggagggctggtcaactgcaggggcaggcaggggtayacatgacccaggcctagcctggaagtgttctcagcctggtcctgctccgt

SEQ ID NO: 160 - DNA, 101 bp, Homo sapiens
cattttctacaattgtgaaaatcagacaccgcagtaggattagtgtaagcrtcgtggtttctaggtagtcttctctgacacctaggcagaatcagggccct

SEQ ID NO: 161 - DNA, 101 bp, Homo sapiens
gccttcaaagcggcagtggccacccacacagggaactagtgtttgtgagargagaatgaacgttgtttgtaatatgttggtgtgaattgtcagcagagcac

SEQ ID NO: 162 - DNA, 101 bp, Homo sapiens
gctgaaaggtttccatgtggaagcccctgactaccaccaaccagttcaggygagagacctgaatcctttcccccttttctttttaccttttctgaatccta

SEQ ID NO: 163 - DNA, 101 bp, Homo sapiens
atctcaatatatttcaacaatgggaacttctgcggggcacaactcatgtcyacagcctcgtctatgtacagagcccaaagcagcaccactatcagtttggg

SEQ ID NO: 164 - DNA, 101 bp, Homo sapiens
ttctaccaccgtagatccgttttgccttttgtgtctggtttcaatgcatcrtaggtccacgacatccttccacaggtaccggccactcattcctttccttg

SEQ ID NO: 165 - DNA, 101 bp, Homo sapiens
ataggcacatatcggatctcccagcctggtgactcttccgtggtctaatckgaacacctctggcctgccacacctctggccagcctccagttagctgcttt

SEQ ID NO: 166 - DNA, 101 bp, Homo sapiens
tcctagggaacgccctcttctcgctgcggccctggcgtgtgtcgctggatkgtgagggccccactgcattggtctccatgtgctctgccttctcaatgtcc

SEQ ID NO: 167 - DNA, 101 bp, Homo sapiens
agatgggggcagtcctttggcaggggtgctcaagttggtcgattatcccarcggtgccagagcggcagtgatttgtgggtgggcaggctccttccctaggg

SEQ ID NO: 168 - DNA, 101 bp, Homo sapiens
tctgctgcagttcatagggttcttcctgttggtctccataccactcacccraagcatgcgagaagctgcaggggcttgggggcagttggagttcatgtggg

SEQ ID NO: 169 - DNA, 101 bp, Homo sapiens
gatgtatgtgtataaattgcactcatggctctaaaacaaatcagcagaacmcattctagaaaaaatcgcattcaagagatactatactaatagattatgta

SEQ ID NO: 170 - DNA, 101 bp, Homo sapiens
aaaattactcctggcctcagctgcctcatgtctgggtccctccctgccaayagatttgtgatggatatttacacgctggaagtgactgggccatggtctca

SEQ ID NO: 171 - DNA, 101 bp, Homo sapiens
gggagaactacagttcccagaagagtgtgcggaagaagcggcccatgctcycggaagacgctgtggttgagcatcatgggagttgtagtactcctgctgct

SEQ ID NO: 172 - DNA, 101 bp, Homo sapiens
ggccatccgtggggcctgcaggagaacaagtggaatctgcagcatgggacrtctctgcctagagcctgtgcaaacaatggcactgtcctcatcattgaggg

SEQ ID NO: 173 - DNA, 101 bp, Homo sapiens
aaacacaaggaggcaccgaggctgctgtacaagagttggttcctgctcacyccacaaactctacttccacctactgcaaaaggttctgtccttttttttaa

SEQ ID NO: 174 - DNA, 101 bp, Homo sapiens
tgctgaccagggaatacctccccattgaagcctaggccagattccagtccrttttgaccataccccatcatggtattttagagtacacctgaataagatac

SEQ ID NO: 175 - DNA, 101 bp, Homo sapiens
cacgcccccacccgccgcagcccctactcactcttcgtataggagagccaytatgtaggtgagggccaccagcaccgtcaggagcaggcccgtggggctgg

SEQ ID NO: 176 - DNA, 101 bp, Homo sapiens
cagtccccacatttgcattgtccccaaatctaacccaagctgaaagacatyaggcctatcttcttgctttatgcataatggcagatctccagggagggaga

SEQ ID NO: 177 - DNA, 101 bp, Homo sapiens
gccttttcattcccctctttttttaataaaggaaagccaattttaccgggrgtggcaaagtgtctggagaaaacataacatttcttagtttcctttgtagc

SEQ ID NO: 178 - DNA, 101 bp, Homo sapiens
tgtgtgcgttttcctgagtgtgcaggagtacgtgataatttcctgctaggrtggaatgacttccgggtccatgagtgtggaattagggtcagctctgggtt

SEQ ID NO: 179 - DNA, 101 bp, Homo sapiens
cagtttctgaggcccggttctcccccaggggctgggctgcaatcagcaggkactaaatctcactgccaagggcctgggccaaggcatccaactctctgtgc

SEQ ID NO: 180 - DNA, 101 bp, Homo sapiens
ctgaacagcaaacccagaggccattgcagctgcctcggtattctacacccyccttgggtctggaagttgttggaggcaggcataccagactgtttataata

SEQ ID NO: 181 - DNA, 101 bp, Homo sapiens
gtgctctcatcctaatttagggcccctttctgcctagaactctgtagattyccgccgtctgtgtttttccatcatcccagaccctcagctgcaagctcagg

SEQ ID NO: 182 - DNA, 101 bp, Homo sapiens
cccacttgttctgcagagaaagtgagagggaaaggttgctgatcagatgcygctttaaaatgtaatcataagttttggctcagggagagagagagagagag

SEQ ID NO: 183 - DNA, 101 bp, Homo sapiens
gttctagggcctggaccaggggcttacctaaagcccatggtgcctcctccrtctgaatgggagcctccacagccagtaatgagtatccttcctcaaacctg

SEQ ID NO: 184 - DNA, 101 bp, Homo sapiens
agtagtttcgtctctcagaaccttataaaatggataatagagtagtacccmtccgatagggctgttgtcagggacaaggaactaatacccatgaagcactg

SEQ ID NO: 185 - DNA, 101 bp, Homo sapiens
tcagaaaatatttgcacacacattgtctcttctggcccttgaaacattccytgtgtggctgaagaaagtcaatagtggaaccatttaatagataaggacat

SEQ ID NO: 186 - DNA, 101 bp, Homo sapiens
aaaatcttttagttcctaaaaagcacaaacttaaaaaaaaaagggggaaaygaaagggacttcttcaatttggcaaagaacatctacaaaatacctacaga

SEQ ID NO: 187 - DNA, 101 bp, Homo sapiens
atgttttccatgatgagtgggcaacagttaccacccagggctgctccacaragggaatgaactggagacttcacatgtgttcaatttcttgaaagaaaatg

SEQ ID NO: 188 - DNA, 101 bp, Homo sapiens
acacctggggggtgtactcaccttcttcgatgatgcttttcagcatttctrtgtacatgtccttgttgctgggagctgcgctgttcatcttgaagtggggc

SEQ ID NO: 189 - DNA, 101 bp, Homo sapiens
ttaagagatgatttgagaaagaataaatgttgaatgagcatttattatagrgtcgtttatgctacatttgcattttgactctatttctgccatgcaggatg

SEQ ID NO: 190 - DNA, 101 bp, Homo sapiens
gctcatcagctgtagttagtgtatgtgtactttatgtgtggtccaagtcarttctttcagtgtgtcccagggaaaccaaaagattggacgcccctgtgtct

SEQ ID NO: 191 - DNA, 101 bp, Homo sapiens
acctgcagtggactttgagcaagaaatcagcttttatgtgtcaatccaccrgaatttagggctttctcttaattgcagcaaagcctagcccaccgtgagta

SEQ ID NO: 192 - DNA, 101 bp, Homo sapiens
tcatcctattaaggccaggctgcagaggcgttgcgatggagcagagattgrggagggggtacggtgcgagtctctgcaagatgcacagcaaggcagggagt

SEQ ID NO: 193 - DNA, 101 bp, Homo sapiens
gagtgaggtggaaatgtcggtgcagcctgcagctcacctggttgtcactcrcagatcggcctcggaaagctccaggaagttgatttgggatgagccagcca

SEQ ID NO: 194 - DNA, 101 bp, Homo sapiens
ttttctacaaaactaaacactccaaacacaggcacagcaaactgcatttckaaaggttttgtaagttaaacaagccaaggaagttacatggaaaaaaaaaa

SEQ ID NO: 195 - DNA, 101 bp, Homo sapiens
agtgaaaagttattgtgttcacttgaaagtctaactggcctttagaagggytatgcaactagactcaggcttcaagcatagcaagtggcatcaccaacatt

SEQ ID NO: 196 - DNA, 101 bp, Homo sapiens
acatttgaaacagcatgttaaactgtaagtacatcctcaaaatgcagaaayctccattctcatcaagttacatgctcacagtgacagcctgagaaggtaga

SEQ ID NO: 197 - DNA, 101 bp, Homo sapiens
aagctgccttccttcttgaaaaatgttaatgtctccagtagccctaagaartccataggctccattctgttattcaagatgccaaccaatggttttgacct

SEQ ID NO: 198 - DNA, 101 bp, Homo sapiens
ccgagttctggtaccatgactgtgccgttcaccattgttcttcagcacctrgcactgggctggcactcaacaagaacttgctagatcatgaagatgagcaa

SEQ ID NO: 199 - DNA, 101 bp, Homo sapiens
ctctgttagctaaactgaggaaccacaggcagggtggccttgaatttcagkctgaaggacccatcacccaagagtcttggcagcttcctcagcaaagatga

SEQ ID NO: 200 - DNA, 101 bp, Homo sapiens
ccagctgtctaaaaacatatatattttagagtttgttttcccaaataagayctcatacacggttcatccactgtgtttggttattgggtctctcaagctta

SEQ ID NO: 201 - DNA, 101 bp, Homo sapiens
atcacttccaggctaaatgtcacactcagatactcagctgcctacttactrgacacctctactgagatgtctgaattctggaccctcctcccaagccttct

SEQ ID NO: 202 - DNA, 101 bp, Homo sapiens
gggaagctctggagcattttgtgagcaccgtctcggtggatgggaaagccraagtctctgcccgtctcttactggaggcactaaaccccctccctgggttg

SEQ ID NO: 203 - DNA, 101 bp, Homo sapiens
agtcaccaccctggactatagtctgttgattttctacctctattctcttaytaaacttttggatacattccaaagcatcatggtcacttccagttatgaaa

SEQ ID NO: 204 - DNA, 101 bp, Homo sapiens
agcccagagacctctttggaaagattaccaaaccttgttaaaaacagacayccttggggccagacacggtggctcacgcctgtaatcccagcactttggga

SEQ ID NO: 205 - DNA, 101 bp, Homo sapiens
tgaagaaagtttaatgatggatttttgtttaagtatgcattcatccagaaracactttaactgttcttcagagagacatgatgtggactctaactgatgaa

SEQ ID NO: 206 - DNA, 101 bp, Homo sapiens
tcagctatcacaaaaaataaacgcaattctgaagatagcaatagctcataracatcaggtcaaatctgcaaagatgagcattgtcctaggtgctaaggata

SEQ ID NO: 207 - DNA, 101 bp, Homo sapiens
ttaggtaaagcgaaaaatgacagaattacattaacttgacaaatcaacacmgatagcaggaattttttcacacatttattagtaagcaattgtattagtcc

SEQ ID NO: 208 - DNA, 101 bp, Homo sapiens
gagctttaaaaaaaaaaatgcctggactccacccctaaagcttctgatttmattggcccatttgtttaactatcaatgacaatacagagagatgctaaagt

SEQ ID NO: 209 - DNA, 101 bp, Homo sapiens
aatggatgaaaagtaggattggtttgtttgttttcaggaagtgaggcaatygtaaaagggaaaaatgggaaaggcgaaacaagcaggatgtcttttttttt

SEQ ID NO: 210 - DNA, 101 bp, Homo sapiens
tgaagagggctatctgcctattccagactttatttccctggaaacaaaaargaatatgcacaaatcactgtattttggatttgaatattatatttaaaaaa

SEQ ID NO: 211 - DNA, 101 bp, Homo sapiens
aactcttgagcaaggcatcaagagttggtccttaccccacgcttggtacayttcagccacacttaaggtttaccgttccttttctcatgccatttcctcag

SEQ ID NO: 212 - DNA, 101 bp, Homo sapiens
cgtgagacctcatggttgtcttgtcagtcaaatgctctgaaaccccattgyctgaagctctaggttcaaactttgctccttcaggtgttcagagctgcccc

SEQ ID NO: 213 - DNA, 101 bp, Homo sapiens
tggatataagttcctgtttttctgattaatgtgcatgatcagacaagaaarttatatacaggaatcttaaactaatcattgctacagaaaagaatgggaag

SEQ ID NO: 214 - DNA, 101 bp, Homo sapiens
acacagtagtgtaatcctaatctttattgtgttagaaagttcctcaagacrtagatggaagtccataccccaggagaattactcataaaaatgaaatttcc

SEQ ID NO: 215 - DNA, 101 bp, Homo sapiens
ttcgatatgcatttattagcaaagcttctgaaggtgtcgtaagctgaacgygaggcagctgcctctagaagtgagattcacatgcagggtggaaatggtag

SEQ ID NO: 216 - DNA, 101 bp, Homo sapiens
gggccctttaaacatagccttgttttaataattagaccccccaccccagargagagagggaggaaatgaagcaaggcatccaccctcaggtgtaacatcaa

SEQ ID NO: 217 - DNA, 101 bp, Homo sapiens
atgatctgtgccaatactctgttcttcttagcataaaggtgaacagcaccyctgcactgtagcgtgaaagagtggatttgagtcttggctccacgggctcc

SEQ ID NO: 218 - DNA, 101 bp, Homo sapiens
agtagcagcagtttcacaaagactatctcatttattcctttaataatcctrggcaggaaattattagcagtcccattttatagctaagaaaactgaggctc

SEQ ID NO: 219 - DNA, 101 bp, Homo sapiens
tacatgggactaaactgataatggattataatttttatgacttttatttaraatattgctaattctttaatattttattttccagatttaaggaaactttt

SEQ ID NO: 220 - DNA, 101 bp, Homo sapiens
ggtctacgcactgcatcaaaatccaagctcagaaggcaggaaggcatctcycgcttctacattatccaagtggtgttccgaaatgccctggaaattgggtt

SEQ ID NO: 221 - DNA, 101 bp, Homo sapiens
ttattttcctaactccttgttacttcagtttagcaaattttttaaaaagtraaagtataaatatattaagacttttttgtaggggggctctggaatgtgaa

SEQ ID NO: 222 - DNA, 101 bp, Homo sapiens
tggacagccctggggctcctgctcctcccctacacatcaggcttcttcctrtggagctttctgtaccttcccaagccctcaatgaatgcaaaggaaaaaat

SEQ ID NO: 223 - DNA, 101 bp, Homo sapiens
ccaccacatacacagtaaacattctctcttctcagtggttgaagttgttcytgattacagctctcttatctgttctccctttgatttgctgactgatggat

SEQ ID NO: 224 - DNA, 101 bp, Homo sapiens
tgtgcgcatttcttatatcttcaatttataagtgcagaaattgagaatgaraggtctagaattaaacagtccaggattcaggatcttggttctgctactga

SEQ ID NO: 225 - DNA, 101 bp, Homo sapiens
gttgcttttcccaggaggtgtgagcctacctggaggaggcttaggcacagrgatacctgctggaggtctgagcgttggttgagcacctcctgtttgtagga

SEQ ID NO: 226 - DNA, 101 bp, Homo sapiens
caattatcttccatcatcaccctctccccaactggctgccgtttccacctrtgatagatcagtgttacacatgtgcattttccagaactcccagctgtgag

SEQ ID NO: 227 - DNA, 101 bp, Homo sapiens
ctgacatttactatatgccaaaacagggctgtttaaagttcatggtggttycatctactccttctgaggctacttcaaggtagggaggctacttcaaggta

SEQ ID NO: 228 - DNA, 101 bp, Homo sapiens
attctaggaaaagcacctgcagttattaatgcattaaaccagtgttctgamatgactaaatgcattatttctgctgtagaagaaaacgctgaggtgaggcc

SEQ ID NO: 229 - DNA, 101 bp, Homo sapiens
cacacgccaggcatggacgctttccattgttgtcaacaaaaactcatgcarctcaaatacttaaatgaattctcaaacatgtggttcacaattgaaaaaaa

SEQ ID NO: 230 - DNA, 101 bp, Homo sapiens
caactaagatcgtgtgccttgtgttggtggtaaagcaatatcagagccccrgtatggtaattctcaatctaatgcctgtctatgtgatcaggcttctcccc

SEQ ID NO: 231 - DNA, 101 bp, Homo sapiens
gaatttgataaaaacaagaaatagaagcataattatttttgaaaattacarttaaaactgttagaatcagaagcagaaaccattagcagcatagagagggg

SEQ ID NO: 232 - DNA, 101 bp, Homo sapiens
tctttctgagctttctgagctttgcaatccccagctcacccccccaacacrcccccacagtccttcttcccaacagttgccagcccaccctggccataaac

SEQ ID NO: 233 - DNA, 101 bp, Homo sapiens
atgacccactacaacttcacctcatgtatcttgaactttagggatatagcrccatttaaagagactaacctctcttggttcttgtcagtgaaactgggaag

SEQ ID NO: 234 - DNA, 101 bp, Homo sapiens
aaacttaaggtcagatatttcctcgagacatcagaagttaaagcccatgayataatgagtgaaaacatgcatagtaaactgtaaagctgtctacatatgta

SEQ ID NO: 235 - DNA, 101 bp, Homo sapiens
gtgtgttctttttagtttatcctttcatacatatatgtcaagtctccctarctcaattgtaagccctacaatggtaagggctatgttttatgcattttggc

SEQ ID NO: 236 - DNA, 101 bp, Homo sapiens
gcagagaaagacttctaataaaattccctccatatggaaggaaaaggagayatcgggagttacgttaatcatgctcatttcttaacagtgcaaatatcaag

SEQ ID NO: 237 - DNA, 101 bp, Homo sapiens
tccaaatggccaatctggccactccaaagtcccgcttccagactgaggaargggtgttaatgaagattccagcaaacaacagctctgtcctaccaactttt

SEQ ID NO: 238 - DNA, 101 bp, Homo sapiens
agagaactggagacaatgtagtataatattcggatgtacaaagtacaaacyataaagtctattttgttttaataattaacaaaggtgcacctagtacacac

SEQ ID NO: 239 - DNA, 101 bp, Homo sapiens
taagtacatgacattatctaatattggaaataagagtgcaaagccaaatcrtagccgtgtatagcagtgaatgttaggttgtcaggttcattcaaatgaac

SEQ ID NO: 240 - DNA, 101 bp, Homo sapiens
aggttaccgtgtatgtcaaggtcacccaggggaatgacttaggagtcaaaragcatggatcctactgcccactgtggtgtcaagttgctgttcacccttga

SEQ ID NO: 241 - DNA, 101 bp, Homo sapiens
aaattgcacgcaatgcatacaggaacaaagagagggtcaagatggttatcyttcctcctggcttccaacacaacctgctttgtaaaagccccacactgtta

SEQ ID NO: 242 - DNA, 101 bp, Homo sapiens
catgtcaacaacatctttcagaattggttttctttcacgatgtcgtccagytatgaaaacgagcctcacatgaaatatgctccaagccttttgagggcaac

SEQ ID NO: 243 - DNA, 101 bp, Homo sapiens
ctactccctctatgcttgtggtgattcagttgcagaaagacacatctatayttcatagctgtagaaaaattctttttttgtggttgatttcatgtggttta

SEQ ID NO: 244 - DNA, 101 bp, Homo sapiens
agtcaccagctggtgaccttgagcaagtctttagacctctctgagcttttycctcatgtgtaaaatggggacagacggagcccaacccaagatgttcctgt

SEQ ID NO: 245 - DNA, 101 bp, Homo sapiens
gtcagatgttacacaactttgcaatttccaatatgtgaatattaacatagrccaatgacattattacagaagcttactagaaatatattctgctggtcacc

SEQ ID NO: 246 - DNA, 101 bp, Homo sapiens
ctggcccaaatgccagcatttgctctcctgcctatttcccaggccgtggtraggggcttttcctcagggtcttcatggggagagtcaggggatgagtgcct

SEQ ID NO: 247 - DNA, 101 bp, Homo sapiens
agggagaagccagtacagaggccccagctagagtctgaatgaggacgatcmctctcccctgtcctggggagcctggggtcaccttgcagaacaagatggtc

SEQ ID NO: 248 - DNA, 101 bp, Homo sapiens
tctcccattttcctcctttatgctcctgccagttctgcaaatgtgggagtygcccaaggctttgttcatcagccctcttacctaatcacatttcttccaag

SEQ ID NO: 249 - DNA, 101 bp, Homo sapiens
ccaaggcaggcacctcctggtgctgccaaaaggcatcagaccccatgcccygctccttcctcatcctggactagaactgctttggggtggagacgttacct

SEQ ID NO: 250 - DNA, 101 bp, Homo sapiens
atccatttactgaagttatctgacatggctctcgagtcccttctaccccaygactccccttttttccctttatccttgtgaattatctgttgaagaagcca

SEQ ID NO: 251 - DNA, 101 bp, Homo sapiens
taaaaataaaatagttatgctatttacaagacacacctgttgaaataaggyagtgtaaatataaataaaagggtggaatatttatcatgtaaatgccaaaa

SEQ ID NO: 252 - DNA, 101 bp, Homo sapiens
tgtcagattatttaggcccaatccattctgttgattggacctagtataagyggaaggataaagatttctatccctacattaacacattttatgggttgcaa

SEQ ID NO: 253 - DNA, 101 bp, Homo sapiens
tctggagattcagctgaacacctggagagtctattgaggtctttgtccctygtctgttcagaatggcaccaggtactagcactgtataattttcaaaatct

SEQ ID NO: 254 - DNA, 101 bp, Homo sapiens
atacaaaaaagtagcaaaaagtgggatggggaaaataagattagataactrggtaataaccataaacgatgccctttttaagaaatccaattgttgtttat

SEQ ID NO: 255 - DNA, 101 bp, Homo sapiens
ccaaagaccttgttacagtgtttttaggcatggctcactttataaaggtcrtcacagttggccaagctatctggtatttattactcatttgatactcacac

SEQ ID NO: 256 - DNA, 101 bp, Homo sapiens
taagttctagagtgacagtggcttgctcaaggtcatatgtctaattcagtrgttccagggacaattggataatgtctggagacatttttggttgtcacaac

SEQ ID NO: 257 - DNA, 101 bp, Homo sapiens
atagggcattttgattattaaaactgtgaactgcttcctggaagggcaaayagaggtaactttggctgcatgttacaatccacaattcaatttggcatagc

SEQ ID NO: 258 - DNA, 101 bp, Homo sapiens
ctcagctctaaatgcactggtataactgttgccatttctggacatgccacrtgaaatttttcctttgctcatactattcatgcagtttggaattgattccc

SEQ ID NO: 259 - DNA, 101 bp, Homo sapiens
aaggtttaaggaactttcattttattagccagtggttaagtgcctgtgagmgcaatcatcagcaggtgcagtggtagaagataacaagcttcctaataaat

SEQ ID NO: 260 - DNA, 101 bp, Homo sapiens
ccccattttctgggcacaccccaaacatcttccatgggagaaattggtcaygtgagcccatccctgatgcccgaggagggatgggcttgccaaggctcttc

SEQ ID NO: 261 - DNA, 101 bp, Homo sapiens
cctccctgggaatgacaggttctgtttttcccttcaactattttagcacakggagttcacaactcattccagctacaatgggaaatgtttagtcccgactc

SEQ ID NO: 262 - DNA, 101 bp, Homo sapiens
atgaaatggaacaaggaaaaagaaagattagaatacatgtgaaacctctamaatttttaccatatagagcaggaaagaaacataatctaaaccatattttt

SEQ ID NO: 263 - DNA, 101 bp, Homo sapiens
taaccgaaataccctgtgtgtgtgtgtgtacatatgatcgagccagcctcytcagtgccttgcattgctgttaagaggggaagttctaggctaagactttg

SEQ ID NO: 264 - DNA, 101 bp, Homo sapiens
gccttccatttttaagcaaacattttacaagcttgtactcattctctccaygttgtattaagttttatatttgacattgtatttaaagcatttaccatatt

SEQ ID NO: 265 - DNA, 101 bp, Homo sapiens
tgtgaaaaacattgttagcttgaagaatgtgcaaaaacaagctgtgtgccygatttggctttcaggctgtagtttgccaacttgtgacctaggccttgagt

SEQ ID NO: 266 - DNA, 101 bp, Homo sapiens
gagattgtgtcttaaaaagttttgctctctcctcagaacctagctcatttrgtaacttgttattgctgaataaaaaccaatttattgataaatgaatgtca

SEQ ID NO: 267 - DNA, 101 bp, Homo sapiens
atatataacatagatagtattttttcttgtatcttagtgttctgagttcamctttcttcttctcttcttcctgaagtacatacttgaaacctcattcacag

SEQ ID NO: 268 - DNA, 101 bp, Homo sapiens
ttgtggtaggctgcttaataattaattccctcacctcagtttttgaatgtygttctgtttatgcctcagtatcaaaaacaactgagaaaggggccgcagct

SEQ ID NO: 269 - DNA, 101 bp, Homo sapiens
cttaatatttggctctgtgtccccaaccaaatctcaccttgaattgtaatratcctaacgtgtcatgggaggtaaatggtgggaagtaattgaatcatggg

SEQ ID NO: 270 - DNA, 101 bp, Homo sapiens
gatgaaaaggtcctatcttatcatacacctttaccataaacttcccctccygccacccccagaaggaagagctgaggcagtttccaaaggtgcctgacttg

SEQ ID NO: 271 - DNA, 101 bp, Homo sapiens
gcagagcgatggttcagatcccaggcaggaaggagatggatagcaaaagaktttatcacactactcagaattgtgcttaatttaaaacttttaaaatattc

SEQ ID NO: 272 - DNA, 101 bp, Homo sapiens
tttatccaaagaagggaaatcagaatgatgaagagatacttttcctcttayatttttaggtttatcaccttcatattgtcaaagcatgatgccaataacct

SEQ ID NO: 273 - DNA, 101 bp, Homo sapiens
ctctgcaatttgagtttgttgtgttctaaagaggtacaaaaaaacatgcarctggttagcagcatgctccagagacccagaactgccccagaatgatgggt

SEQ ID NO: 274 - DNA, 101 bp, Homo sapiens
gccaatatccaagacagacgttcaattttccaaaaagcccaagaaattctraaaagtggcctcacaaacaggtttttctgaggcttagacaaaaattcaag

SEQ ID NO: 275 - DNA, 101 bp, Homo sapiens
ttggagaaatgttaattcactctctctagtgtcctgaaatggattggatgrtgcagtatgttgtattgcatggctcctaacccaattccagggagtttctt

SEQ ID NO: 276 - DNA, 101 bp, Homo sapiens
gtacttaggcactaattggcatttttcaacatttctgttaatgtagaacaygtctttcgaaccctcaggggccttgctttggagctaatgaaaataaagca

SEQ ID NO: 277 - DNA, 101 bp, Homo sapiens
tttggggatgtggagggaaagcgagctgggagctgagcccagaccagctcyggtaggagtcagaagaatgtgccctgctgccagtctgagggtcaaagtgc

SEQ ID NO: 278 - DNA, 101 bp, Homo sapiens
tggttaatcattcactcaatcatttgataaatatttgccaagaactgtctrtgtgtaaggtacataatagacactcatttatgtgattatgaatccctcta

SEQ ID NO: 279 - DNA, 101 bp, Homo sapiens
acctctcctacattctaaaagaatggcctgaactatccatgagaacatgayatccgaacttgtaaacttatttccctcatcacagcccataaagaattata

SEQ ID NO: 280 - DNA, 101 bp, Homo sapiens
tgcaacttggtaaaaatattttaacttcatatgctacgaatttgattttcyttgtattaactacacatgtaattagatttttttctttccaaatcatcttt

SEQ ID NO: 281 - DNA, 101 bp, Homo sapiens
agagagatccctgtctctcctcttcttataaggctacccatttttatcaarttagtactccatccttatgaccccttttgatttttttttcttttgaaaag

SEQ ID NO: 282 - DNA, 101 bp, Homo sapiens
ttatataaagggatcttacctctctggatggaagagactgaaatggaattrccaaagtccaaatatgtgtatctgttgcatttaaagtagcacagtttctc

SEQ ID NO: 283 - DNA, 101 bp, Homo sapiens
ttcacctcccaaaatgttgggattacaggcgtgggccactacacctggccrtaagtacagtacacgtcacccctgcttgaaaaatcatcaaagcctttcac

SEQ ID NO: 284 - DNA, 101 bp, Homo sapiens
tcgaaagatttacatagttttagaaaggaggaaaggcaaagagggagttgrgaaatgaaagaaacagggagaagacatggcttctaaattcagggttggga

SEQ ID NO: 285 - DNA, 101 bp, Homo sapiens
taaattgcctgagagcttagagacaatcaggtcaccaccgccctcacaagrgaaaagcttcttacttccgagcagaacggttcagctgggaagagaggaag

SEQ ID NO: 286 - DNA, 101 bp, Homo sapiens
atttccaagacaatttttcatcctttcgtataatattccaggtttgttggkgcctcttctctgtatttcccagaaaataattctaccctctggagaactgt

SEQ ID NO: 287 - DNA, 101 bp, Homo sapiens
aaagatgtggccatcaaggagaagtctttcccatcgtaaatatccaagggygtgactgagccatcactgaactggacccagcaactgatggctgcttccta

SEQ ID NO: 288 - DNA, 101 bp, Homo sapiens
ttgtccttgttttaaggatcttcctgcaggatccactccctagcacttctkgatggcctggctcagggaaatcttcaggaaagagacccaggcttgcacta

SEQ ID NO: 289 - DNA, 101 bp, Homo sapiens
gtttttgctttgaggaaacttgatatgatgttaaatttctaaaagggcaargaaagtagaattgatcaggtagcagaaattttacacagttttggacatca

SEQ ID NO: 290 - DNA, 101 bp, Homo sapiens
tgcccctaccctgagtgctgagagtagaactattgagagacctctttatgmgaaattttcagaaatccaacatggttcttggtctagaaagtgggatcaag

SEQ ID NO: 291 - DNA, 101 bp, Homo sapiens
gtggtcacatttatctgcttctttgtatttctactaatcgttctattagakgctggacattatggatatcctgttgttgcgtgtctggattttgggttttt

SEQ ID NO: 292 - DNA, 101 bp, Homo sapiens
caaataaaatattttttcttttacatagtacatgaaagtaaatctaatctkggagctcatttaggatgctgagcagagtaactggagttagactataagat

SEQ ID NO: 293 - DNA, 101 bp, Homo sapiens
aacaggctgaggttcagtaagctgtcatagctgagctgagacttgaatgcmggtcagatttcagaatctgggctcctcgcacttctcaccacactgcctgt

SEQ ID NO: 294 - DNA, 101 bp, Homo sapiens
ggactctccaacagcataaattggctccagcccgcaagcccaactttccckcagctgagcccctttcagacttctgcccctgcctctgatctatactttat

SEQ ID NO: 295 - DNA, 101 bp, Homo sapiens
cttaatctatttagactgactacagggatctttgattgcctaaaacaacartatagcaatttctctatctgctctcgtcttcctcccgtcatactcataca

SEQ ID NO: 296 - DNA, 101 bp, Homo sapiens
ctcttttgatatccccttcaaaatgtctgctccacacacagagcatcacayatgtggtttatatgtagctggctgaatttctttcctttctctctttcttt

SEQ ID NO: 297 - DNA, 101 bp, Homo sapiens
gatagcgctattaactgtttacacagtaagcacaattttctattctctctytctctctcactggtttcaaagcagccaaaagctttgagccccccagcaac

SEQ ID NO: 298 - DNA, 101 bp, Homo sapiens
ataagctgaaccgagacctgcttcgcctggtggatgtcggcgagttctccraggaggcccagttccgagacccctgccgctcctacgtgcttcctgaggtc

SEQ ID NO: 299 - DNA, 101 bp, Homo sapiens
ggaactttcaagcttgtgttggggacatggatctctataagtaaccacatrtaagtgtaacaagttttgatatgaaagaaaagaacagagtgccctacaag

SEQ ID NO: 300 - DNA, 101 bp, Homo sapiens
tgccacctcattagcaaagttcctgggagccactgacatggaagaccccckgtttccgcctctcggtttccgagcctcagaaagatggactgtgaggcctc

SEQ ID NO: 301 - DNA, 101 bp, Homo sapiens
acatttctatggggctagacttttccttgtcaagattataatttttcttaygagtttttacctgaaacccctattttctaagaccccatggttaatgagtc

SEQ ID NO: 302 - DNA, 101 bp, Homo sapiens
ataagccgtgggtgtaaccatgtcccccacggagtgagaaggggagggtcytctggtttgttactttctgctcatgaggcggggcgatggggagatgcctt

SEQ ID NO: 303 - DNA, 101 bp, Homo sapiens
ctcaaataaagagaaatttaaatcaaaatgacttggctttgtagagtactmctaattttgatttttgtaatcatttcatcttcctatatatgtccttttac

SEQ ID NO: 304 - DNA, 101 bp, Homo sapiens
gaagtgataggtggaaatgataattgttctgtaagagatattctaaggggyaatttaaaacatgtcaatataggcttcttctaaggtggtaaactcagctt

SEQ ID NO: 305 - DNA, 101 bp, Homo sapiens
actcactaacttattctttgtaaaaaggagagcaggtgcacaggtgtagaracaagaaacaacttggagagtgttggcgttgctggagcaccaagtagaaa

SEQ ID NO: 306 - DNA, 101 bp, Homo sapiens
ttcagaacttacgttagtagagtttgaatagttaagacttgaaattaagayccttgctttagtacataatctcacaaatgactttcagaaaatggtgcatc

SEQ ID NO: 307 - DNA, 101 bp, Homo sapiens
gaaattgctgggccatacatagcgatgcgtttgtaaaccagctcactgaayaagaaagccttgattagcatttgctaacatctgtgatgttaatactccta

SEQ ID NO: 308 - DNA, 101 bp, Homo sapiens
ctgacaaccagaactcaagtctctaaccttctctgctgtcccagtaatccrtgcctgccttttctctgccttcagccctttttgctccatcagtactttta

SEQ ID NO: 309 - DNA, 101 bp, Homo sapiens
gtcatgcggcttgctaatgggtttcaaggagcaagctgcaaagagcccctrgacttgctctgatgggtttcaagggacaagatattagtaacgcactcaca

SEQ ID NO: 310 - DNA, 101 bp, Homo sapiens
acactgtgctccgcttttcctcttagcctcttcccctcaacgaaatggtargagttcagctgacaacagggtaaacagattattgtgttattgctggctga

SEQ ID NO: 311 - DNA, 101 bp, Homo sapiens
caaataactataaaataaactcaaaatctttttttcctgcattagttcackgaaaataaaaagggttagcaattagaatcaatagattctttgaaaacact

SEQ ID NO: 312 - DNA, 101 bp, Homo sapiens
attatcatactgctaaacaccatgaaacactgtgtaagtttgcgctattayagttattttaaactgtttttatatttagttgcttacttttaaatttatat

SEQ ID NO: 313 - DNA, 101 bp, Homo sapiens
aaataagcttggacatgaccttttttagcataatgactactgtcatttcartgtcaacctttgaaagcatccattcttgttaaaaacatttgccactgctg

SEQ ID NO: 314 - DNA, 101 bp, Homo sapiens
gggtttacactgctcccctctgctagagcatggactaccagctgacctgcmgagtcactcaccttaaatgttagcagtagctatggggtgtgtgtgtgtgt

SEQ ID NO: 315 - DNA, 101 bp, Homo sapiens
attagttccacaacaaactagatgtagtattttgcatatatttcccctgcyaacgcacctgtggtagtttctagtacatggtttcacttctatgatctttt

SEQ ID NO: 316 - DNA, 101 bp, Homo sapiens
tccagcatattcccagctgtagtggctacggtaaaagactcattctgtatyagagcagacggaatctagaaagacagccatcatctacaagttgggtttaa

SEQ ID NO: 317 - DNA, 101 bp, Homo sapiens
ctgaacagactgtgctttagagcctctggaagacacccaacagaatgttcygaaaaatgcgattatttttacacaaaattgccaatgtaaattcaacttct

SEQ ID NO: 318 - DNA, 101 bp, Homo sapiens
tgctgtgtgatgaggaagccaagaactgaactgtaacccaaacacaaacaygttgcattgccaggaaatggctaatgcggcctcccattacacagagcttt

SEQ ID NO: 319 - DNA, 101 bp, Homo sapiens
ttctaaagtcatccatccccttgacttaagctccaggatggatgcagacayggacggacgcctgtgcacagacaggagtctggaagagcacctgagccctg

SEQ ID NO: 320 - DNA, 101 bp, Homo sapiens
tttaatggaaagttaattgttatgcaaatatgcattcacatgttattttgyttgtttgtttgtttgagacagggtcttcctctgtcgcccaggctggagtg

SEQ ID NO: 321 - DNA, 101 bp, Homo sapiens
actccaagtgctataagcctgcaatggactgtatgtttgtccccctccacygcaaatgtgtatgttgaaatcctaacccccaatgtgatggggtctttggg

SEQ ID NO: 322 - DNA, 101 bp, Homo sapiens
tgaacttaaacccgagtatactagaaatataaattattatatacaaatggrtgtcttttacagcaatagactccagcctaaattgatggtaggggttttat

SEQ ID NO: 323 - DNA, 101 bp, Homo sapiens
ctttactatttagtctagcctgggattctgtatgtgctggctaactgcaamcccgaacaggcaggccttggtgtgggattctctagttgagctgggtcact

SEQ ID NO: 324 - DNA, 101 bp, Homo sapiens
tcttataataaagattattgttattattataaccacctttcagtgtttctrtcttaccctcacatcttcacttttcccctaatctcaagatagagtggagg

SEQ ID NO: 325 - DNA, 101 bp, Homo sapiens
aagtggtaaggttgtttgtctgaggtaggtgattaatagacagccttcctyagcacgtgcaaattaaaatagaagaaggaattatgattggagctctcctt

SEQ ID NO: 326 - DNA, 101 bp, Homo sapiens
cctgatcaaccttcaaaggaatcctcctgagtttacatgagttggaaaatrtgttttcctggctcgttaaagtggaaccaatctcctccgtgtggtagaga

SEQ ID NO: 327 - DNA, 101 bp, Homo sapiens
cgggatatagtagccatgaggaaaacaatgagggctacccttacagcaccrgactccagatggtcttcagtgcattctttgggtagcagctccccaggagc

SEQ ID NO: 328 - DNA, 101 bp, Homo sapiens
gacttgttcaaggtcatataagcagcagtggagtccagaagccaggtttcygtatgccctcttccacatcacattgcaagacaccctctgaaaacactcct

SEQ ID NO: 329 - DNA, 101 bp, Homo sapiens
ggtccaccctctcagttaggcagtagtaaaagatctaaacataatcaatcrggcacattgtatgtagctgtgagggttagaagtacaaaatgtagttgtga

SEQ ID NO: 330 - DNA, 101 bp, Homo sapiens
ggaaataagctcatagctggacagacagcaacgacatagatccggtggagrtgaatctgcagatagaggataattggtcttggcttcaaggatggaaagaa

SEQ ID NO: 331 - DNA, 101 bp, Homo sapiens
acatatgcataatgatcctcaattacgtgccaagcattatggaagtcatcrctaactcctctgtcacctttactttcctgatagcacctgttgatgctgtc

SEQ ID NO: 332 - DNA, 101 bp, Homo sapiens
aaaaggcccccagggaggaattgatcaaaccaaaatgtggatgagtagatrttaggcgaacaccaggcaaatggtggtgagagaagggagcaaagtgtatt

SEQ ID NO: 333 - DNA, 101 bp, Homo sapiens
aaaataatctaaatcttattgagcatgataggattaagtgggaattggacmgatagtggagttggggatggattgtaattatactacactgcgaaaaagca

SEQ ID NO: 334 - DNA, 101 bp, Homo sapiens
ctactttagccactctcaaaactttgtgataaatctgcaatagaggtattrtatatacatgcagaaagctgtgggaagcccagaggagtaagtgactaacc

SEQ ID NO: 335 - DNA, 101 bp, Homo sapiens
acaaaataattccttcttaaaaattatgtattagaaaacttttcaaaattyatcccatcctccagaaaccaataaaataacacacactagaggtccttcag

SEQ ID NO: 336 - DNA, 101 bp, Homo sapiens
cagagctctaccaatcataacagagaaggcatggaaagctggtgaaaatgytggaacgagtttctttttacatgttgttcaatttttatttttgcaattag

SEQ ID NO: 337 - DNA, 101 bp, Homo sapiens
cttcccccaaaggccctggaaactatcattctactttccatctctatgaakgttatactctaagtacctcatgtaagtggagtcatgcagtgcttgtcttt

SEQ ID NO: 338 - DNA, 101 bp, Homo sapiens
gtaaatttattgcttgctcaatccttccttgtatttcattagcatattgcyactctacacttgtcctgtatttagatatttccttcctctatggtttgttc

SEQ ID NO: 339 - DNA, 101 bp, Homo sapiens
aaaccatggggttgagtgcaggtgggataacaatgtagagattggcaaacrtgatgtggaaggtgcgagagacattgtgtccaaagcgatgggcgaggatg

SEQ ID NO: 340 - DNA, 101 bp, Homo sapiens
cttaacatatgcaaaatgaataagtgacaaccccaaccctcaccattggcyccttagaactgaaaataatggcagttgcagtgtttaagggcaacatgaat

SEQ ID NO: 341 - DNA, 101 bp, Homo sapiens
gataatgactgggaattttctagaattggaaatcctcctgtttgggaccaygaagaatcccaggtaggatatgtaaaactaaatgcacatctggcaatatt

SEQ ID NO: 342 - DNA, 101 bp, Homo sapiens
aacaaaacaaaacaaaacaaaacaaaacaaaacacctcttattctagaatrttatgcttcaggagagtgtagctctcctagttttagtttggttcagaaga

SEQ ID NO: 343 - DNA, 101 bp, Homo sapiens
ggcgttcagccctgggctgtgctgtattcagggctctaaaaacgctggccracttgaatgtgtgaatacagttatggcagggagggaggggaggtgctttg

SEQ ID NO: 344 - DNA, 101 bp, Homo sapiens
tttgtgcatactgtgatgattttagaaggtaagaatgtcaagctgtttgarctgaaagtaaagatagccccttatcaggaaagtgccagccacccttgctg

SEQ ID NO: 345 - DNA, 101 bp, Homo sapiens
aatgttgatgcatttaacagcttagattaaatggacaaaatttatgaaagrcacaaactttcaaagcttactcaagaagaaaaagataaccagagtagccc

SEQ ID NO: 346 - DNA, 101 bp, Homo sapiens
gatctcgactcggagcttcttgcccctcttctgtggaatgaaaggggagckaaggaggagggtgtctgaggggcgagagatgagcctggaagagaagcaag

SEQ ID NO: 347 - DNA, 101 bp, Homo sapiens
cgttgttgcataggactagactaaaccaagcgagctgcattccatgcgaaytattctatcgtggggatcaagatctccagctgagaaaagatgccaccaga

SEQ ID NO: 348 - DNA, 101 bp, Homo sapiens
tgatattactaactggaagtcctctatagaatgcttttaccatgatgtacrtagtctgtctaggattccttatgggaaacatacctaaaattgatggattt

SEQ ID NO: 349 - DNA, 101 bp, Homo sapiens
atcttattctgaaagcagatggggcatcagaaacatcaaacaagttaaaaycacaggaattaaattataaattttaaactcccttttattgaaatataagt

SEQ ID NO: 350 - DNA, 101 bp, Homo sapiens
gctgtagatggctataaagcggtccaaagacatggccagcagcacagctgrctccatcatggataaagaatggatggagaacatctggaaaaagacaagct

SEQ ID NO: 351 - DNA, 101 bp, Homo sapiens
gccttagtggggtttcaggagggagcagagataaaaacacatgtcttcaakccatcatcttgaactggaaatcctaaatatcttttgattccttcttttga

SEQ ID NO: 352 - DNA, 101 bp, Homo sapiens
cagggaatgtttcagaatgaagggagggtacatggataaatcagtcagttmaaatattggtgagccccctgcagcacgcgcagatctttgcttaggtgtaa

SEQ ID NO: 353 - DNA, 101 bp, Homo sapiens
aggaagtacggcatagcagttaggcactcaggcatggattcagaaatacgyggaattcagtagggctctggcacctactaacaatttggttactctccctg

SEQ ID NO: 354 - DNA, 101 bp, Homo sapiens
gcactcaataccctgaaaattcgctcgtctctcatgggcctgcctctgaarctgctatgaaagccggcaaccacacagaatttgcctccggtaagaattat

SEQ ID NO: 355 - DNA, 101 bp, Homo sapiens
ctaagtatgatgtagccctctgtaatgataatagtaatagcaatagccagmactccagcaatagtaatagccaccactgacttcattgttaactacaggcc

SEQ ID NO: 356 - DNA, 101 bp, Homo sapiens
gtgagacacacacagagtctgcacagcatctggctgcggggtggattatgrttagccaagggttcctttttatggatgactgcggtagtgaagttgcagac

SEQ ID NO: 357 - DNA, 101 bp, Homo sapiens
acgataatagctcctgtgccaaagaccctgggcagtgtcaggatagctgtrtagctcagtgggctgtagatggctataaagcggtccaaagacatggccag

SEQ ID NO: 358 - DNA, 101 bp, Homo sapiens
aaaactataaaaagagacaaaaattgtgattatgtattgaatgccaaaggrgtcaattctgcaagaaaaataataattgaaaatatatgcaccccacattg

SEQ ID NO: 359 - DNA, 101 bp, Homo sapiens
ttgggcagagttctgtgcgaggggcagcagaggatgcaaaggcctataatytccctgtcctctttggcgcttactgtccactgacagggaggcagaatgac

SEQ ID NO: 360 - DNA, 101 bp, Homo sapiens
ccaaaaaacggttgggagcaactgctctagaaatttgttgtcttcataaaygtttctgactcttagtttctgtttttatcccttctctaagtaccaacttc

SEQ ID NO: 361 - DNA, 101 bp, Homo sapiens
tattctttctcatcttccaaagctatttcatcctccaaagtgtttgttatrtacttttgaatgaatcacaatataccaataccaacacatattttcattat

SEQ ID NO: 362 - DNA, 101 bp, Homo sapiens
ttggtttccattgataatttggaggcattgtcctctgtggagttgtgtcayctatcagcgggctattaatttagggtatggttatagacaactgcagatcc

SEQ ID NO: 363 - DNA, 101 bp, Homo sapiens
gtggatttacttgcttggtttccattgataatttggaggcattgtcctctrtggagttgtgtcatctatcagcgggctattaatttagggtatggttatag

SEQ ID NO: 364 - DNA, 101 bp, Homo sapiens
aaaagctttatatccttacatgaaggacagaacaggcagctatatggtgargaaatgtacagacacaaatatccatatattgaataattggctggctgggg

SEQ ID NO: 365 - DNA, 101 bp, Homo sapiens
atctccgcgtcttcttcttctgtgtgccccagatataaataagcctctatygtatcgctggaaaaacaaactcaccaagttctatattaggcctattgcac

SEQ ID NO: 366 - DNA, 101 bp, Homo sapiens
ttgtgcacacctattacaggaatggaggactcctgtaatgtgtctattagycttaattcgggctccattatacattcctattctgttccctcccctttccc

SEQ ID NO: 367 - DNA, 101 bp, Homo sapiens
acaggctgtcaaatgagagcacgtacttaagaggctaacacagtatgaccrtatgtggcaataaatgagtgctgagtacatgtctatttcttttccagtct

SEQ ID NO: 368 - DNA, 101 bp, Homo sapiens
accctcacagctgctcccactggagccaggctcttgcctggaagaactgcrggttccctgggagactccccagagcccctccttagtggacccaggcccac

SEQ ID NO: 369 - DNA, 101 bp, Homo sapiens
cagtgattacctgcactttctttctctgacttctttggttagctcttctgyttattgaaacaggtaagcagagaaaagtatttaaaaataatctctctctc

SEQ ID NO: 370 - DNA, 101 bp, Homo sapiens
gtaacacaactacataatatccaaagacaaagtagaatggcaagaacttcrcagagcggaataagccttgatggtaaagggaaacatccaaataagcaagc

SEQ ID NO: 371 - DNA, 101 bp, Homo sapiens
tcatcatcttcttgctgcccaagcctctgttcagtcccccaccagatgcgkcattcaagttgtaaagcaaatgtactatttcttgacatttctagaaaact

SEQ ID NO: 372 - DNA, 101 bp, Homo sapiens
gtttgagtcatggttttggaaaatcacatgatccataccagaggagagctktgtcttcaaattatcttctagaaaggttcaccagaaagtacaaaaatgtt

SEQ ID NO: 373 - DNA, 101 bp, Homo sapiens
taagtcttgaatttgggtagtgtgaatcctccatatttgtttttcctcttmagtattgttttggctattcttggtctcttgtctttacatttaaactttag

SEQ ID NO: 374 - DNA, 101 bp, Homo sapiens
cagtggtaaccaggcagtaagtaccatggattttggatgagactcagtacmttgctggcatcatgtgcaacccagcacattcccagctctggtggccacag

SEQ ID NO: 375 - DNA, 101 bp, Homo sapiens
tgtgtgtgtgtgtgtgtgtacacatgtgtgtgcgtgcatgctttttcatgrggcacacttattttcagatgttcacatggactctttttgagattccccag

SEQ ID NO: 376 - DNA, 101 bp, Homo sapiens
caatgcaagggatttgtaaagaaacagggaaatgaatgatctgacaggccrtttgttaccaccaacatttttcttaatttaacctgaacttacttgctctt

SEQ ID NO: 377 - DNA, 101 bp, Homo sapiens
atccatgcaatgcaataaacagccatagacagaagcgaagcgctgatccaygctacagtgtggagaaatcttgaaaacactagggaagtgaaagaaaccag

SEQ ID NO: 378 - DNA, 101 bp, Homo sapiens
tataccaaggatagtttgtgcagttacaccggaaataagatatttcctgcrtttacagacatctacatgcttgcctttttttccatttcccactgaaccag

SEQ ID NO: 379 - DNA, 101 bp, Homo sapiens
atgggggatgagacaaagaacttcatgggtgcagcaggtctcttggtgtcrtgtgggaaacacaagcagaatcagaagttcccctggcctctccctgggtc

SEQ ID NO: 380 - DNA, 101 bp, Homo sapiens
aaagggagaatggggtggagggccagaaagcaggagtgccatagagtcagkaagtgaaaaattgcaaatgtgggcaatgtgattaggcaactgggtgtgta

SEQ ID NO: 381 - DNA, 101 bp, Homo sapiens
caccctagaaatcctggagggaggaccgaaaggtagcatggagtcaataaygagcctctttttatttaactatgattacatgtcaatcaatgtctgattct

SEQ ID NO: 382 - DNA, 101 bp, Homo sapiens
cttggcatgctagttaacccaagggatggctctacaatgccttacagtttrtaaagtacttccttctgtattatttcatctgaccttcgcaataaggctat

SEQ ID NO: 383 - DNA, 101 bp, Homo sapiens
aaatccacagccattcaggtggcttatgttactggcacttagcattccgcraccatggtccccagaggctctgtggacagaggtgccctgcagttcctttg

SEQ ID NO: 384 - DNA, 101 bp, Homo sapiens
aacagccttattctttcttatttccagtaagtattccaaagaaaaacatgytgactggcccagctcacttttgcacatctctgggtcatgaatctatgtct

SEQ ID NO: 385 - DNA, 101 bp, Homo sapiens
taatgcatctaaagttcaggatgtataatgaaatctaggaatgtgaactaytcaggagaaaaacagacatgatctaagagttcaaaagaaaaacattagca

SEQ ID NO: 386 - DNA, 101 bp, Homo sapiens
gtgagatcatggacttgggccccctaggccagcccagtctctttgcagccraggaaagtgaggcttagctgtcgggggctgtggggggatgcagcttgcca

SEQ ID NO: 387 - DNA, 101 bp, Homo sapiens
ctacactaacaccatgagataggtattcttattagcatcagtttttcgaaygagtacttcaagtttcaggaaagtaaagaaacttccctgaagacagtatc

SEQ ID NO: 388 - DNA, 101 bp, Homo sapiens
ttctttatcattgaatttcaaaatctttactaggacaaatcttggtggtargctttctatatcgaatttccctaggcacattttgcttttgcgatttgcag

SEQ ID NO: 389 - DNA, 101 bp, Homo sapiens
cagggtgtgtccacactctgctcacaggtggatccacggctttccagtgcrgagagtcgagatgctccctgcagcccaggccccgggcacctcctgcaacc

SEQ ID NO: 390 - DNA, 101 bp, Homo sapiens
tacagaaaattgccaacctctggaagcctcagcaggaccaatgtcctccaygcagagcccttcttatcccctaggaccgcaggcccaggctcctctggggg

SEQ ID NO: 391 - DNA, 101 bp, Homo sapiens
actgaaactctctgcccacattccacattctccctctccccaacccttgakaaccttttctttccttctctccttcctttcctctttccctccttccttcc

SEQ ID NO: 392 - DNA, 101 bp, Homo sapiens
ttctgaaccaggcaaaggatgatggggaatgcagtcttacgacgtgatgtygcgtttagagggttttcatcagttttaatgaaatacaaatgcacccaaag

SEQ ID NO: 393 - DNA, 101 bp, Homo sapiens
ctgtccccgtcgtccttcctatgctcacggcagtcacgtgagcctaaagargtcatgaaaggaacatagcgaccactccatgatgtggattaactcatcct

SEQ ID NO: 394 - DNA, 101 bp, Homo sapiens
actggacccagcccagcccagctctttccactgctcacctgctgcccctgygtttccagggactccacgctcaccagggacacctcgctctcccttagggc

SEQ ID NO: 395 - DNA, 101 bp, Homo sapiens
cataaataacaaaaagtctactaaaacagataccttgggatagatttattrtgccattttaggatttcactttcaagttgcttaatagaaaatcagtgact

SEQ ID NO: 396 - DNA, 101 bp, Homo sapiens
aagaaagattttgatacagaggcacacgcagagggaaaacagccatgtgamgacagtgacagaaactaaagtgatgtagctccaagacaaaaaatgccaag

SEQ ID NO: 397 - DNA, 101 bp, Homo sapiens
actagttacaaggcagaattatctttctgattgcatgaaacccatagatcrttttctctccaacagaaatcttttcagtaacctcaatccacgttttggct

SEQ ID NO: 398 - DNA, 101 bp, Homo sapiens
acagtgtctgcccaggtcagacactgtgtttagaattgctggtgattttgkagttcagaattactggtgattctgtgtctccatccttcttcattccaaat

SEQ ID NO: 399 - DNA, 101 bp, Homo sapiens
ctctattacaaagataaaatggcaagctacagagtggtagaaagtatttayaaaccacacgtctggcaaagcacgagtatctagaatacacaaagaatttt

SEQ ID NO: 400 - DNA, 101 bp, Homo sapiens
atcctaacagaagtcacatggctttatttcatggccagaaccaccaggctrttacaggaaagccaaaaagaccagacagagaagaatgtttccttacagta

SEQ ID NO: 401 - DNA, 101 bp, Homo sapiens
ggtgacagccatatgctcctgatcacaagaagaaattatatcgggtccagyggcggctgtcacaaagccatatggggtggcatggcagccttctgcaggtg

SEQ ID NO: 402 - DNA, 101 bp, Homo sapiens
ctagtaaccctttgtgaggctacaaaaaaaaaaggcatatttgcttgcccrgggggcttctcttccagttcacctgggtagaattctgggtgtagtcccca

SEQ ID NO: 403 - DNA, 101 bp, Homo sapiens
gtaggacttactttgtgcctgagttcagtgaccttgtgctcactctcttamttctccttcctccctggctggccattccttctcagtttgctttgtaactc

SEQ ID NO: 404 - DNA, 101 bp, Homo sapiens
tttgcttcctctctcacaatgtgatctctgcacatgttggtcccttgtcamcttctgccataaggagaagcagcctgtggctcgcaccagaagcagatgct

SEQ ID NO: 405 - DNA, 101 bp, Homo sapiens
agtgttgttctgtgttattattctctaatgtagaatcgcaccatcctgggrgtcaggcatcttccgcctcctctttgacctagtttgtggcacacagcagg

SEQ ID NO: 406 - DNA, 101 bp, Homo sapiens
aagtgaaacttaaatcttgaatcatgagtaaaacgtaccaagcaaaaaacrgacaatttgatctttgacgaacctgacacaagcaatggggaaaggattct

SEQ ID NO: 407 - DNA, 101 bp, Homo sapiens
atctgccttctagtatgtgaggcaaccttcatcagcatgtagtagcatgtyggtgctggctagttactttccaagagggagataaacacctcaaaataagc

SEQ ID NO: 408 - DNA, 101 bp, Homo sapiens
tagtgaggagtgagaattatatcacaggatttttgcaaaagctgtaataakataactaatactactgcattttgttcccaacattcacaattgaagaaaat

SEQ ID NO: 409 - DNA, 101 bp, Homo sapiens
aaataaaaagtcataaaaagaggaaagaataaaaatttccattcaataggrattgatcttaaacatagatggagggatcagacaagggaagtcatgtgatt

SEQ ID NO: 410 - DNA, 101 bp, Homo sapiens
acaagtggttaggtagacagaagctatcgggaacattctggactgctggarattgctatagtctcaacattttctaagacagtcgggtatagagctttgta

SEQ ID NO: 411 - DNA, 101 bp, Homo sapiens
gttgcagcccccctgagcccccattcacaggaggtctcctgctacattgamtataacatctccatgcccgcccagaacctctggagactggtgagtaaggc

SEQ ID NO: 412 - DNA, 101 bp, Homo sapiens
ggagtaaggtaagtatgcatggctgacttgaaaagatactttctatatacrttgcttaataaactatcaaattgctgcagaatgatatatgtggatgagat

SEQ ID NO: 413 - DNA, 101 bp, Homo sapiens
atataaggcaaagctcataaccatcctccagtgttcaggctcagcataagycctctaggaaacctttgtacctttctttgggcctcccccaccatagccct

SEQ ID NO: 414 - DNA, 101 bp, Homo sapiens
gtccttaaaaggaagggagctcccgtattcccctcttcttccttcctctgkgctggcatatgaacacaatgactggaagctgaggagtcatcctggatcat

SEQ ID NO: 415 - DNA, 101 bp, Homo sapiens
ctcggttgtcctcaagcaaaaggaatgctatcaataagccttcctaccacrtattgaaaattaaagtccttcctttttacactttaagaccttctaataag

SEQ ID NO: 416 - DNA, 101 bp, Homo sapiens
ccctaattgagaataaatctgtctgaggcagatgtttggcaaaagtagtgygagtgggttttcgttaggtcttttaccgttcttagaaatgctgtcagcat

SEQ ID NO: 417 - DNA, 101 bp, Homo sapiens
ctgcctcagcctggagaccaggatggcacccccaagtcctttcaaagtcayctgcaatggaaactctcttgcttttagtttttcccaggacagtcagccaa

SEQ ID NO: 418 - DNA, 101 bp, Homo sapiens
caaataacccacactttccttacaaatatgaattgacatatttatcacccrtcggtctggttttaggttttctattctgcgttgttctctgcctgactatt

SEQ ID NO: 419 - DNA, 101 bp, Homo sapiens
gaagtatggagacaaaaagttaaggagggtgagaggatagaggagtctcaytgaagatcccctggttaaaaccactgcctcatttctgtgaacagcctact

SEQ ID NO: 420 - DNA, 101 bp, Homo sapiens
ccatgtccctgtgtcatttttactcttggtgcttgtcgcctttcaacataytatatatctcatttgttttccttgtgtattaaccatttcccacattaaaa

SEQ ID NO: 421 - DNA, 101 bp, Homo sapiens
cacacagctgcaattgagtcctccactgatgctaccaggagctctagaackgggatggggccttcagggtgttctgaatttgggcaaggaggctgggcttt

SEQ ID NO: 422 - DNA, 101 bp, Homo sapiens
aactcagagtggatttggccatgaaagataaagtaaaagcaagtataacaygaaagaacaaaaaagcatgactcatatctgtgcaggctttttaatatgtt

SEQ ID NO: 423 - DNA, 101 bp, Homo sapiens
gccctataagagaggacagcagaaacaacagaggaaaaagtgacagggtckgctgttgaaatgcttatcaaagagtgggcatttgaactaagttatgaaag

SEQ ID NO: 424 - DNA, 101 bp, Homo sapiens
gctatcataaaacaaatattaagcacagcccctaaataatctttggcagtrtatgtcttggcaattttgatgtaattatgtttcatcattttctactttcc

SEQ ID NO: 425 - DNA, 101 bp, Homo sapiens
acttacactgaatgcaatacatagtaatttgaacaggagtttaatctagtyaatggggaccctatggagggtcagaggactccaatagccagtgtgagttg

SEQ ID NO: 426 - DNA, 101 bp, Homo sapiens
tagaaaaagaaagtaaaaaaggaaaattcatgaactgaaaaaagagtgacrttttcataaaatgagagaaaataaggtctatttataggtggaagggctga

SEQ ID NO: 427 - DNA, 101 bp, Homo sapiens
atgaataatattcccttctgtatatgcaccacatcttaaaaaattcatttrtctgtagttagacaagtaggttgattccaaatcttgactattgtgaacag

SEQ ID NO: 428 - DNA, 101 bp, Homo sapiens
aaggagataatagtgggtgggtgattacttgaaactgatttttggagaagktcattaattaaatattcattcattaattaaagaaacaatgtatgtcaata

SEQ ID NO: 429 - DNA, 101 bp, Homo sapiens
ttctaacccagaagctttctatttttttgttttcagaagatccccagatarcatctatccaaactaaaatgagaacacagtctgacggacatgaggggatt

SEQ ID NO: 430 - DNA, 101 bp, Homo sapiens
actcgtggagagtgcttctgcattttgatactctgaagtgattcctgcaarcaacagttgtttcacattctagactagaacttcagagtcatgtacaactg

SEQ ID NO: 431 - DNA, 101 bp, Homo sapiens
gcttggtgatactctttcaagccttgaaggggcctgttgatctttccctaytccactgccaacttcagttctccagttctctaaagtggggctttattcta

SEQ ID NO: 432 - DNA, 101 bp, Homo sapiens
gttcaagagttgggcatcttaactactttatcctctgctgtcaaagttctyaaaggtctcttggtctctgatctgctgccagcctctgcctggctggtaaa

SEQ ID NO: 433 - DNA, 101 bp, Homo sapiens
aggactggacatatctgcactcctgccctctgacttcagccgctacttccratatgaggggtctctgactacaccgccctgtgcccagggtgtcatctgga

SEQ ID NO: 434 - DNA, 101 bp, Homo sapiens
gggtctggaaggacctctgcctgggtgtttgacttggaaggggacagtggytctgggcttgggttggaattcagaacccatccccgggcagctgcgtgggc

SEQ ID NO: 435 - DNA, 101 bp, Homo sapiens
gtctttacagaactagagttcagggggaatatcagaggtaaaaaagctgaraaaagcattgacttcaaatgccagataccattttgatttttggcagagca

SEQ ID NO: 436 - DNA, 101 bp, Homo sapiens
tggaggtgtgggatagccagtattacaaccaagagtttacatctgtgttcyccaggcccacttaaatagaaccacagctaccaatcactgccatttatcat

SEQ ID NO: 437 - DNA, 101 bp, Homo sapiens
atccagtgtctgggggtgggaacgagagttatcatatggccaaataacttmaagctgagcgatgggcatgtggcatttattgtacaattctctgtgctttc

SEQ ID NO: 438 - DNA, 101 bp, Homo sapiens
ctttctaaatggaccctaagcttctctaggtcaagaaccatgatttagggktcttcgatgtgcctatcacttgagtcaaaaaccttaaaatagtaatgggc

SEQ ID NO: 439 - DNA, 101 bp, Homo sapiens
tgagattacaacctagtagaagcctgtaagtcagtgtctacatgacagcaytttgcatgccaagtccaggccatgactgctcattgtagacgttgcttgtg

SEQ ID NO: 440 - DNA, 101 bp, Homo sapiens
ctgtatagtttgtgagttattgcaaagggaggattgcccaggaaccatacraggctgctgtggagcagactcagccagtgctctcatatccatggtctccc

SEQ ID NO: 441 - DNA, 101 bp, Homo sapiens
tagttatgaagttttaggggaaatatgtccccctttttcacttggtaccamgttttgagataggcaattttctttgtagtcccctgaggaaggatttgggg

SEQ ID NO: 442 - DNA, 101 bp, Homo sapiens
gttacaagtcagccgtctgggtgttaaatctacacgtaccaaataaccaaytgtacttttttcactgaaatgttagtattatgtagagacagccacgactc

SEQ ID NO: 443 - DNA, 101 bp, Homo sapiens
ttctctcttacatgaacaattgaacatttgttagacatagtgatgctcctyagtattacccattcacttttttgggaggcacaagaaaggattgcacttca

SEQ ID NO: 444 - DNA, 101 bp, Homo sapiens
ttgaatccagaagctggccagctgttccaaatcagctattgttatcaatckcctctgaaaatcaacttatcaagcagttcacagctatcagatgttaaaaa

SEQ ID NO: 445 - DNA, 101 bp, Homo sapiens
cctgctaattctttctccatctgaggggtgagaaagacttctttttagctrtctctttcactgccaacctgctttgataatgttctgggggctttaccaga

SEQ ID NO: 446 - DNA, 101 bp, Homo sapiens
aaggcccttgagactgaggtctcaacagattgggacaaagaaggcaacagrataagggcataggtgttaccctgggaccccagagacctgaattctggctc

SEQ ID NO: 447 - DNA, 101 bp, Homo sapiens
ccagggtttcagacaagtctagagcaagtcaggatatcaataagacccaayaggatgtagggctgcctgtctagggagacatttagcttatcttccccggc

SEQ ID NO: 448 - DNA, 101 bp, Homo sapiens
ctcagctggagagcaaccctttcggtttaaaataaactaatgaaatccctraggacaaatatcactatgatatgcacaaaaacagcacattaatgcaacaa

SEQ ID NO: 449 - DNA, 101 bp, Homo sapiens
ttttctcttaaaagactcagtacattattagaaatgcctttcactaacatytaacaaataaaacagttctatagggacaatgaagttgacatttccattgt

SEQ ID NO: 450 - DNA, 101 bp, Homo sapiens
tctactggtcccatgtcccagagatcacaatgccttcctatctatcactgycggccattgctggtatttaagggtatatctctcttctgcctccaccctag

SEQ ID NO: 451 - DNA, 101 bp, Homo sapiens
acagtcttcagtttatttctcactgaactgatcctttgtttccctcccccyaccacctacagaatctaaattagagtgatttcctcccgcagaaaagtcag

SEQ ID NO: 452 - DNA, 101 bp, Homo sapiens
gcatctttaggacttctcccttgggattatcttcactattagcttttctcrttttgttttattttttcacatcccctcaatggaaggcaatacacttagca

SEQ ID NO: 453 - DNA, 101 bp, Homo sapiens
ttctaatcattcagataaaggtttaatttgtaccaagattatcctcaaaayatcactgaatacagtaaacactggcaattgccattaaaaacaaattatat

SEQ ID NO: 454 - DNA, 101 bp, Homo sapiens
tcatgttcctaaaaggacaacatgaagtataaacccaaacaatagatgtamactaatcatccctaacaatatccatagtgaatggttccaacagagtgcac

SEQ ID NO: 455 - DNA, 101 bp, Homo sapiens
catgtactagcatcaagaaacatctgactcccattctgtcattctgtaccyacgtcatcttgactagacatcaattaagagtttcctggaaaactcggaac

SEQ ID NO: 456 - DNA, 101 bp, Homo sapiens
gaccagactaaccctttttccttcttttggaggttatgattaggattgtcmgagggcaaagggtttaattttttcattaaactaacaacatgttttgagca

SEQ ID NO: 457 - DNA, 101 bp, Homo sapiens
atctcctagcctacaaaattattctttagagaatccattttcccacaagayatgcaaaaactaaaacaaaccacaacacgtgggccagatgtttcttcaat

SEQ ID NO: 458 - DNA, 101 bp, Homo sapiens
gaagaacgagccgtttaaatcacacatcagaccataccattcctctgctcraaaccctgcaatggtttcctgtttcactcagggtaaaagctaaaggtcct

SEQ ID NO: 459 - DNA, 101 bp, Homo sapiens
ggaatttttagagaaactacatgttctaacatgttctcttagggtgcttcrtacagatcgtcaaggaagtatcccaaaaaaaatcaatgaacacccggaat

SEQ ID NO: 460 - DNA, 101 bp, Homo sapiens
ttttgtccccattttttctcccatgtaagacatttttaatctaccttgcartgaagaggctgttaaacacttgtaccagcaccacccagcttttccatgtc

SEQ ID NO: 461 - DNA, 101 bp, Homo sapiens
ctggaagttaggatttgtacaaaagattgaattagttctcagtgacccctygacctaacccttggtccctcactgagtgggctccttggagcgctgtgatc

SEQ ID NO: 462 - DNA, 101 bp, Homo sapiens
ttgaaacattgtttttgtagaatttgcaagtggattttacagcgctttgargattttagagcaaatgaataaatgtaatccaccatataaagagaaccaaa

SEQ ID NO: 463 - DNA, 101 bp, Homo sapiens
ttttaagcaataagcatgctgtgcttaggctgtctcagcactattgttaartgctttaattatgtaacttttgatacattcatgttatcatatgttgtaat

SEQ ID NO: 464 - DNA, 101 bp, Homo sapiens
ctgaggcagtgcatacccaagactgtcacttctgctctgcatacctttaakattcttccttaggattctctagtacacagtggtctcatccaccagctgcc

SEQ ID NO: 465 - DNA, 101 bp, Homo sapiens
caaggagaggagataagcatcctcactacaacctgaccaattcttaaccayagaatctgtaaataaaacaaaatggttgtttgcctctgagtctggggatg

SEQ ID NO: 466 - DNA, 101 bp, Homo sapiens
atgtcaaaatattgcaaagctcctactgcaaatggctcatgtaaccaacaytattagagaatatttcctgtttagaaatttattttaaaaattgaaattaa

SEQ ID NO: 467 - DNA, 101 bp, Homo sapiens
cagaggtgtcacttgttttaaaagtgagaaactaaccagtgcttagaactrtaacccccagagcattgcctatgaataccaaggacctagaaatctcctca

SEQ ID NO: 468 - DNA, 101 bp, Homo sapiens
ggctgaacagatgaaattgctttagctaaaggaagtggcacgaatttactyatttattagatgtgcaggatacatccatcacaccgacctctggatcaact

SEQ ID NO: 469 - DNA, 101 bp, Homo sapiens
ttcctcataaacatcaagtaatgtgctggtaactgggaaatactgcagttkgttagtagaattttatcagaagtcaacaaaatattccgttttgcatgcct

SEQ ID NO: 470 - DNA, 101 bp, Homo sapiens
cacatcatctggaaataaagaacattttgcttcttcctttcaaagctacaygctgatctatcttgaagtttatgggtgtgggttcttctgccatctcaaat

SEQ ID NO: 471 - DNA, 101 bp, Homo sapiens
gcagtatctcctgggtatgtccatctggttatgtaaagtgaattattggtrgctttccccagctctttcaatttttaaaaaataagtaatacatccaatgc

SEQ ID NO: 472 - DNA, 101 bp, Homo sapiens
caggtgatagattaaaaactatggttacttaaaaaatgaccattgaacttyataaaactattctgcctgatttccaactggtatcaaaattttaagtgatc

SEQ ID NO: 473 - DNA, 101 bp, Homo sapiens
caaggataattatggctatcttttgtgtcttaattttgtttgtagtttcaygtgaaagtcttcattctggggggcttagaattaaagccctctttatttag

SEQ ID NO: 474 - DNA, 101 bp, Homo sapiens
tgccaagcataatcttaccatagggcctttgaacgggctatgcctccaccmgaaccacttttcccgtttatctgatcactccttcaccttcaagtcttgat

SEQ ID NO: 475 - DNA, 101 bp, Homo sapiens
ttgtatatactggaatagagtaaaccatacaacaaaacagaactctgtctrtatcaggaaaccttgtttaattttagggaaaatgatatacatttgaatac

SEQ ID NO: 476 - DNA, 101 bp, Homo sapiens
tatcaaaattttaagtgatcaagagtaaaagaactttatcaagaattatarcacttaacaggtcgacacagatgcagcccttttattatataggtataatg

SEQ ID NO: 477 - DNA, 101 bp, Homo sapiens
taaaatgttgggtggagatggtgccttttccagtggaagctactcatggcrtcagaacaaacccaccccacggacaaattcacaaagggtgtaaaactgga

SEQ ID NO: 478 - DNA, 101 bp, Homo sapiens
tgtcatacattggcccagcacatatgtgtgattgtgactctaatatacacrctcaactaaaagttaaaggtgtcaccctcaaagatcaggagattgtgtca

SEQ ID NO: 479 - DNA, 101 bp, Homo sapiens
cgaagaacagagggccaggaagctaattaataaatgacttgctcaagacarcacagctagcaaaggcagcctgatgtggagcacagcccagcctcttccct

SEQ ID NO: 480 - DNA, 101 bp, Homo sapiens
tggttaatttctactattacagtggtccatagactcatttgaagcaaattyatgaaaggaatattgccgtaaattcgatgggatttcatcaatatcttaaa

SEQ ID NO: 481 - DNA, 101 bp, Homo sapiens
agtttaaatgcctacagcaatcttccaagacacaggtgctatttttgatarcactatggaactgtacaaaactatacaaacaacattatgactctgcactt

SEQ ID NO: 482 - DNA, 101 bp, Homo sapiens
gccagtactgatggccctgtgccttcagtctagcgtcctggagtctgaaaygggagatggaagacagtagcttgaatacagagggtgaaagattttcctcc

SEQ ID NO: 483 - DNA, 101 bp, Homo sapiens
acaagcccagagaaaacatccatacaacaggcttgaaagactccaagaatmtctcgcctaaaaaattggtatcatatttccccagacaaaagccaacttaa

SEQ ID NO: 484 - DNA, 101 bp, Homo sapiens
aaagatacagggagtggactgggctttggaacaactcagttttacttccayggtattctgatgctcaagcagccacagaactcagatttcagggcagatga

SEQ ID NO: 485 - DNA, 101 bp, Homo sapiens
ttgagttcagtgtgaggaggtttatgcctagaaaaggtgctcaccaataaygtgcctcagttcccataatagcaagatcgagaaggttctttagtctcccg

SEQ ID NO: 486 - DNA, 101 bp, Homo sapiens
aaaacttcatacctctccagggagacagttcccagaaacctccctcccctrcaaagcactcctataacaaataaataaactacatttcccaaagttctctt

SEQ ID NO: 487 - DNA, 101 bp, Homo sapiens
ttctccttcaggaattcttatcgtgcataagttagttctctagatagggtyccataatcccataggccttctccattttttttcactcctctgactagaaa

SEQ ID NO: 488 - DNA, 101 bp, Homo sapiens
atccctaactggagatcatctcctcagtgctggacttgagattcaaattcrggaccttacttctgagtctgctcaaaagcactctgaaacagcatccagag

SEQ ID NO: 489 - DNA, 101 bp, Homo sapiens
tttattctgtaatgtgattataagccattagcaggatttatgcaagggagygatatggtagatttacacgcttaagagattattttgcctgttgggtagag

SEQ ID NO: 490 - DNA, 101 bp, Homo sapiens
gatactgatctataaaatataagccaaatactgttaagaaaagttaaccaygaataagccaggtatggtggctcatgcctgtaatcccagcactttgggag

SEQ ID NO: 491 - DNA, 101 bp, Homo sapiens
atctggaagacccaccctcaagtggtacataccagtgccattcacattctrctgcctaaattactcactttgcctcacccaactttcacaaagcatggcaa

SEQ ID NO: 492 - DNA, 101 bp, Homo sapiens
tgtgtcatttaaccttgcagaagtttaaattctaccagtatttcctgttayagtttctgcctttggtgtcatgtgaaaaaaaaagaccattactatagcaa

SEQ ID NO: 493 - DNA, 101 bp, Homo sapiens
tattccattaactaaacagcaacctcgaaagaaatcaatactcggaaggtyctgtagtagcagccattccatggatgggacaccagaggtggggcaggagc

SEQ ID NO: 494 - DNA, 101 bp, Homo sapiens
gctcccagcagctcacccctccagtggctgttctttctacctgtcaaagcytgtgctgacacatatactgggaggtgacccccagctgcggctgccccacc

SEQ ID NO: 495 - DNA, 101 bp, Homo sapiens
tcaatatggaagaacttgtccaggcttgtgcagaccaccatgtctctgccktacaggctgacatttaacaatggtgaaggcaatctcttcttggaaaaaat

SEQ ID NO: 496 - DNA, 101 bp, Homo sapiens
agactgtgcagtgtccagttcttttattaagtacatggggtctgtagtcayacttcctggggcaaaatcctgcctcttatgtttttgaccttcggcaagtt

SEQ ID NO: 497 - DNA, 101 bp, Homo sapiens
gtttagcatctgtggtaagtgtgttcgaaggccgtgtaagcacattttatyatgagcatgtcttacttccaagttaagataaagatttggaaattaatgta

SEQ ID NO: 498 - DNA, 101 bp, Homo sapiens
atccagaatctacctacattcattgttattaatttgtacccctggtgttcrgccagtatcaccttctcccaatctatttcagccagtgacaatgaggacat

SEQ ID NO: 499 - DNA, 101 bp, Homo sapiens
agagatgcccccgccctccagggaaactgcacagacattacaaacaagcaygctcttatcaagcaggagaggtctgggtcggggggctggggggaaggatt

SEQ ID NO: 500 - DNA, 101 bp, Homo sapiens
ttattgctgaattggtataaagatgaatatatgcctggctgcattctactyattcttcttatttcaagagaaattaaatcatttcatgggcccctaaaatt

SEQ ID NO: 501 - DNA, 101 bp, Homo sapiens
ggctaatcaatttgatgtcttttaaaactaatattcttcaaattttttttyagtgtctatttaggggaatggctgatggctgcatgaagtgggggactcag

SEQ ID NO: 502 - DNA, 101 bp, Homo sapiens
tcttgcttccaggggaagctgccaggtagaagtagtgaggaatctggtatygcactgtcccaaggggcgggacacctgcctttgaagacccctgggttctg

SEQ ID NO: 503 - DNA, 101 bp, Homo sapiens
ctactgatctttcagactgcactgttcattctaattcttataatacaaagkcagagcagcagatactctagggaaagaatgcttgcaccgtgaaatccaca

SEQ ID NO: 504 - DNA, 101 bp, Homo sapiens
aacctccctccctgctgctatcttatgtacactcttaatgtgcctaacctyccacgagtgtgcagagatgctgctagagcagtccctgcttagatcactgg

SEQ ID NO: 505 - DNA, 101 bp, Homo sapiens
ttgttcaaaatgtatattttctcgtttttaaattatgtaattttggctggrcgtggtggtttacgcctgtaattccagcattttgggaggccgaggcgggt

SEQ ID NO: 506 - DNA, 101 bp, Homo sapiens
attcacacctcaggtcttcactttggggagcgaagccttttagcagaaatrccagaagtaccatcttgccaaatggtcaggaactgtctgatagagatgga

SEQ ID NO: 507 - DNA, 101 bp, Homo sapiens
aggcactgttttatcatggctcatctagattccaaagtccacaataacccrgatgatccatgtggtcatatcatgctcttcacaagtacatgcctctgctt

SEQ ID NO: 508 - DNA, 101 bp, Homo sapiens
tcaactacaggtgtgttcctgatggcctttagctggagcgtactgacacartaacaggctttgaaattcaagtgattcagtttggcatcttagctccacca

SEQ ID NO: 509 - DNA, 101 bp, Homo sapiens
catgaatatgtacaatgattatttgccaatcaaaattctgcatcctccagmagcatgctatccaaacttctttcatcatccctctccctctggaggaggac

SEQ ID NO: 510 - DNA, 101 bp, Homo sapiens
ttaacaaaaaacgaatattataaattgattatgtttccttgcagctggatrgcttagcctgaagtatggattgctagtaattcctccagtcactcaacatt

SEQ ID NO: 511 - DNA, 101 bp, Homo sapiens
ttcatccttaataaaaagaaaattgcatagctttttatattgttgcaaatkcatctcccaatatcattgtcagcttagtgatattctccatattttaaaat

SEQ ID NO: 512 - DNA, 101 bp, Homo sapiens
aatgaagtaaagcaagtttcagctgtttctttccccaatgcacaaccttartttccttttatcttaaacaccaggaatcaaacaatctcaaccatctgaaa

SEQ ID NO: 513 - DNA, 101 bp, Homo sapiens
tagtgtagccatccaatggactactatctagcaatgaaaaagagtgcaccrttggccacatggcaacagggataaatctcaggagtgttagagcaggtgaa

SEQ ID NO: 514 - DNA, 101 bp, Homo sapiens
cctttcctcttccccgcaccaacaccagctccatgtgcatttattgttggrttttaacacccgtgtcctccctccctctccccagtgttctttcacagctt

SEQ ID NO: 515 - DNA, 101 bp, Homo sapiens
gaagagaccaattgccctttttacagatattatgattgccaacacttaacrtgtaaacaaattattagaacaacattgttcagcaagattaccgagtgcaa

SEQ ID NO: 516 - DNA, 101 bp, Homo sapiens
ttgtatttttgacgtcactagtgtcattttttgagtcctctaccaattttycaagggtatatcatcttcagttccaattgaacatacagccctttttgaat

SEQ ID NO: 517 - DNA, 101 bp, Homo sapiens
aaaaaaaagaatacattttgtttagatgtggaaaatgagtagcttgaaagyaaagccaaacaacaacaaaaacaatgacaaaaaatctgtatgtcgtaatc

SEQ ID NO: 518 - DNA, 101 bp, Homo sapiens
cattttaatacgtgtcacactgaataaatttatgcacatttattcatgtcraaaggaaaaattaatgttgtgatgttgactccttgatgaagtttttgaag

SEQ ID NO: 519 - DNA, 101 bp, Homo sapiens
ttacagatttttaccatagatatactcatagagaaggtagcacccactcarccctagcaatagtgctagtgtttacaaaattgcaaaagaagtatgacaca

SEQ ID NO: 520 - DNA, 101 bp, Homo sapiens
tcaggcggcccagagagcagcgctccacattcagcttcacggagccagacrcaaggtctcgaatggtgaagctctggtctgcagccaccgtggttacctgg

SEQ ID NO: 521 - DNA, 101 bp, Homo sapiens
cccaaagaatccttcccttacagcaggccagaaagctattgtcctagcctrtggaaacacctaaaacacactggggagatgtggacactcagcccattgca

SEQ ID NO: 522 - DNA, 101 bp, Homo sapiens
caccactgacactatttacagccaaagaaatcatatgaaaccgtactagcrcatgcaccagaaccaaatccaaagtgccccaccaaaacaacaccataaat

SEQ ID NO: 523 - DNA, 101 bp, Homo sapiens
aattctcattctcctaatccaaggctctgtgtgatacatcacactgtgttyattactttattacagagcaagtaaacagatgcttagtgtagatcacgcag

SEQ ID NO: 524 - DNA, 101 bp, Homo sapiens
ctggattttgtggtcttatgctatttccactcattctccaaatgtaaccgkaaagaccatcccaaaatgtaatacaaacctttttaaatgcccatttaaaa

SEQ ID NO: 525 - DNA, 101 bp, Homo sapiens
caggagcagggtggacgtcaaaaaataatcctgatgctatttggctcatgkatgattcagagcaggtgctgtcagagccctaatttcccttgtttttgaac

SEQ ID NO: 526 - DNA, 101 bp, Homo sapiens
agcaaactgataagtcaaagatgcatatgtaattcccagatcaaatactaraaacagcaaaaagaggataaaatagccttttcagcaaatggttctagaaa

SEQ ID NO: 527 - DNA, 101 bp, Homo sapiens
tctattcaattttgtttcttttttcaaagtaaacactgttttgtaaataayacagaactgaaccccaaaatacataactgggcattggaggattagaacat

SEQ ID NO: 528 - DNA, 101 bp, Homo sapiens
agaggaaatgtcacaaaactcttcatagttacaaagacattgtgacactcrgtagaggtaaaggttccagtattttaaaaacatgagaaatatgggttaaa

SEQ ID NO: 529 - DNA, 101 bp, Homo sapiens
cttgctgctttctgcagaaacccctggaagcagtccaaatgcaaagttagrgcttcagagaatgcaccctgtaaatggtagttgtgtatacccttaccatt

SEQ ID NO: 530 - DNA, 101 bp, Homo sapiens
ttcaaggaaacaggtgaacatataaacgatgtaacagtttatatgtaggartgccctttggctctgtctattgctgtcagtacattatttacctgctccag

SEQ ID NO: 531 - DNA, 101 bp, Homo sapiens
ttttattagcaggtctagattgagagagatttacctcggcagtaccatagygtggataatattcagttaggtttgttcagaggaacttccccatcattctg

SEQ ID NO: 532 - DNA, 101 bp, Homo sapiens
acatgaatagatgggacctgtatttgcttaattccagtagactaaatactytggcctaaatagagttgtcaatctcataaacccaagaaatactcagaaac

SEQ ID NO: 533 - DNA, 101 bp, Homo sapiens
agagctgacttttacccaaggggctgtgggtggaaaccagatgaaatgggrtatgtagttgatggtatgtgaggacctaatactgtcttataaacatttat

SEQ ID NO: 534 - DNA, 101 bp, Homo sapiens
atcgccgttcccgaggtcgtcccctttgcacctgtccgcgggtcctcgggygtgtggcttccgggcacacagaaaaccgtgtggttctaggatacatgggg

SEQ ID NO: 535 - DNA, 101 bp, Homo sapiens
attagtcatggaaaaggaataaaaggcatccaagttggaaaagaagaaatraaattatctctgcttaaagatggtatgatctatatgtagaaaatcctaaa

SEQ ID NO: 536 - DNA, 101 bp, Homo sapiens
tctttctaatacacatattgcatctattccatgccttcaatgaatttcccrttgtttaaactataggtcaagaaactgtccaattgctatacttgtttggg

SEQ ID NO: 537 - DNA, 101 bp, Homo sapiens
taaagaaaaaaatctgtacatgttttggacagacacaatttttttcccagrctatttttgacctatagttatttgaatccacagatgcagaacccacggat

SEQ ID NO: 538 - DNA, 101 bp, Homo sapiens
gccttgtgtgagcatttttatctctggcaagctcccttcctctcttagatratagagattatctcccgggattacaaggacagcttctccacaacgaatcc

SEQ ID NO: 539 - DNA, 101 bp, Homo sapiens
cctagaccagtgtggcgtataggctatagaagattggtcctgaatgctaayggcagggatgagtgtaagatcatcaaaacttatttgtgggaaggagagct

SEQ ID NO: 540 - DNA, 101 bp, Homo sapiens
gggaaggtcgttgttttcctcctatttcaaggtgttgcacctttggccaamgggcccacagcactgcttggaggaaccacagggcttcaggacgtgaccgt

SEQ ID NO: 541 - DNA, 101 bp, Homo sapiens
ccaaaagagaaaaaattctgacgggggcataactggagaataaagtgatcytaaaatactgctgaaacaaaaagtcatctgccccctggaccgttgtctta

SEQ ID NO: 542 - DNA, 101 bp, Homo sapiens
aaatcttggctaatcatttaatctttgggcatcaatttcttcactgttaamatgacagttgtagtatttctccttaaaatacttcagggcagaattaaatc

SEQ ID NO: 543 - DNA, 101 bp, Homo sapiens
tacatgatataagaaaataataagaatgtggtttcgtttaggaagattctyaatacacaaagatatatctgcaaatatattttcctagctttggttttctt

SEQ ID NO: 544 - DNA, 101 bp, Homo sapiens
ccaagtaactataagattcatgtattagagaaaatcatattaaatttgctrttatgtgatcctttagacatataaaaatggtatatgttatggttcaacct

SEQ ID NO: 545 - DNA, 101 bp, Homo sapiens
acaattatatgccaacaaattggataccctagaaaaatgaaaaaaatcctrgatacaacctaccaagaatgaatagtaaaaaaaaaattcttactcaacat

SEQ ID NO: 546 - DNA, 101 bp, Homo sapiens
cagcagatacccttaattcctatttcccagtgagaacaaagggcagaaaaygtgaccgtgcccacattctctgctccctaaccccctaaacaatcagcacc

SEQ ID NO: 547 - DNA, 101 bp, Homo sapiens
atagcagcccttagcccagcgacctccagaagcctcgcccacccccggatrgtatacccaccctagagagtacgagtcctggcatttgaggaagtaccact

SEQ ID NO: 548 - DNA, 101 bp, Homo sapiens
taaactgttcagtaataacattgatttgattttaagaaataatagaaaaayagagtttatactacagcagtgatttccagtagaaatatactgggagccac

SEQ ID NO: 549 - DNA, 101 bp, Homo sapiens
agctagtgtccagtagtcctcccaggattataggtgaaagatggaggagamggttcggtatgcagggaatcacgcgacacagtgtccaattaatttttgtt

SEQ ID NO: 550 - DNA, 101 bp, Homo sapiens
tatgtagcagcaatcttaaaaaatttttatttactaaaaatctcatcatcyaataattatttaaataccttttcatactatctgtataagttagctaatgt

SEQ ID NO: 551 - DNA, 101 bp, Homo sapiens
ttatcccttatagatgcctaagagcttatttataaaatggtaatactaatrtatttaatgtcatcttacagttaccatgtacttttcagtttacaaaatac

SEQ ID NO: 552 - DNA, 101 bp, Homo sapiens
attttttacctgcaacccctgatgtggacattctcagaaaaagccagccaraggaagtctttcattaatcccaggcatgtcacataacctcagaccttttt

SEQ ID NO: 553 - DNA, 101 bp, Homo sapiens
tgagctccaagcaggcaaggaattcacctgaaagcatgaatgaaagacagrtctggaatgcaccaaatgactaggatcaggagtgtctgtaagtgtcagaa

SEQ ID NO: 554 - DNA, 101 bp, Homo sapiens
catgcctggacttcacttgtagcacatcatttgtggaaggctgcagtaagyactcaatactttgctgttgattgatttcagaacggattgatcagattgca

SEQ ID NO: 555 - DNA, 101 bp, Homo sapiens
tcatggaaatataaatggaattttagattcatgttaaacctctcttgtaamgttctcaatgtctatgtgtatacttcaaactgtaactttttttaaaaaaa

SEQ ID NO: 556 - DNA, 101 bp, Homo sapiens
ttcatcacaccactctgacttgctacaatgactgcctggacatgctgactmcagtgagttccaggcatcagtagggtctgaaaatataagcaaaggaaaac

SEQ ID NO: 557 - DNA, 101 bp, Homo sapiens
atctgtgccctcttagaatgtaacactggaaagtggtctccctcttatggyttttaaaattgtgaatgtgctggtttgagcaataaactctgaaggttgag

SEQ ID NO: 558 - DNA, 101 bp, Homo sapiens
atggatcactgcccagcagatagctggtcctccaaacgtatttgctgaatraatcaactgccttagaggcagagatattcttactgcattccttagtctat

SEQ ID NO: 559 - DNA, 101 bp, Homo sapiens
ttctgttacctaggagatgttacttacatatgtaatactgtatcctgcacrtggaaatattcagaattgtagatagcataactctccctgctcctattctt

SEQ ID NO: 560 - DNA, 101 bp, Homo sapiens
aaggcagcttgaccacaggcaatagcttgctgattcctgcataaagtttarcatactcttgaaatttcatttgtctaatattttaacctcaaactgtgcct

SEQ ID NO: 561 - DNA, 101 bp, Homo sapiens
tacagaaagccctctgtccttgtaacaaggtagacgctctaattgagttgrttaacacaaggtgcccgtaggcaaactaagagaacaccctgtaacacacg

SEQ ID NO: 562 - DNA, 101 bp, Homo sapiens
ttgtgcaaatcttctgatttgtgcaaagtcccagaagaaatgacgatagamtgctgctctcctcctaagtaaaatgaagaagtatctaagagaaacagatg

SEQ ID NO: 563 - DNA, 101 bp, Homo sapiens
cagataacccttaaagtgaagaactaggtgtctcaggtagttttaggtacytcacctgcttcctgtaatctctacagacatttgcttaaatatatactaat

SEQ ID NO: 564 - DNA, 101 bp, Homo sapiens
gacctcaggtgatgtttagacttacttcttggcctagacttatgttaacaraaccccaaaaggtctaaagcactaaagaggtttgccaactacacttagat

SEQ ID NO: 565 - DNA, 101 bp, Homo sapiens
tattttagtaccaaatgaaatttccattcagatataatttgcgaacccctygggtgacacttccatgcaatgaaataatactataatgacacaatgacaga

SEQ ID NO: 566 - DNA, 101 bp, Homo sapiens
tcactcagctaatagacagagaatgatgtataaaatcataatgccaacttrtaaatttataaatagaaatatggttgtcatacctccttaaacactgacat

SEQ ID NO: 567 - DNA, 101 bp, Homo sapiens
aagctggctgaatttttacaaggcaggaatgaaatactgaagagagacatmttcttgaaccaaaacaagctgaagaagagtattgtcccaaatattgcaca

SEQ ID NO: 568 - DNA, 101 bp, Homo sapiens
ggaatatactgtctctcagtaagtgatactgggacatctggatatgcatayagggggggaaaaaaagaaacgactcctacattacatcgtacacaaaaatc

SEQ ID NO: 569 - DNA, 101 bp, Homo sapiens
tcaatttctgttcctttaggccagtcagtctgtgttaccttcttacagcgrccccaggaaacgaacaagaaaccagtccaaactgcttagcatgatactta

SEQ ID NO: 570 - DNA, 101 bp, Homo sapiens
tgtggatgcagaacccatagatagagagggctgactgtactaaagattacmtttccttctccacgagtctcaacatattcatctactcagcagtaaataaa

SEQ ID NO: 571 - DNA, 101 bp, Homo sapiens
ggaaaagaaaagaaatggcaacctgaggtcagctgtgtgtgacccacatgyaagactgaagtagaacttgcctccttgtgaacgaaacagggcaacaagag

SEQ ID NO: 572 - DNA, 101 bp, Homo sapiens
catcactctgctccatctcttacctagattccagaactcttctttctccayctacccaaacttttacttctgctagtctctattacccatgcctttctaca

SEQ ID NO: 573 - DNA, 101 bp, Homo sapiens
atcctcaccactgcaagcattaaggagaaacccctaaaattattctgagtrtaaacacagcaaaaggcgcatggaccttaaccaacatgtatgacaccaaa

SEQ ID NO: 574 - DNA, 101 bp, Homo sapiens
tggcacaataactaactgtatttttagagtttatcaataaatatgatgttrccataaacacacatgaacacactgatctctttaaaagatttacaatggaa

SEQ ID NO: 575 - DNA, 101 bp, Homo sapiens
cccaaaggttgttatgaggtgcatgactttacttatttgtgtggattgatygttaatcagtatgccatattcctaaaaatgagcacttgctccaggtcttt

SEQ ID NO: 576 - DNA, 101 bp, Homo sapiens
cagtcagaacatctttggtacacccatggaatgaatagtaatgactggcaygggcagaggtgaagccatatccatatgtttattttttattaaaaaatgta

SEQ ID NO: 577 - DNA, 101 bp, Homo sapiens
tttcacaggctcctgggcccaagatacagtgagtacaatgggtcccacgcrgttctcccctttgagtttgaaagcttagagttcttgagctgaagcaagca

SEQ ID NO: 578 - DNA, 101 bp, Homo sapiens
gggaaggcacttgtttcgtggaggagtaggatttgtgtctctggcagttgycctgcacattcaagatgcaagagctttctgtgcaacacaagcaaagcaga

SEQ ID NO: 579 - DNA, 101 bp, Homo sapiens
caggtcatgttttcacaaaatgtgacatttcatgtcgttgttatgaaaacmgtggcaccaaattcaatctgcaccaatcatatttttattttaatatttta

SEQ ID NO: 580 - DNA, 101 bp, Homo sapiens
gatgggaactggcctccttttaatagcacattaacaacattattctacccraaggaagacagcttccctttggccttagctgccttgtgagtttggtgaac

SEQ ID NO: 581 - DNA, 101 bp, Homo sapiens
aaaattctgtcaatagacacataggtagggagactattcctgagtggtgcmtgcctctagaaaaacaaacctataagtgagataaagtttagatttcataa

SEQ ID NO: 582 - DNA, 101 bp, Homo sapiens
tacatatgcttcagaagaaggctaagggttcgttatcttaaagggggaaargagtgtcttggacaccagccttagctgtcagacaggtctcatcttaattc

SEQ ID NO: 583 - DNA, 101 bp, Homo sapiens
actattcccctcagtctcctcactatgcatcaaaactagcaggtaaatccytggctcatgatgcatccataagcttttctctcacttttctaaaatattag

SEQ ID NO: 584 - DNA, 101 bp, Homo sapiens
tctcaaacttctgctctaaactggcaacatttaaagagtctatttgggaaytttggggaacccagtactctcctattggtgaaaatgagagaggatgcagc

SEQ ID NO: 585 - DNA, 101 bp, Homo sapiens
aagtgtaatttacaagacagaaaggccaagatactcgaattgatttaacamgtacaggcaaagtatttttgaagaagttatttaacccatttgaaactgat

SEQ ID NO: 586 - DNA, 101 bp, Homo sapiens
tcccatgtttacacatatattcattatacattttatgtacctattatgatrtgccagtcaccttgttaggctttgggtataaaaagaatacaaagatgaaa

SEQ ID NO: 587 - DNA, 101 bp, Homo sapiens
gaattgcaaaaggcatttcaaagcaccttcccacattcccagaaagatgtyttcccctctttccaaacagctgagacagaagtacaacgtgtggtccctgc

SEQ ID NO: 588 - DNA, 101 bp, Homo sapiens
gtcatatatcaattatacttcaattaagttgtaaaaatagttataaaagcmaaaggtatgtctgcactgttttatatatattcattttaatttaaaatgtg

SEQ ID NO: 589 - DNA, 101 bp, Homo sapiens
cgtcatcccttaacagaactgctgcaacagcagtaactgatgttccatgcycccaccccttatagtgggttaccaacccagatgccagagttacgcttttc

SEQ ID NO: 590 - DNA, 101 bp, Homo sapiens
gaggatatggactgaagagtagtatttacacagtaaatgctaccagccagrggaagaagaggaagatgtgtgtgaacctgagcagtcccacagtcctgtcg

SEQ ID NO: 591 - DNA, 101 bp, Homo sapiens
gagagctgttaaagggtttggagcagaggagggacatgacccaaccagccyattaacaagagcacaggctgatgtgttaggactgaactggagaagacagg

SEQ ID NO: 592 - DNA, 101 bp, Homo sapiens
ttatttaatgttgctcttgtatccagcaaccttgctgaatttttttatttktaatagtttggagtagatactccagttttacaggtaaatcgtcattttca

SEQ ID NO: 593 - DNA, 101 bp, Homo sapiens
ggaaaagctgttaggaggtgctgaataataatcacagttgagtcactttcygacactgctgtcttgcatgatttactgaatataatccttcaaatgatctt

SEQ ID NO: 594 - DNA, 101 bp, Homo sapiens
tggccacatgtgcctgttgagcacttgaaatgtggctagtccaaattgagragttgtgctataagtgtataatacacactggacttcaaagacttatcttt

SEQ ID NO: 595 - DNA, 101 bp, Homo sapiens
tgcttcaatgctttctgatttcatacctgcataataaaattcctgattcgyccatcacattttggcaaacaaccaccgccacatctctctggatactggct

SEQ ID NO: 596 - DNA, 101 bp, Homo sapiens
gtagcttttggcaaatcttctactgcatctcaccactgtgggaaattgcarcttccaaggaaaaggagtagaaactacaggctcaaaaaaatgagatcagt

SEQ ID NO: 597 - DNA, 101 bp, Homo sapiens
aaaatagaaatgtattttatattctaaatcttaagagtcattaggttgatrtttgcaattttttatagttaatgcaaggcatgttaaaatataatttgtct

SEQ ID NO: 598 - DNA, 101 bp, Homo sapiens
ccggaatagaactcaggctaaatgctggtggtatggaattgggaacatgtrccaagtaaagacagaggcttgtttggaaggaatagcagaggaagatgaaa

SEQ ID NO: 599 - DNA, 101 bp, Homo sapiens
atgacgtccccatgacacagagaagccagaacccagcacgcaccccatggycattgcacttcttcccacagccttcagtttcaaagaaggaggtgttcctg

SEQ ID NO: 600 - DNA, 101 bp, Homo sapiens
cattaccagatattctgtagttctttatttctgaaattccttaattggaaracaaaacaatagtaatagccaaaataaaagttacatggatatagtttcat

SEQ ID NO: 601 - DNA, 101 bp, Homo sapiens
ggttaagaagcttaattgcaatccctatgaataacaaaagttgttagaacyacaacatatcattttcctttctctttagtagcagattgacaaaaactggg

SEQ ID NO: 602 - DNA, 101 bp, Homo sapiens
atgtatcctgtaggcagtaggtcgtgtggatggtttttaatgtaaaagtgyggcacgatgacagcattgctttataatgattattctggtggcattattca

SEQ ID NO: 603 - DNA, 101 bp, Homo sapiens
catggcaatgtggagaatgaattggaaaggaggtgtggaggtcacctagcrgttcaactgaggtaatataaaggtttgaaatcaagcagtgatgagcaaga

SEQ ID NO: 604 - DNA, 101 bp, Homo sapiens
cttgtatcaacttgttgtttatgctctctactaaatacatcctgtatgttycaatccttgtgtcttttcttctctcctttaatttaaatattacttcttgc

SEQ ID NO: 605 - DNA, 101 bp, Homo sapiens
tggcccacctgggatcttctaggtctttctatcacaatactgctttagaaragtctgtgtgaaggaggggactctggtatttaactccatccatcaatgtc

SEQ ID NO: 606 - DNA, 101 bp, Homo sapiens
attttgaattgtacaatacatcataattattggagatagtcactccactaygcaatagactccaaaggtattccatctgtttacctgaaactcttgggcca

SEQ ID NO: 607 - DNA, 101 bp, Homo sapiens
ggagaattttcccttgctccggcttcccactgacggacgtttcacttaacygtattaattcctctgcactattagttacgcatgatgcatgacaagcagat

SEQ ID NO: 608 - DNA, 101 bp, Homo sapiens
taaatccttcctactgaccagtgatgaagacagtgtccatttctagggtamattgtctgcgattgctgcactctgatacatgagaaatacatgggagggag

SEQ ID NO: 609 - DNA, 101 bp, Homo sapiens
aacagttttcttaagttactttttctgtccttttagtggcttcatttaaaktacagtaaaatctcagacacaaaattatcaaggatttaggaataaaggga

SEQ ID NO: 610 - DNA, 101 bp, Homo sapiens
attccagaaatggtaaaaggtagattcaaagtgtagcaggataaaaggaaragctatttcagggtctctgttaatgaggacatcaaccaaagttttcccag

SEQ ID NO: 611 - DNA, 101 bp, Homo sapiens
gcattccaggtagaaggcaagggtcagagtgccccttcctagtttctctcyatccatcattgggacaaaatcttccccagacgcctcagtatacttcccct

SEQ ID NO: 612 - DNA, 101 bp, Homo sapiens
ctcctttctttggctatttttgatatgcctcattttgtatcatataaaacygtggctcttcttctcttactgcatataactttaccttctactttatagaa

SEQ ID NO: 613 - DNA, 101 bp, Homo sapiens
gtcttcaggaggtaagaaatagtaggagcttcttgaattttggaaatcagracacaaaatagaggatacccctctgcagcagaattttaattcaacatcat

SEQ ID NO: 614 - DNA, 101 bp, Homo sapiens
agttatcactgacccattttctatgttatcctaagcatcctttgaacgatrtcctctaaactcttctcacatattgacttcaagctcaatagcctgtgatt

SEQ ID NO: 615 - DNA, 101 bp, Homo sapiens
ctctttgtacttttctctcccaaaggagcattccttgagaagccggaggarttctactgattacatctccagcacagccacattccagcgggtaggagggt

SEQ ID NO: 616 - DNA, 101 bp, Homo sapiens
gaagcagagataatgacagagagtgggatactagagaaacgcccaagaccrtctttagctgcagagttctatcctggatttcatgtgtgaccttagacaaa

SEQ ID NO: 617 - DNA, 101 bp, Homo sapiens
taagagggggcactgctggatttggtccatgttataggatttgctgcacakcccgttactcagaaaatggggctgtggtatcagacccggctttgaaactg

SEQ ID NO: 618 - DNA, 101 bp, Homo sapiens
agcatggttataatagaataagttaagttccaaataggattacttatttcrtgttgtagccctaattttgcctcaaccactcaccctctggtaaattcctc

SEQ ID NO: 619 - DNA, 101 bp, Homo sapiens
accatgagtaattcagtattcattcaacttgaataactacagggttaggakagtcattttgaaaatggttaggattattagttagtgttaagaaaatattt

SEQ ID NO: 620 - DNA, 101 bp, Homo sapiens
tgaaaagagaaatgcatatagattttttagatgaaagaggggagcacacarcatcccaaattgtgatatcgtttttgcctaagcaccaggggttttaggga

SEQ ID NO: 621 - DNA, 101 bp, Homo sapiens
gaaacccgagcgaaagacatttcaaagagggtttagatttaaagcaaatayctattcactctaatctgctttaaaatctgttgttttcctggagagactta

SEQ ID NO: 622 - DNA, 101 bp, Homo sapiens
gtgagaatgttgatttttgaaaaaatgatccctcaaatgcttacagccccrtgcatgtacaaagatgaaaaatcagtgcaattggagaaaaaaacaatggt

SEQ ID NO: 623 - DNA, 101 bp, Homo sapiens
taaaggattctaagtcacctttttccctcattcaaaatgaaaacctctctrtttttatttattttttgagacaaggtgtctatcacccatgctgcagtaca

SEQ ID NO: 624 - DNA, 101 bp, Homo sapiens
gacaatttccctgatataaaggaaagatgaatttgccaaatgagcagcaartaattttccagggtaaggtgatggagaatgagccacactgatacaaatcc

SEQ ID NO: 625 - DNA, 101 bp, Homo sapiens
caggctttaccacattaattcccagggtattttcctaaattaacatcaacmttacacttaccattgtttctttagtttctcaaaactttatcataatgtga

SEQ ID NO: 626 - DNA, 101 bp, Homo sapiens
taccattttgtgtctttacatcttttactcctggcaaaatgaaataatttmttgatgaatgtattagtttttgtcttttaataaatatgctgtaagtgttg

SEQ ID NO: 627 - DNA, 101 bp, Homo sapiens
tactcaccattatctctctatggaaataatctgcctattattgcctccctrtggaatctgcctctttatggaaataatccccaacataaagcagcaactcc

SEQ ID NO: 628 - DNA, 101 bp, Homo sapiens
ctcagtaagtggcactctcatgttttaaagttattcaggccgaaacttcaytctttctatgtctctcactgtgtaaccagtacattagataatcctactga

SEQ ID NO: 629 - DNA, 101 bp, Homo sapiens
ctaattgactgctgctgaagcaattaactgattatgttttcccctcatttraaagtttctgtgatatagacaagtaactttgtgttacaaaagtaatctag

SEQ ID NO: 630 - DNA, 101 bp, Homo sapiens
aagctggcttcctcagccatcttgattttgaatactttgccacttctgaayagtttagtgtttttctgttctatccatatggtgacatcagctcttagttc

SEQ ID NO: 631 - DNA, 101 bp, Homo sapiens
caagacttgctagacacaaggtccaagctgacatagatacctgggaggccraaagcagcaacactctcctgcttgggagaggatggtacttattaaatgga

SEQ ID NO: 632 - DNA, 101 bp, Homo sapiens
gctgatttaactatgtttctttttggagcaattatttttatgataagtaamagaaaagtttactcatacagagaaaaattcaagaaggtatgaggcactga

SEQ ID NO: 633 - DNA, 101 bp, Homo sapiens
atgattctgaatgtgattggatgagttcctaggaagatgggtcatacagayaaaatgatcattgtagagaagatgctatttcctcctgtgggaaagaaaca

SEQ ID NO: 634 - DNA, 101 bp, Homo sapiens
ggatgcatagagttattctatgtaaattacccaccagagagaattcaggcrtgtttcaaaatctataaaaccgtgtgctgggggaccatgaaaagttgtag

SEQ ID NO: 635 - DNA, 101 bp, Homo sapiens
aatttgataattaaaatttcattgatgtgtttgcacttattctcttaaaaytgtaacatttaataagtaaaaagttatgctcattaactcaaacagatttt

SEQ ID NO: 636 - DNA, 101 bp, Homo sapiens
ctcaggtaaattcacctatgtgtgtatggtaagacactgcttctactctgytcatcagcaaaacacttattatcattttcataactttcctagaattttag

SEQ ID NO: 637 - DNA, 101 bp, Homo sapiens
tctcagggtgaaattcagtacaacttcattttacagtaaggatcttggggyccgcaggagattttctgtgagaaaattgtaagagagggcccctgagaagg

SEQ ID NO: 638 - DNA, 101 bp, Homo sapiens
tcataatgggactgcagaaccagaagcaaaagagtaaaatgcttattttcrtacaacattgagttttggggtccttggtttgtaacattattgcagtaaaa

SEQ ID NO: 639 - DNA, 101 bp, Homo sapiens
aaaaccatatgccattgtatctgaaatgttggcccccttcaagactctcamccaagaaattgcaccataatttacctcattgttgaagccaagaaaatgga

SEQ ID NO: 640 - DNA, 101 bp, Homo sapiens
gcatcatcttccataggcacagtgatcattgccagccagtggcacttctargtgaggaggctcttaggcgaggcccccaggatttgccctgtaggaaccgc

SEQ ID NO: 641 - DNA, 101 bp, Homo sapiens
acacattaaatcaccacttctagggaaaggttgagctcactcatagctctrttgatagtgacactgagagggtattaaatgttgaaaggtctaaaagggag

SEQ ID NO: 642 - DNA, 101 bp, Homo sapiens
gcatctggatgaatagatctacgatgaccatattgccttcactgtacatgrcctaaactcatctctctggaaagttaatctttcataacattaacatcagt

SEQ ID NO: 643 - DNA, 101 bp, Homo sapiens
ttctcttctgttgtttctacccgtgttcttctccgggatattatcagaaartaaacacaccaaaggaaataaacaaaatatgcatttccaatatattttcc

SEQ ID NO: 644 - DNA, 101 bp, Homo sapiens
atggaatttgcacattatatatgttatttatggaatacagatcattcattkaggcatttttctagattgtctttgagcttccctgaccaacttgcagttta

SEQ ID NO: 645 - DNA, 101 bp, Homo sapiens
ctgaacttaaacattatagacacacgctatgtctataatttttgacattayagacatgaaggtccttaatgggctagtgggcaaaagccatctaggaatca

SEQ ID NO: 646 - DNA, 101 bp, Homo sapiens
gttacctgatcggctgatccgggagttgaactgtaatcaggggcttgtagkagttagagctgtgtgggcctctgaggagctcccagcctcccaggagcggc

SEQ ID NO: 647 - DNA, 101 bp, Homo sapiens
tcactgccgttaagttgtagagttgctctaggtccctgcattcggctgtcrtatttcactgaacttactttgaagttgcttatgtcactctcaccattgcc

SEQ ID NO: 648 - DNA, 101 bp, Homo sapiens
gtatttttgttttttttttaagttttcagaactttaagatggtgtgtagayagatgcttttatgggccaagaaagcatgttgatatccattattttatttg

SEQ ID NO: 649 - DNA, 101 bp, Homo sapiens
ggtgctactgcttccagagacagcaaggtaaaagatgagacccttacagaygcaaatagttgacctgcatgtcaaattttacttattttttaagaaaataa

SEQ ID NO: 650 - DNA, 101 bp, Homo sapiens
attcatgtctaagcatttcgtagaaggatgcacgtgagaaaaagcacctgygctgtcatagcgatcctttggtgttttaagatgaaaaagttcaaagcatt

SEQ ID NO: 651 - DNA, 101 bp, Homo sapiens
aaacttccattaggaagtatgtgaaagaaactttcctttaaataaaaatgygtaagtgtttagaattgcccttgcaaagctctaaatcaatcacccagggc

SEQ ID NO: 652 - DNA, 101 bp, Homo sapiens
tagtcacctcctttgaacagctttctagtaacaggtccctggatccatggygcttatttttagaagagacagtagtatattattttgaggtcatggaatta

SEQ ID NO: 653 - DNA, 101 bp, Homo sapiens
tatgcttgttcccaatctccttgggagaaagcagtgtcaatcttttaccaycaagtataattttagctatagattttgtacaaataacttttatgagtcta

SEQ ID NO: 654 - DNA, 101 bp, Homo sapiens
gcatgtagatgcaagacatagcatttaagaatatcaatgtgtgtgcctacyatgccttactagctaaatattctactgttgtataacaggatgatttggtt

SEQ ID NO: 655 - DNA, 101 bp, Homo sapiens
tgtcccccaaccatctgtagacattcccaaaagcctccatcgcatatgctygtgcacccacttgtcagaagcatacccatgctgcaccgccccggatttgc

SEQ ID NO: 656 - DNA, 101 bp, Homo sapiens
aataccaaggagagcagagctgtgctgtcaagcccctgacaattcgtgaayttctgctgctgaaattattagtgctgccttggatcaagttccatttgtat

SEQ ID NO: 657 - DNA, 101 bp, Homo sapiens
tttttaatatcaattggaattgccgcaacacccaacactgacacacagttyccagagcaaagctccgtggtcagactcccaagctccttagtagtggtggc

SEQ ID NO: 658 - DNA, 101 bp, Homo sapiens
gttcaatacatctcaatgagaagcatgcaaccttaatccatgacgcttgtragtggagctatttttcaatctacgttaattttgaatttaactgtgtcaag

SEQ ID NO: 659 - DNA, 101 bp, Homo sapiens
acaaaattcttgaaggtcaatatgggatagcctcaagcctcggacacaaargagtttgtattcacactcaagcttttctttagggcccctaactgggtgct

SEQ ID NO: 660 - DNA, 101 bp, Homo sapiens
ctggattcaattctttctttgtttccatatccaatcctccatggatcattmtttttccttagcacttctgatgatgtttcccaggatacatccttagcctc

SEQ ID NO: 661 - DNA, 101 bp, Homo sapiens
taaacaagaatcacttttcccgtaatcttactacgaaaaatggtattaatygatatttgtacactaagatatggctaaaaagccaggtacctaagcccatg

SEQ ID NO: 662 - DNA, 101 bp, Homo sapiens
ttatgcttctttacaacttgtgcaactattacctaagataaagccctgaargaaaagaaactgtagtctgagtgactgtgagaaatcataaatgacagtcc

SEQ ID NO: 663 - DNA, 101 bp, Homo sapiens
cccacacttctcccatatctgtaacctctccatctcttttgttctgtctaytggcatataaacagattaaaatttctcccaccctaaaaattaagaataag

SEQ ID NO: 664 - DNA, 101 bp, Homo sapiens
gcatataaacagattaaaatttctcccaccctaaaaattaagaataagaaytctgtcaaatcaataaccaccctgactttctcctcttcacaacccaaaat

SEQ ID NO: 665 - DNA, 101 bp, Homo sapiens
aatacatcacatccattttatccatatcacttttcctgggtttggctaccrgcgcagattaatagttgtctttgcattatgcagtggaacttaatttctat

SEQ ID NO: 666 - DNA, 101 bp, Homo sapiens
atcagaacaagattctgaatgaaaacgtgttcccccaggtgagccatatgyagacgaatgcttgggatgctgggtagatgttgaaaaaaagttttgcccga

SEQ ID NO: 667 - DNA, 101 bp, Homo sapiens
gctcaggtttgcttcttaaacacagatttgaatacattactgtaaatctcygttttgcttttaggtcaaatagaaatggtcatggaatgacagcccagatg

SEQ ID NO: 668 - DNA, 101 bp, Homo sapiens
gaatcaatcacatccttgttgcctcccttttcttcaaccccatgttcaatyagtcgctgagctgctggtaaatccctaggagaaggagagtgatgtgtctc

SEQ ID NO: 669 - DNA, 101 bp, Homo sapiens
caaccctttcaaaaaatctctgggagttgaaccaggattgatcttgtggcraagaatcttcatcggctgctaggacagccattcagtctcactttcccatt

SEQ ID NO: 670 - DNA, 101 bp, Homo sapiens
ttctaccaagctcctaggtgatgatgttggggattcatggaccacgctttragaggcaaggataaagaaaactactgtatacgaattagggccacgatgtg

SEQ ID NO: 671 - DNA, 101 bp, Homo sapiens
aataaggaagcccatttattttatcattattacttttatcactaataacargctctttacacctacacatgagaatgacaatagcaaaggaaacaatcatt

SEQ ID NO: 672 - DNA, 101 bp, Homo sapiens
gaaaaagtattaatacttcctcagggtaacctccttcagcactatcagcarttacaatgagattgaatactaattaacctttaaatataggctttggggct

SEQ ID NO: 673 - DNA, 101 bp, Homo sapiens
tgtatcattctatggtaagactacgtttagctttgcaagaaactgtcaaaytgtcattcaacgtggctgtgtcatgttacattccctacaatgattgggag

SEQ ID NO: 674 - DNA, 101 bp, Homo sapiens
ccatcttgctgatttccaggttgcttcggggaccccaagagaattcatatkctggtggattggtgtgaggcacccgcctgtaactgagatatcgctgctgc

SEQ ID NO: 675 - DNA, 101 bp, Homo sapiens
accgcaaaatgtaccttgttgggtatttagcagaaggaaatgtgttgactrttacacatcccttatctacagtgcttgagactgttttgaatttcttattc

SEQ ID NO: 676 - DNA, 101 bp, Homo sapiens
gttgaatgatttcattttacatagattgccttttatgatttttatgatttyttcaactttcattttaggttcagggttacgtgtgtaggtttgttatatag

SEQ ID NO: 677 - DNA, 101 bp, Homo sapiens
cctaggcgaataaacaaaggaatgatttctccacttggatggacataccarttgtagcctgttggtctgtttctcaccctacttatcagagtaacctctcc

SEQ ID NO: 678 - DNA, 101 bp, Homo sapiens
ttggcttagattattttttaagtttcatattgtgccaccacgggcgggtcytctccatacagcagtgactgtaaaatcaaaccccactttcagtgagtgag

SEQ ID NO: 679 - DNA, 101 bp, Homo sapiens
cctgaaaatcagtttcttcccttcgattgacaaccaaggaggaagtcagtkggaagacctggggcattcataaagggacaagaatctttttctcattaagt

SEQ ID NO: 680 - DNA, 101 bp, Homo sapiens
acctttgtgatgctttatctcccaactgacactgaactacatactaaataygtattgctactatgttctcctaagcttttttatacatgctactttcttta

SEQ ID NO: 681 - DNA, 101 bp, Homo sapiens
actggccctgcagcactgagacactcaggagcccatgatcctccaccagcygtgaagcagcagagaaactcatggtccgaaaccgcaaccaaagcctccag

SEQ ID NO: 682 - DNA, 101 bp, Homo sapiens
aatacttttattaatatacaggaatccccccttacctgcagggcatccaaractcccgagtgaatgcctaaaaccacagatagtaccaagccctacacata

SEQ ID NO: 683 - DNA, 101 bp, Homo sapiens
agtgttggcagatgtcaaataactgcatttattcaaccagaactgatcatyatttagagtgaaatgatcaattattggagtaaaatgcattttgtttgcaa

SEQ ID NO: 684 - DNA, 101 bp, Homo sapiens
gcctgggttcaaatttggactctgccatttccttatctgtgacttggagarctcatttaaacttctcaattcttccattccctcatctataatggaaatgt

SEQ ID NO: 685 - DNA, 101 bp, Homo sapiens
tggtttctctctagttaaaaaggaatgttcaaaataactcaagaggttcgytttctggcaatttgcctctctagcaattcagaatttccttgtagtttttt

SEQ ID NO: 686 - DNA, 101 bp, Homo sapiens
gtttttccttaagaatggtgaagttgttttttttttttaaaaaaaggaaaygcatatgagttctggatagtttgaatacttggaaaaattattgtcctgga

SEQ ID NO: 687 - DNA, 101 bp, Homo sapiens
aaaccatcagaaaaaaaaaactatattcccctttccactctttatcataartataacttcaattaaaggaaataactttgatttatagttagaccacaaca

SEQ ID NO: 688 - DNA, 101 bp, Homo sapiens
cagttcacaacccatacccacagagaaacatacacatataccttatattayattggttcttttttttcctgaaacaaaaggtctcacatatttattactga

SEQ ID NO: 689 - DNA, 101 bp, Homo sapiens
ggaagtcaaaagttataagccaagtttcaaccgcttgcaaatgtacccctraaccccatgttgtacaagggtcaactgtactgttactgtcccctgttaca

SEQ ID NO: 690 - DNA, 101 bp, Homo sapiens
caaacctaggaggcaatattgcccagctgtaaggagcatgggctttagaayctctggttgctcttgttaatggtgcgactttaggcatgttatttaacctc

SEQ ID NO: 691 - DNA, 101 bp, Homo sapiens
ttggagttagtgtcagtagtgttgaatcattcaggactggatattaagtaygtaagggcaatagaagagcctggagcatatttcatatccctctatccctc

SEQ ID NO: 692 - DNA, 101 bp, Homo sapiens
cagcataatgcttggtatttgacatgttatcaagtatgaataggggagtakcaagggatatgaaaggggtcagaccaaaaagggattcattttatacctag

SEQ ID NO: 693 - DNA, 101 bp, Homo sapiens
ttacacccttcacagaattgcttgagggcacaagtacaaagaattaatatrttaattatcataagtgaatcattaaacagcaacagtaattaacagcttaa

SEQ ID NO: 694 - DNA, 101 bp, Homo sapiens
aagcactttaggtttttcagataacataatcagagaggcaagagtatattrtatttgcttttctgcctcttgtctgggcttaaaatatttcacttggagtg

SEQ ID NO: 695 - DNA, 101 bp, Homo sapiens
tctgatcgtctagttccaatatattctctgcctcttccttgatagcttaartcctgaattctgttcttaaatactgttgcagcttaagctgtcctgcctga

SEQ ID NO: 696 - DNA, 101 bp, Homo sapiens
gtggaaagtatagggactaagccaaaccaggagaaagtgtcaactccagtyaagatccagcagaaccctctggattggataagggacccagaataatccat

SEQ ID NO: 697 - DNA, 101 bp, Homo sapiens
ccaaagcagtttatctgtgtaccccaagactgcaaataaatttatagaacrgtgttgcctggtagaattttctataatgatagaaatgttttatgatctgt

SEQ ID NO: 698 - DNA, 101 bp, Homo sapiens
aaagcacagcttaacaagtactctgacacccagaaaaggcctacataaacycagtaggaaagaaacctaaaatagcagaagtgctggatgagagtaaggaa

SEQ ID NO: 699 - DNA, 101 bp, Homo sapiens
caatctcaacaaacattggaagaaaactgttcaaagccactggctcatagmctgctatctctatgaggatgtttaggatgatgtcattatgggttgaatcc

SEQ ID NO: 700 - DNA, 101 bp, Homo sapiens
tatttaatttggggctcagaagggctgaaaactgcattccatgaataagaraactggaaataatcaaagaactatatggactgcagcatctctctgccatc

SEQ ID NO: 701 - DNA, 101 bp, Homo sapiens
acagatgcaagtaaaaaaattaaaaagtattacggaaccacaatatttatragggacagtcctaagaatcccatgatttcccagattgataagggaacagt

SEQ ID NO: 702 - DNA, 101 bp, Homo sapiens
ggataagggagaatgtatatacaccaccaaaaaggagagagtcacaccgaraagtcagttttgagatcagtttagagaaaatgcaggccaaggcagtgtca

SEQ ID NO: 703 - DNA, 101 bp, Homo sapiens
cccttcccttcaagcaaaactcttgtgattcccctacactattttatggckccatgtgcttgtatattctgatccctctccccaaatgccctatcctgact

SEQ ID NO: 704 - DNA, 75 bp, Homo sapiens
accaataatttgattttgttgatayatccagatttgaccatttcaaggaagtaattcgtgtttatttaaattctc

SEQ ID NO: 705 - DNA, 101 bp, Homo sapiens
taagtatttctatatgctactattttttcttagattaaggtcctgaggatmtccaacttttgggttttagagaggtaacgtgttgcctttaacctctatta

SEQ ID NO: 706 - DNA, 101 bp, Homo sapiens
ccagccccaccttcctcttctttgaatcctgcccctcccttgctccagacytcaccaagtctctgcattacagttcacatcaaccctaagttgctctttcc

SEQ ID NO: 707 - DNA, 101 bp, Homo sapiens
gataggaacaaaaatggaatggtattcatctacatattatttgggcctctktactttttatgttgtaaatgaaggagataatttattcttaccacatactg

SEQ ID NO: 708 - DNA, 101 bp, Homo sapiens
agctacaacaggaaaaatgtgtggacatgaagggaacttgtgagtaggtgytgttgagtacatgcctgtgtgtgtatatgtgctagggacacctaccaggg

SEQ ID NO: 709 - DNA, 101 bp, Homo sapiens
tacattttactcttgtaccagtatcacaggttttgaatccaagaaatgtgrgtctatctacattgttctttttctaattattctgacgattttgtgtcctt

SEQ ID NO: 710 - DNA, 101 bp, Homo sapiens
ccaaggatgttcccatcaaatccttccctcatttgattttcacaacctgcraggaaggcaaggcaactggcatccatatggacatggaaaccgagggccag

SEQ ID NO: 711 - DNA, 101 bp, Homo sapiens
atctgattaattcagattagtttatggattagttcctctggggttggatarcttctcttggctcaatcagccatgtcaggggaatgacattgctaatgaag

SEQ ID NO: 712 - DNA, 101 bp, Homo sapiens
aagtagggtctgtatggcaaggacattacctatcttgtttaccatgaaatygccagtgcctagtggatcaccacctagtacacgctcaataaacactaggt

SEQ ID NO: 713 - DNA, 101 bp, Homo sapiens
acacgaaactgttacccatgccttttcattttccccttcattatcctctgyaccttacatttctaaatggaaacccttcaatgactacctacttaactctc

SEQ ID NO: 714 - DNA, 101 bp, Homo sapiens
gatgatgtgcttacatttttctgcaaccgatcttctgacattttctcgttyccccagccacgagattgtaatttaacctcaactttttgtgtgtgtgcaag

SEQ ID NO: 715 - DNA, 101 bp, Homo sapiens
cctggctgagctctgcccgcctggaggctcccacaggatggccctggggaytgctgctgcactcggtaggtgcccttggccagggtcttcctgatgggctc

SEQ ID NO: 716 - DNA, 101 bp, Homo sapiens
tggcacacacaggaagcttgcatctgacaacaggaaggctggaacgccacktggatttgctcaaggagggtacaagcatctcctgctcattgtctcctttg

SEQ ID NO: 717 - DNA, 101 bp, Homo sapiens
aaaggattttccccacatttatagctctgaagttgagctttttatcacctygctttttggctcccaagtcttgctgctgggtagaattacctggaaagctg

SEQ ID NO: 718 - DNA, 101 bp, Homo sapiens
tgagtatttagattctcaagatgactatttcaaaggacagtagttccttgyatgcactaaaaataccccgaaacatgaatacttcttttttaaaatgaatc

SEQ ID NO: 719 - DNA, 101 bp, Homo sapiens
tgagtgtctttgacagtaactccttcatagatgctttcttatgatgtaccmtttaattttgatgaaggtcctgtgaaataagcagagcagattttatgatc

SEQ ID NO: 720 - DNA, 101 bp, Homo sapiens
gttttggaaatgttgttgcattgtcactttctgcagtagaaactgaaaaaygagaaacacactgtgtttgactggaagcccaaaggagacaaaatgttttc

SEQ ID NO: 721 - DNA, 101 bp, Homo sapiens
caaaatagcatataatctagtttggttgaccctttgctttccacaggcacrgaatgggaaataaggatggaaatgagaattggggatgtattgcagaggaa

SEQ ID NO: 722 - DNA, 101 bp, Homo sapiens
cagagcaggaaagtgagctcctcagcagagaccaggctgggatgaggacamcgcggtgcagaagaaaatctgcctggccgtggtgcctaaagctgccatgc

SEQ ID NO: 723 - DNA, 101 bp, Homo sapiens
cacgatataggaagaccaaccaattcttgaaaagcttttttcttttcccarttgcttcagtgatagccacacatttcaataaacccaattttcctccatct

SEQ ID NO: 724 - DNA, 101 bp, Homo sapiens
tctgggccataagatataccttaacagatttaaacaagtagaaatgatacraagtgtgctctaataatgccataatggagctaaatgagaaatgtaaaaaa

SEQ ID NO: 725 - DNA, 101 bp, Homo sapiens
cctggtccctggaggaacagtagcctctgtctgagtcctaaactggggcarcaggccgggcacaatgtctcaagcttgtaatcctagcactttgaggcacc

SEQ ID NO: 726 - DNA, 101 bp, Homo sapiens
gaaataggatttcctcaataaggacaaaatggctcagggccaaaatgaaarcatcactcagcactttttttttttttttacttttatagtcaatgcaaaga

SEQ ID NO: 727 - DNA, 101 bp, Homo sapiens
gtctggtgtccgagcagcgtgtggtcctgggaacatcttacatgaagtgargtgtccatccttgggtgggtccctctgactcaaggcgagtcttgtggagg

SEQ ID NO: 728 - DNA, 101 bp, Homo sapiens
gtaaaaaaaactgaaggtagtaaatgtggtcgttcagagaaattcagagtraaatgaaggagaatgagggacaggatggcaatactaatagataagggagc

SEQ ID NO: 729 - DNA, 101 bp, Homo sapiens
tacttctaggtatacttctaggtaaaactccccaagaaacactcatatatrtgcacaaggaaacaaacataagtatgttccatgaagtactatttgcgaca

SEQ ID NO: 730 - DNA, 101 bp, Homo sapiens
actgaagactccaagctatatggactgaatccacccccaattcccccgccyaattcatacactgaagccctagacccagtgtgactgtactggagacagag

SEQ ID NO: 731 - DNA, 101 bp, Homo sapiens
tacttacccccttcagataaacagaaaatgcaactctatgtaaatattccytaagaatattttgcagcacactggaattaaattagtgctaaagatgatga

SEQ ID NO: 732 - DNA, 101 bp, Homo sapiens
gttccacttacacaaacgtccacaacacataaatctagaaacagaaactaygttagtggctgcctagggtttaggatgaggagggtagatgtgaagaatga

SEQ ID NO: 733 - DNA, 101 bp, Homo sapiens
atgtggtgatgattaaccttgtcaacttattttttaaataatcctcatcgyttataccattgtagtaaagggttcccctctcccatgcagcaagtccagaa

SEQ ID NO: 734 - DNA, 101 bp, Homo sapiens
gaggaaccacccctctccctctctctgccaatctgtattggggcaaggttkggaagtactggcgagggtattacatttcaagaaacatgaccagggaagcc

SEQ ID NO: 735 - DNA, 101 bp, Homo sapiens
aagtcaaaagactagatagagaaatgatgtccagggagctcataatctgcytgtgcaagaattctagtttctagaaagtcactgattaataaattcatgtg

SEQ ID NO: 736 - DNA, 101 bp, Homo sapiens
ctacacaaagccctcttcaacagatagcataaacgctaccctgtaaaatcrccagcaagcctttgtctccttgcagtcagtttctctctgctgcctgccta

SEQ ID NO: 737 - DNA, 101 bp, Homo sapiens
tattgttttctctttaatggtgaaacttgatagggaacctaaaaagaattktaagactgcattcacttaatttgaagcttaactagaaatttgtttgctgt

SEQ ID NO: 738 - DNA, 101 bp, Homo sapiens
ccactctactgcttgggagtaagcggccaccaaaaccccgcttccagcagrtgctaggagcaacatgacaggaaaaacacaacctaattaaaatggtagag

SEQ ID NO: 739 - DNA, 101 bp, Homo sapiens
ctccatcctcatctgtctggtcgctgtctccacttctctcttcagatatcrgttcaggcccagctgcaatagatacctgcatgactccacccaaggacaaa

SEQ ID NO: 740 - DNA, 101 bp, Homo sapiens
gatgacttactttgctgccaaagggctgggcctgggcctgggcctctgagycaggttctccatcctcatctgtctggtcgctgtctccacttctctcttca

SEQ ID NO: 741 - DNA, 101 bp, Homo sapiens
acttctaaattaccaccatccaggttgcatctatttatggttccattcccygaactgatccaataaagcttgttttccacatagtctatcgatagacctgt

SEQ ID NO: 742 - DNA, 101 bp, Homo sapiens
tcacagtaacccccagtcctcaaaacatcaacaataaacacagacctgcaytgattgtggtattctgggtatttctataacatttctaggtttctgtagat

SEQ ID NO: 743 - DNA, 101 bp, Homo sapiens
atcttggtttttctgccttgacctttggctctttctaatgtaattggctcmgactccatttctggccatctgaactctggttccaagaattaatccaggtg

SEQ ID NO: 744 - DNA, 101 bp, Homo sapiens
atctctccttaattattacagaaaaaaatgttattaaagaaacaatcaggkgatccagcaaaagctgacaatgcacagtagtttagaaaccataagatgca

SEQ ID NO: 745 - DNA, 101 bp, Homo sapiens
gaggttattagcatccccttttacagaagaaaaaactgagaaaccaagcayatacagctggtaagtaacgtagtctgggtgcaaaaccacgaagctcatga

SEQ ID NO: 746 - DNA, 101 bp, Homo sapiens
cttctgctttcaaaaggaattgaagaaaccctaagataaaagagacaagayacactcaagccattcaaagaacaaggacggcacagaaagtacaggttata

SEQ ID NO: 747 - DNA, 101 bp, Homo sapiens
tctgggaacagactacttgctgaaacgaacaaattcccaggcagttgaaarcctttgtgtttcctactgggaataacctgcattcacaaattcattagcct

SEQ ID NO: 748 - DNA, 101 bp, Homo sapiens
tctgcccactgtttctctccttctgctgcagatctttgagctgaataagcrtatttcagctgtggaatgcctgctgacctacctggagaacacagttgtgc

SEQ ID NO: 749 - DNA, 101 bp, Homo sapiens
tctcagttttggagaccaaaagttggctgttttggtgggctgaaatagagytgtgggaagggccccactccagatggaggctctgggggagaatccttttt

SEQ ID NO: 750 - DNA, 101 bp, Homo sapiens
tatatatgtcaagcaataccttagtaaggtactcacttattttatccctartggcatattaatcaggcaatgtcatagatctctggttactattccacctc

SEQ ID NO: 751 - DNA, 101 bp, Homo sapiens
cactagttattggcggtggtgaattcagtttacatggctctgaattcatarcaagtttatttctttaggaaaatgcaaatagttattgtggttggcagaat

SEQ ID NO: 752 - DNA, 101 bp, Homo sapiens
tctgaagggctaagcaagggtaagttgtttatgctgttgcaggaaccacartgatgggaaagaaaaatgatatggtatttccatcccgggccttaaaataa

SEQ ID NO: 753 - DNA, 101 bp, Homo sapiens
aaatgttgactatatacctgcttgataataagaaacattcacctctcttcrtttaagttcaacttaaagaagaaacatttttgaaaagtgagaagtgtgtt

SEQ ID NO: 754 - DNA, 101 bp, Homo sapiens
caagatagccttctttagaatatgatttggctagaaagattcttaaatatrtggaatatgattattcttagctggaatattttctctacttcctgtctgca

SEQ ID NO: 755 - DNA, 101 bp, Homo sapiens
gctttataactgagatgtgtacttcaggcttgcatgggaattgtctgtacrgcccacaaactggcccccaggtctttgggactccttcctgtaacttagtg

SEQ ID NO: 756 - DNA, 101 bp, Homo sapiens
ttattatctctgaatcacagatgagtaaactgaggcacagaggttttttgktttttttttcccttaaggacagaaaacagcatattcaaaccgaggcatgt

SEQ ID NO: 757 - DNA, 102 bp, Homo sapiens
aatcacaggtttttatcaataaatgtccagctgggtacattcctccctctmtctaaacacaactcctgccggtcaggcactgtgtcctagaacctttgccat

SEQ ID NO: 758 - DNA, 101 bp, Homo sapiens
cactttgctgctgctctttctgcctctgtgaccactccttataggttcctyttcttcttgtgcctgcccctttaatgctgatattgatgttttctcccaag

SEQ ID NO: 759 - DNA, 101 bp, Homo sapiens
cacaaaagaaatgtttcctctcacagttggtgaagctagatgtctaaaaaycaaggtatcagtagggccatgctcccactgaaggctgtagggaagattcc

SEQ ID NO: 760 - DNA, 101 bp, Homo sapiens
ccttgtacttctccttggtgtcatgaagacaaatagcattaaaaaaagttytcccagtgaagcagctctcattttctcctctctcatccccttccaaacat

SEQ ID NO: 761 - DNA, 79 bp, Homo sapiens
gagcgtagctttctagagtgtgcgagtggtggctagatgtgctttgtttcrtgtgctgtgcatttcagtgctagtgtga

SEQ ID NO: 762 - DNA, 101 bp, Homo sapiens
ttccctcattgccaatcaccccatttagttatgaaaatacttcattggtartagtggccaaacaggcaaatatctattcagtaattagatgaataaatggg

SEQ ID NO: 763 - DNA, 101 bp, Homo sapiens
aaaaacaacaaaaatacaaaattttcatgatgatataataggaagctctcraaggttggattcaggtaaggaaatgggggaaagtttcctgataccctgac

SEQ ID NO: 764 - DNA, 101 bp, Homo sapiens
cagcaggagtggactgaatagcgtgcccctgggaggtttgtcttcctaagyagatccaatcggtcttcttgttctgatgaagtaaaacagagtggatatcc

SEQ ID NO: 765 - DNA, 101 bp, Homo sapiens
aaacaaactgttctaaattcaaggagtctctgccagttatgtgactttgcrtgactgactctgctttacccctccaggcccaagagacaaggctgtccaga

SEQ ID NO: 766 - DNA, 101 bp, Homo sapiens
taatctcccagaggtgtttccttttgttactctccaaaatgaaaagtctayttttttcttatcaaagccatacatgcttcctgtaaaatcaactcagataa

SEQ ID NO: 767 - DNA, 101 bp, Homo sapiens
tcacagggaatggggtttcttttatcactgacgatagcaagacctactttyttgctctggacagctcctatgaaaatatggcattcagaactgcttccctg

SEQ ID NO: 768 - DNA, 101 bp, Homo sapiens
gaaaggatgataaatcttaggaataataccaatggcattaatgtaatcccrcgtaagtttcgaaaaacctttccaagtataaattcagtaagaaaagctgg

SEQ ID NO: 769 - DNA, 101 bp, Homo sapiens
tgccgttcttggcatcatttctatttggctgtgagtcgtccgcttgatgcrtggtccacagctgattttcatgccccaaacaatccccatcgaaggtcaca

SEQ ID NO: 770 - DNA, 101 bp, Homo sapiens
caatggttaagaattaatttctatgtgttttgttatccgttaaacacaggytgtgagctagcaagaaacaagatacttttggaggcttagtgacttttttt

SEQ ID NO: 771 - DNA, 101 bp, Homo sapiens
aacagaggacattctgttttggagccatgttcccctgtccctggaataccycgctacttattagaaaagcagaaatgcaaaaaatcacagacatgtggggg

SEQ ID NO: 772 - DNA, 101 bp, Homo sapiens
tctttctgggctaacaccaagggggtggcagggctgtctgtgttcctgctrgtggttataagggagaaattccttccttgctttttccagatcctagaggc

SEQ ID NO: 773 - DNA, 101 bp, Homo sapiens
gaaaagcttcctagagaaggggcagctggaacctgaagaacaaaaccagarctgacgacgacggatgaggcaggtgtttcaggtggcagagcaacacaggc

SEQ ID NO: 774 - DNA, 101 bp, Homo sapiens
caatttctccatttttaaaattggtaagtcccccagcccaaggatatggtragtgattgtgtgacctccagaaaccacacttctcccatggatctttgcag

SEQ ID NO: 775 - DNA, 101 bp, Homo sapiens
cttcctcttttcctttgttctctattgcctttacctattttaaaaagtttyaaattattagccagtcgggttttagtttaaattgtaaggtctagctccag

SEQ ID NO: 776 - DNA, 101 bp, Homo sapiens
ataggtgagagggatctagattacgaaaggcctctgaagccagggagaaaytgaacttaatatgacaggtagtgaggagtcagtgtgagttcctcctgggc

SEQ ID NO: 777 - DNA, 101 bp, Homo sapiens
cttttccatttccatttttacttcctctcctacagtctcttttaaatccayaaccaattaggttttcattccaccaaagctgctcattaaaatcccttact

SEQ ID NO: 778 - DNA, 101 bp, Homo sapiens
aaggcatttaggtcctgggcatgcaggtctgtctcctctcactagaatgcmagttctggatggtcagcaattttgtttcattcactgtcatgggctgtgac

SEQ ID NO: 779 - DNA, 101 bp, Homo sapiens
tctctctcgctgctatcaggttgtcagtgtttgtccttgctgagccaggtragcaggcttctgatgtatttacgtaggtcaatggtctctaaaattatttg

SEQ ID NO: 780 - DNA, 101 bp, Homo sapiens
ggcatcacattagagactccaaaatcagactacctacttcaaatattaackctgtggccttaagatattaaacccttatgtgtctcagtttcttcatctat

SEQ ID NO: 781 - DNA, 101 bp, Homo sapiens
aagacaagcaaatttttcatcaatgaagttatacaaatgtgaaacatacaraaagatgttcaacactattcattattggagaaatgcaaattaaaaccaca

SEQ ID NO: 782 - DNA, 101 bp, Homo sapiens
tatgtgtgagtgtacatatatgttttaaaaatccctagcaagagtaagtaygttatttggtcagtcagctgttaaaacttccactttctccagttgtctgg

SEQ ID NO: 783 - DNA, 101 bp, Homo sapiens
taactggcaacacatgcactttcttttgagcttttaaaaacattgctccaytgctatcattgtagacccccaaggagaaggtaccccagcctcctggaaac

SEQ ID NO: 784 - DNA, 101 bp, Homo sapiens
ggtccaaaagggccacagtttgctggcagaaaccatacgaagtagattttrttgttacccccattttaaagatgaagaaactgagtcccagagaggttcag

SEQ ID NO: 785 - DNA, 101 bp, Homo sapiens
tctaacctttggtgtgcgctgtccctaagggaggaaggagtgcagctcacmaaagcccccttgaaacaaaggaaatgtgaacgcaacaccaaccactgaag

SEQ ID NO: 786 - DNA, 101 bp, Homo sapiens
gcctttatctctgcttcttctacccaacaggtgactccttttagctagggyatcacttatacctaacaggggactcaatttagccaggatttcactctggc

SEQ ID NO: 787 - DNA, 101 bp, Homo sapiens
atctttccactggagggaaattgggttcatagagtagaaatactttgcccragcctcaacagctgctaagaggtgcaatgaaaactcaacttgaggctgtc

SEQ ID NO: 788 - DNA, 101 bp, Homo sapiens
ccatcttggcatcattaaaaagggccaaccaagatgttacatgtccacgaygtgacacaggaggaatcaaacagcctgcctatgaagtagtcttgacaaca

SEQ ID NO: 789 - DNA, 101 bp, Homo sapiens
tttagctatctgccatttccagacacttcatgctctctgagtcttatcttycactcccagaagattgtcaaagtattttccaaaacaaagatagtttcccc

SEQ ID NO: 790 - DNA, 101 bp, Homo sapiens
aacataactttggggaatagctatagatactaaaggggcaacataaaacakttattgattacaaagtgtatgaagacccagttgcttggcagagtgatatc

SEQ ID NO: 791 - DNA, 101 bp, Homo sapiens
aatggggcaggaggtagaatggcacaggaattcaagtagaggaggatttaycatgaagctaatgaagtctaagtttcagggcttctcacctgtgcaggcca

SEQ ID NO: 792 - DNA, 101 bp, Homo sapiens
tatagcatctatttataagccacacacaccatcttatattaatgcttataytgtcttggctcacttagatacaaataaaggtttgcatctgatagaggaat

SEQ ID NO: 793 - DNA, 101 bp, Homo sapiens
ttaatggatgaagatatgtagacatctatggtgttctgggaagctgagcaygtctgatataaggcatgtgaggtttaaatgcatgcatgtgttagatatgt

SEQ ID NO: 794 - DNA, 101 bp, Homo sapiens
gccaagttcccaaggtcgcagcaaggtaaatgggattccacttgtgttcgraaaatctgtttataggccttctcctgaatcaaaacacacaggggaaaagc

SEQ ID NO: 795 - DNA, 101 bp, Homo sapiens
ataagggtgaggctagatctgctatgtccgaaatggcagccactggatgcrtgactagatttacattaattacaatgattctaataaaaaatgaagttctc

SEQ ID NO: 796 - DNA, 101 bp, Homo sapiens
aaccatgccttgttttgcgtcttctcaaagaaccccgggggcacgtggccracaatgtacacctacaagggaggggttcccagaagaggctcacagatgcc

SEQ ID NO: 797 - DNA, 101 bp, Homo sapiens
aaccccctcctttctcctgtactgatgactctgtagctttaaccagggcgrcggtgtcactctaaatgtcaccttggcattcagccccatagagtggggaa

SEQ ID NO: 798 - DNA, 101 bp, Homo sapiens
caagcaaaagaaccttgaataagccaatatttcactcataatgtgagtgcraaacatgaaacccaactttccgggttcaaatgccaagtacagctagagtc

SEQ ID NO: 799 - DNA, 101 bp, Homo sapiens
gccctggggctaggataagcttcttctctgattcaaagaagcattctccaragttgcttgccagataccaggttctgagctagttggcctcccaaaaaccc

SEQ ID NO: 800 - DNA, 101 bp, Homo sapiens
aaatatttatgatgttgtctaaaaatgagtaggtaacacactccacattaycagtacagagactattctagcatcaatgaatgccaccatagataactatt

SEQ ID NO: 801 - DNA, 101 bp, Homo sapiens
atggcagtaagtcatacccaaatttggttcacttcactcaaatatttgtgrggcacttaacgattaaagggtttgtaggtactttgtttaatgaataaatt

SEQ ID NO: 802 - DNA, 101 bp, Homo sapiens
gggactttctggacactaccacatggagactgaagatgaagctaacacttyccagagcaagctgagtgacagacagaaatcaaagcctgatgataccattt

SEQ ID NO: 803 - DNA, 101 bp, Homo sapiens
taataatggaggaaacccggtgggtgtgaggtatatgggagttttctggaytctttgcagtttttcagcaatctaaaactgttccaaaataaagtttagac

SEQ ID NO: 804 - DNA, 101 bp, Homo sapiens
ttaggtcttcctaagaatgtatttctgcctcagaatgcacaatgttttcayataaatgtcagtatgattagggtttattagcaattgtaaaaaattcaaca

SEQ ID NO: 805 - DNA, 101 bp, Homo sapiens
tctactccaacttgttggaaagtagtagtagaatacaaactagtcaaatamaccacgttgtgtaatgaactgaaactttaacttattttgttggagtcaaa

SEQ ID NO: 806 - DNA, 101 bp, Homo sapiens
tgccctggagacgttttccccattgtcttggtaactaacattcagctccgygtgcagcaccaacttacttatgcaaatttctgtcactggtttgaatttct

SEQ ID NO: 807 - DNA, 101 bp, Homo sapiens
tcaggtctactcatctgtaaaatgagaataataactgccaccaactccctrgattatgttgaggatttgattaggtagtgtttatggagcatggcacgtgt

SEQ ID NO: 808 - DNA, 101 bp, Homo sapiens
actccacctctctctgatgagaagaggtaagtaggatttacagataagcayacgagaagcaggggaagatgctaaggcaaagaaggggcctgaacaccacc

SEQ ID NO: 809 - DNA, 101 bp, Homo sapiens
tgtgaaatttcaagttttattcttccattgttccatttgtgacctaatctrtgaagcctttcttatcttttccaagtaaaggatcactctcttcttacttc

SEQ ID NO: 810 - DNA, 101 bp, Homo sapiens
acagaggctgtccttaaaggagctgagcctcccttctctcaagggcatctrtgtctgcgaatccatccaggctgatgactgtcaacctggggctttttgtt

SEQ ID NO: 811 - DNA, 101 bp, Homo sapiens
tcttctctgaaagctgaattaactagtcaggaatcgcagatctccacttaygaagaagaattggcaaaagctagagaagagctgagccgtctacagcaaga

SEQ ID NO: 812 - DNA, 101 bp, Homo sapiens
tggcaggagtgatgctggcctaatgacaacctcagccaatgaccaacctcrtggagatactttagagctaaaaccgtatcttaatgttgtcatgcattggc

SEQ ID NO: 813 - DNA, 101 bp, Homo sapiens
ggcagaccataatgattcaaccacttggattctacaaacaatactttaacrtggaaatgtgtacttggatgaagaagagagaaggcaatgcctgatttttc

SEQ ID NO: 814 - DNA, 101 bp, Homo sapiens
agcactgaagaggtctttgctgactctggctcaggaattagaagtttctcrgcaagggccatttaaattgggccttgatggatgtataggagctcaataag

SEQ ID NO: 815 - DNA, 101 bp, Homo sapiens
agttacaagagctcactgaccaacacaaactcaggttagcagagcttttcygaaattccacccttttcctctgcgttcagtgctacttactcccttcactt

SEQ ID NO: 816 - DNA, 101 bp, Homo sapiens
gctccgctattcagtttcaggtagggacatagagtcttagagagggtgagrcatttatacaaggctacatagcaagtagaaggcaaaaccaggacaggatc

SEQ ID NO: 817 - DNA, 101 bp, Homo sapiens
ctttactgagctcctattagatgctttgcatgaggtatttcaatttaatgygtaggaactgtggcttatttgacttgttacacccaacagcgcctggcacc

SEQ ID NO: 818 - DNA, 101 bp, Homo sapiens
cagtgctcccagtggtgggtacctaccccagagaactctcacatgtatcaraagtgggcatgtatacagttcagaactgtccatcatggtcagagttgaag

SEQ ID NO: 819 - DNA, 101 bp, Homo sapiens
tgcaaactgggaagctgagggtgcccatgttttctgttatgtggactaggragcagagaatgccacccacaaggaaagagaaaactcaagcatatgttcag

SEQ ID NO: 820 - DNA, 101 bp, Homo sapiens
caccagcctcattggccttgtccctgaatcttacacacctaaatgcaaacrcaccttccaattatctgcttgttcttctttttatccacttctttgtctcc

SEQ ID NO: 821 - DNA, 101 bp, Homo sapiens
cttacatttcatttctttcattcttattccttaatagatatgtttgttctytctgcctgttctttttcttattcctccatctttgcttgtctatcccaccc

SEQ ID NO: 822 - DNA, 101 bp, Homo sapiens
tcagagaccccagggaattcacatgtgtattgctttgtaaatgttctgacratgctctcaggtgccagggctgtgtcttcagcatcagtttgggagttcct